(19) United States
(12) Patent Application Publication
(10) Pub. No.: US 2025/0017108 A1
(43) Pub. Date: Jan. 9, 2025

(54) LIGHT-EMITTING DEVICE AND ELECTRONIC APPARATUS INCLUDING THE SAME

(71) Applicant: Samsung Display Co., Ltd., Yongin-si (KR)

(72) Inventors: Hyejeong Park, Yongin-si (KR); Beomjin Kim, Yongin-si (KR); Ha Hwang, Yongin-si (KR); Jiwon Kwak, Yongin-si (KR)

(21) Appl. No.: 18/735,109

(22) Filed: Jun. 5, 2024

(30) Foreign Application Priority Data
Jun. 14, 2023 (KR) 10-2023-0076426

(51) Int. Cl. H10K 85/60; C07C 211/54; C07D 307/91; C07D 333/50; C07D 333/76; C09K 11/06; H10K 50/12; H10K 50/18

(52) U.S. Cl. CPC H10K 85/6574; C07C 211/54; C07D 307/91; C07D 333/50; C07D 333/76; C09K 11/06; H10K 85/615; H10K 85/633; H10K 85/636; C09K 2211/1007; C09K 2211/1011; C09K 2211/1018; H10K 50/12; H10K 50/181; H10K 85/626; H10K 85/6576

(57) ABSTRACT

A light-emitting device including a first electrode, a second electrode facing the first electrode, and an interlayer arranged between the first electrode and the second electrode and including an emission layer is provided. The interlayer includes a first compound, a second compound, and a third compound. The first compound is represented by Formula 1 and includes deuterium, the second compound is represented by Formula 1 and includes deuterium, the first compound and the second compound are different from each other, and the third compound is represented by Formula 2.

### CROSS-REFERENCE TO RELATED APPLICATION

This application claims priority to and the benefit of Korean Patent Application No. 10-2023-0076426, filed on Jun. 14, 2023, in the Korean Intellectual Property Office, the content of which is incorporated by reference herein in its entirety.

### BACKGROUND

### 1. Field

One or more aspects of embodiments of the present disclosure relate to a light-emitting device and an electronic apparatus including the same.

### 2. Description of Related Art

A light-emitting device may include a first electrode, a hole transport region, an emission layer, an electron transport region, and a second electrode, which are arranged in the stated order. Holes injected from the first electrode may move toward the emission layer through the hole transport region. Electrons injected from the second electrode may move toward the emission layer through the electron transport region. Carriers, such as the holes and electrons, recombine in the emission layer to produce excitons. As the excitons transition (i.e., relax) from an excited state to a ground state, light may be generated (e.g., to display an image).

### SUMMARY

One or more aspects of embodiments of the present disclosure are directed toward a light-emitting device having a low driving voltage, a high luminescence efficiency, and/or long lifespan, and an electronic apparatus including the same.

Additional aspects will be set forth in part in the description which follows and, in part, will be apparent from the description, or may be learned by practice of the presented embodiments of the disclosure.

According to one or more embodiments, a light-emitting device includes
a first electrode,
a second electrode facing the first electrode, and
an interlayer arranged between the first electrode and the second electrode and including an emission layer,
wherein the interlayer includes a first compound, a second compound, and a third compound,
the first compound is represented by Formula 1 and includes deuterium,
the second compound is represented by Formula 1 and includes deuterium,
the first compound and the second compound are different from each other, and
the third compound is represented by Formula 2.

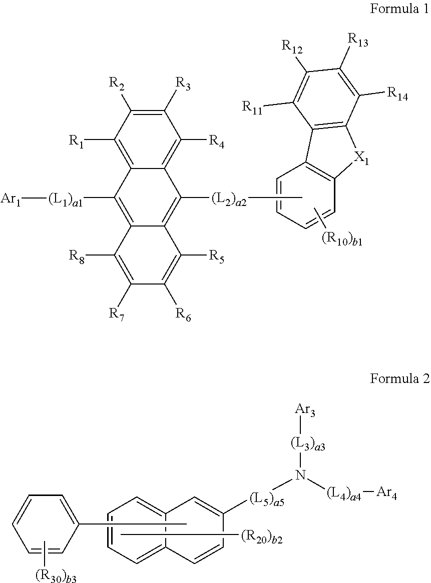

In Formulae 1 and 2,
L₁ to L₅ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
a1 to a5 may each independently be an integer selected from 0 to 2,
Ar₁, Ar₃, and Ar₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₁ may be C(R₁₅)(R₁₆), N(R₁₅), O, or S,
R₁ to R₈, R₁₀ to R₁₆, R₂₀, and R₃₀ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b1 may be an integer selected from 0 to 3,
b2 may be an integer selected from 0 to 6,
b3 may be an integer selected from 0 to 5,
R₁₀ₐ may be
deuterium, —F, —Cl, —Br, —I, a hydroxyl group, a cyano group, or a nitro group,
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof,
a C₃-C₆₀ carbocyclic group, a C₁-C₆₀ heterocyclic group, a C₆-C₆₀ aryloxy group, or a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof, or
Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be
hydrogen, deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, or
a C₃-C₆₀ carbocyclic group or a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

According to one or more embodiments, an electronic apparatus includes the light-emitting device and a thin-film transistor electrically connected to the light-emitting device.

According to one or more embodiments, electronic equipment includes the light-emitting device and may be at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, and combinations thereof.

### BRIEF DESCRIPTION OF THE DRAWINGS

The accompanying drawings are included to provide a further understanding of the above and other aspects, features, and advantages of certain embodiments of the present disclosure are incorporated in and constitute a part of this specification. The drawings illustrate example embodiments and, together with the following description taken in conjunction with the accompanying drawings, serve to make the principles of the present disclosure more apparent. In the drawings:

FIG. 1 is a schematic view of a light-emitting device according to one or more embodiments of the present disclosure;

FIG. 2 is a schematic view of an electronic apparatus according to one or more embodiments of the present disclosure;

FIG. 3 is a schematic view of an electronic apparatus according to another embodiment of the present disclosure;

FIG. 4 is a schematic perspective view of electronic equipment including a light-emitting device according to one or more embodiments of the present disclosure;

FIG. 5 is a schematic perspective view of the exterior of a vehicle as electronic equipment including a light-emitting device according to one or more embodiments of the present disclosure; and

FIGS. 6A-6C are each a schematic view of the interior of the vehicle that includes electronic equipment including a light-emitting device according to one or more embodiments of the present disclosure.

### DETAILED DESCRIPTION

Reference will now be made in more detail to embodiments, examples of which are illustrated in the accompanying drawings, wherein like reference numerals refer to like elements throughout, and duplicative descriptions thereof may not be provided the specification. In this regard, the present embodiments may have different forms and should not be construed as being limited to the descriptions set forth herein. Accordingly, the embodiments are merely described, by referring to the drawings, to explain aspects of the present description.

As utilized herein, the term “and/or” includes any and all combinations of one or more of the associated listed items. Expressions such as “at least one of,” “one of,” and “selected from,” when preceding a list of elements, modify the entire list of elements and do not modify the individual elements of the list. For example, throughout the disclosure, the expression “at least one of a, b or c” indicates only a, only b, only c, both (e.g., simultaneously) a and b, both (e.g., simultaneously) a and c, both (e.g., simultaneously) b and c, all of a, b, and c, or variations thereof.

Unless otherwise defined, all chemical names, technical and scientific terms, and terms defined in common dictionaries should be interpreted as having meanings consistent with the context of the related art, and should not be interpreted in an ideal or overly formal sense. It will be understood that, although the terms first, second, etc. may be used herein to describe various elements, these elements should not be limited by these terms. These terms are only used to distinguish one element from another element. Thus, a first element could be termed a second element without departing from the teachings of the present disclosure. Similarly, a second element could be termed a first element.

As used herein, singular forms such as “a,” “an,” and “the” are intended to include the plural forms as well, unless the context clearly indicates otherwise.

It will be further understood that the terms “comprise,” “comprises,” “comprising,” “has,” “have,” “having,” “include,” “includes,” and/or “including,” when used in this specification, specify the presence of stated features, steps, operations, elements, and/or components, but do not preclude the presence or addition of one or more other features, steps, operations, elements, components, and/or groups thereof.

As used herein, the terms “use,” “using,” and “used” may be considered synonymous with the terms “utilize,” “utilizing,” and “utilized,” respectively.

As used herein, the term “and/or” includes any, and all, combination(s) of one or more of the associated listed items.

The term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

It will be understood that when an element is referred to as being “on,” “connected to,” or “coupled to” another element, it may be directly on, connected to, or coupled to the other element or one or more intervening elements may also be present. When an element is referred to as being “directly on,” “directly connected to,” or “directly coupled to” another element, there are no intervening elements present.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the drawings. It will be understood that the spatially relative terms are intended to encompass different orientations of the device in use or operation in addition to the orientation depicted in the drawings. For example, if the device in the drawings is turned over, elements described as “below” or “beneath” other elements or features would then be oriented “above” or “over” the other elements or features. Thus, the term “below” may encompass both an orientation of above and below. The device may be otherwise oriented (rotated 90 degrees or at other orientations), and the spatially relative descriptors used herein should be interpreted accordingly.

In this context, “consisting essentially of” means that any additional components will not materially affect the chemical, physical, optical or electrical properties of the semiconductor film.

Further, in this specification, the phrase “on a plane,” or “plan view,” means viewing a target portion from the top, and the phrase “on a cross-section” means viewing a cross-section formed by vertically cutting a target portion from the side.

In present disclosure, “not including a or any ‘component’” “excluding a or any ‘component’”, “‘component’-free”, and/or the like refers to that the “component” not being added, selected or utilized as a component in the composition, but the “component” of less than a suitable amount may still be included due to other impurities and/or external factors.

### Light-Emitting Device

According to one or more embodiments, a light-emitting device includes
a first electrode,
a second electrode facing the first electrode,
an interlayer arranged between the first electrode and the second electrode and including an emission layer, and
wherein the interlayer includes a first compound, a second compound, and a third compound,
the first compound is represented by Formula 1 and includes deuterium,
the second compound is represented by Formula 1 and includes deuterium,
the first compound and the second compound are different from each other, and
the third compound is represented by Formula 2:

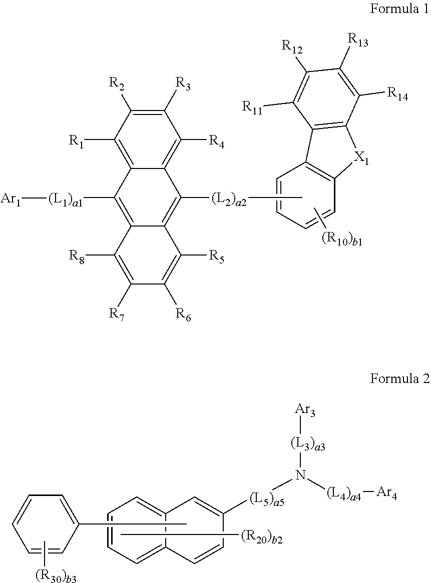

wherein, in Formulae 1 and 2,
L₁ to L₅ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
a1 to a5 may each independently be an integer selected from 0 to 2,
Ar₁, Ar₃, and Ar₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₁ may be C(R₁₅)(R₁₆), N(R₁₅), O, or S,
R₁ to R₈, R₁₀ to R₁₆, R₂₀, and R₃₀ may each independently be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b1 may be an integer selected from 0 to 3,
b2 may be an integer selected from 0 to 6,
b3 may be an integer selected from 0 to 5,
R₁₀ₐ may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₆₀ carbocyclic group, a C₁-C₆₀ heterocyclic group, a C₆-C₆₀ aryloxy group, or a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂), and
Q₁ to Q₃, Q₁₁ to Q₁₃, Q₂₁ to Q₂₃, and Q₃₁ to Q₃₃ may each independently be:
hydrogen; deuterium; —F; —C₁; —Br; —I; a hydroxyl group; a cyano group; a nitro group; a C₁-C₆₀ alkyl group; a C₂-C₆₀ alkenyl group; a C₂-C₆₀ alkynyl group; a C₁-C₆₀ alkoxy group; or
a C₃-C₆₀ carbocyclic group or a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

In one or more embodiments, the emission layer may include the first compound and the second compound. The first compound and the second compound may each be a host. The emission layer may be to emit (e.g., configured to emit) blue light.

In one or more embodiments, the emission layer may include a first emission layer and a second emission layer arranged between the first emission layer and the second electrode. The first emission layer may be on (e.g., in direct contact with) the second emission layer. The first emission layer may include the first compound, and the second emission layer may include the second compound.

In one or more embodiments, the emission layer may include the first emission layer, the second emission layer, and the third emission layer, which are stacked in the stated order. In one or more embodiments, the emission layer may include the first emission layer, the second emission layer arranged between the first emission layer and the second electrode, and the third emission layer arranged between the second emission layer and the second electrode. The second emission layer may be on (e.g., in direct contact with) each of the first emission layer and the third emission layer. The first emission layer and the third emission layer may include the second compound, and the second emission layer may include the first compound.

In one or more embodiments, the interlayer may further include a hole transport region arranged between the first electrode and the emission layer, and the hole transport region may include the third compound. In one or more embodiments, the hole transport region may include: an electron blocking layer; and a hole injection layer, a hole transport layer, an emission auxiliary layer, or any combination thereof, and the electron blocking layer may include the third compound. The electron blocking layer may be on (e.g., in direct contact with) the emission layer. When the emission layer includes the first emission layer and the second emission layer, which are arranged in the stated order, the electron blocking layer may be on (e.g., in direct contact with) the first emission layer closest to the hole transport region.

In one or more embodiments, the interlayer may further include a fourth compound containing boron. In one or more embodiments, the emission layer may include the fourth compound. The fourth compound may be a dopant. The amount of the fourth compound in the emission layer may be smaller than the amount of the first compound and/or the amount of the second compound.

In one or more embodiments, the first compound and the second compound may each independently include at least two deuteriums, at least three deuteriums, or at least four deuteriums. In one or more embodiments, each of the first compound and the second compound may not include (e.g., may exclude) hydrogen.

In one or more embodiments, in Formula 1, each of a group represented by (L₁)ₐ₁ and a group represented by (L₂)ₐ₂ may include deuterium. Each of the group represented by (L₁)ₐ₁ and the group represented by (L₂)ₐ₂ may include at least two deuteriums, at least three deuteriums, or at least four deteriums. In one or more embodiments, each of the group represented by (L₁)ₐ₁ and the group represented by (L₂)ₐ₂ may not include (e.g., may exclude) hydrogen.

In Formula 1, each of L₁ and L₂ may be a C₆-C₆₀ arylene group that is unsubstituted or substituted with at least one R₁₀ₐ. In one or more embodiments, L₁ and L₂ may each independently be a benzene group, a naphthalene group, an anthracene group, a phenanthrene group, a perylene group, a phenalene group, a pyrene group, or a chrysene group, each unsubstituted or substituted with at least one R₁₀ₐ. In more detail, L₁ and L₂ may each independently be a benzene group or a naphthalene group, each unsubstituted or substituted with at least one deuterium.

In one or more embodiments, in Formula 1, L₁ and L₂ may each independently be represented by at least one of (e.g., by at least one selected from among) Formulae LK1 to LK13:

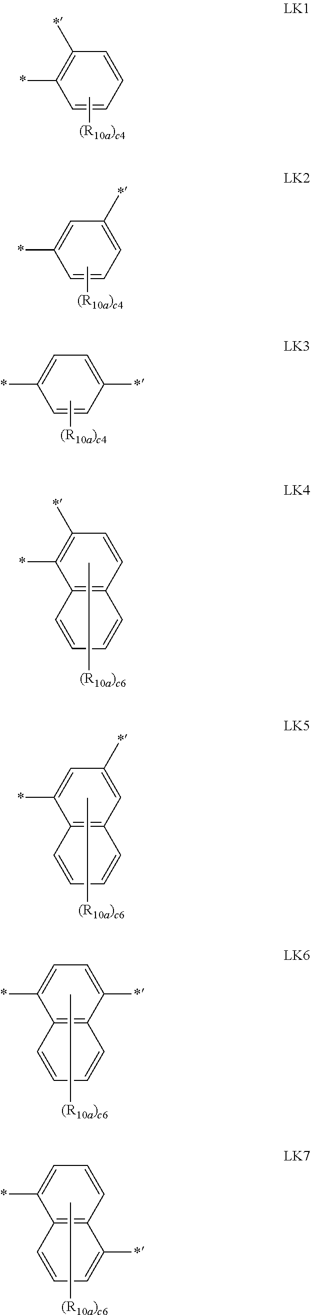

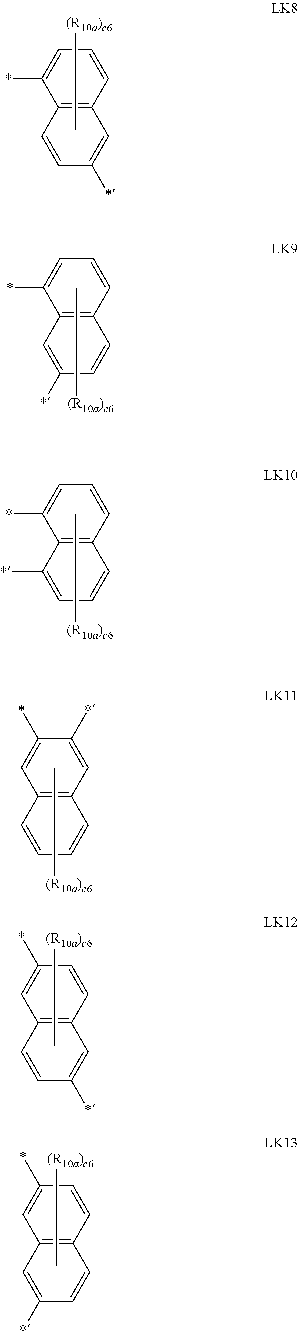

wherein, in Formulae LK1 to LK13,
c4 may be an integer selected from 0 to 4, and c6 may be an integer selected from 0 to 6, and
* and *′ each indicate a binding site to a neighboring atom.

In one or more embodiments, in Formulae LK1 to LK13, at least one of R₁₀ₐ in the number of c4 may be deuterium, and at least one of R₁₀ₐ in the number of c6 may be deuterium.

In one or more embodiments, in Formula 1, a group represented by Ar may include deuterium. The group represented by Ar may include at least two deuteriums, at least three deuteriums, or at least four deuteriums. In one or more embodiments, the group represented by Ar may not include (e.g., may exclude) hydrogen.

In one or more embodiments, in Formula 1, Ar may be a C₆-C₆₀ aryl group that is unsubstituted or substituted with at least one R₁₀ₐ. In one or more embodiments, Ar may be a phenyl group, a naphthyl group, an anthracenyl group, a phenanthrenyl group, a perylenyl group, a phenalenyl group, a pyrenyl group, or a chrysenyl group, each unsubstituted or substituted with at least one R₁₀ₐ. In more detail, Ar may be a phenyl group or a naphthyl group, each unsubstituted or substituted with at least one deuterium.

In one or more embodiments, in Formula 1, at least one of (e.g., selected from among) R₁ to R₈ may be deuterium. In one or more embodiments, at least two of (e.g., selected from among) R₁ to R₈ may be deuteriums, at least three of R₁ to R₈ may be deuteriums, at least four of R₁ to R₈ may be deuteriums, at least five of R₁ to R₈ may be deuteriums, at least six of R₁ to R₈ may be deuteriums, at least seven of R₁ to R₈ may be deuteriums, or all of R₁ to R₈ may be deuteriums.

In Formula 1, at least one of R₁₀ in the number of b1 may be deuterium. In Formula 1, at least one of R₁₁ to R₁₄ may be deuterium. In one or more embodiments, at least one of R₁₀ to R₁₄ may be deuterium, at least two of R₁₀ to R₁₄ may be deuteriums, at least three of R₁₀ to R₁₄ may be deuteriums, at least four of R₁₀ to R₁₄ may be deuteriums, or all of R₁₀ to R₁₄ may be deuteriums.

In Formula 1, at least one of R₁₀ in the number of b1 may be a phenyl group that is unsubstituted or substituted with at least one deuterium. In Formula 1, at least one of R₁₁ to R₁₄ may be a phenyl group that is unsubstituted or substituted with at least one deuterium.

R₁₀, R₁₁, R₁₂, R₁₃, and R₁₄ may each independently be hydrogen, deuterium, a phenyl group, or a phenyl group that is substituted with deuterium. When R₁₀, R₁₁, R₁₂, R₁₃, or R₁₄ are each a phenyl group that is substituted with deuterium, the phenyl group may be substituted with one deuterium, two deuteriums, three deuteriums, four deuteriums, or five deuteriums.

In one or more embodiments, in Formula 1, X₁ may be O or S.

In one or more embodiments, in Formula 1, X₁ may be C(R₁₅)(R₁₆) or N(R₁₅), and R₁₅ and R₁₆ may each independently be a C₆-C₆₀ aryl group that is unsubstituted or substituted with at least one R₁₀ₐ. In one or more embodiments, R₁₅ and R₁₆ may each independently be a phenyl group, a naphthyl group, an anthracenyl group, a phenanthrenyl group, a perylenyl group, a phenalenyl group, a pyrenyl group, or a chrysenyl group, each unsubstituted or substituted with at least one R₁₀ₐ. In more detail, R₁₅ and R₁₆ may each independently be a phenyl group or a naphthyl group, each unsubstituted or substituted with at least one deuterium.

In one or more embodiments, in Formula 1, a1 may be 0 or 1, and a2 may be 0 or 1. The sum of a1 and a2 may be 0 or 1. When a1 is 0, (L₁)ₐ₁ may refer to a single bond, and when a2 is 0, (L₂)ₐ₂ may refer to a single bond.

In one or more embodiments, the first compound and the second compound may each independently be represented by at least one of Formulae 1-1 to 1-4:

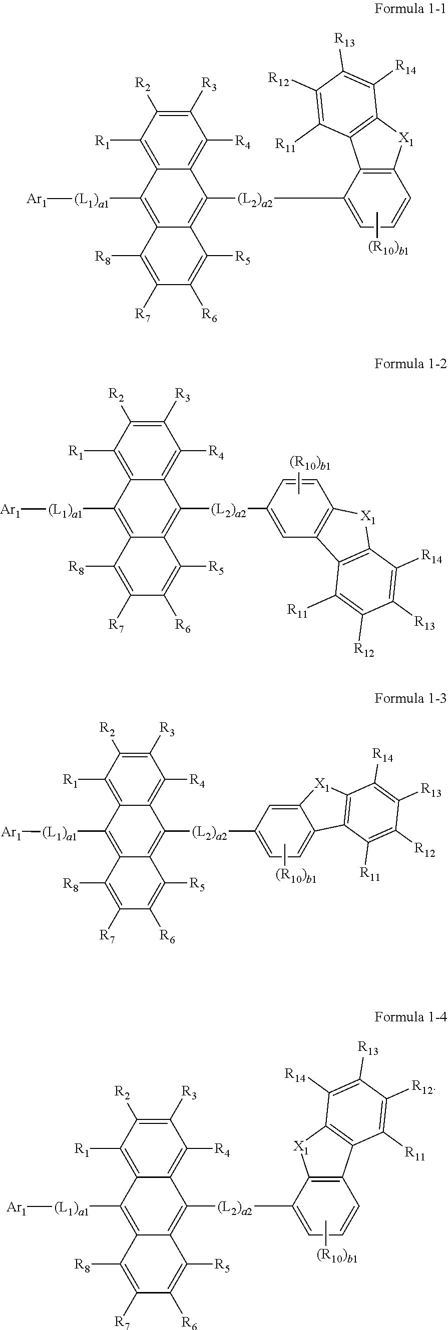

In Formulae 1-1 to 1-4, L₁, L₂, a1, a2, Ar₁, X₁, R₁ to R₈, R₁₀ to R₁₄, and b1 are each as described herein.

In one or more embodiments, at least one of the first compound or the second compound may satisfy at least one of Conditions 1 to 6:

### Condition 1

Ar₁ is a C₃-C₆₀ carbocyclic group that is substituted with at least one deuterium or a C₁-C₆₀ heterocyclic group that is unsubstituted or substituted with at least one deuterium;

### Condition 2

L₁ is a C₃-C₆₀ carbocyclic group that is substituted with at least one deuterium or a C₁-C₆₀ heterocyclic group that is unsubstituted or substituted with at least one deuterium;

### Condition 3

at least one of R₁ to R₈ is deuterium;

### Condition 4

L₂ is a C₃-C₆₀ carbocyclic group that is substituted with at least one deuterium or a C₁-C₆₀ heterocyclic group that is unsubstituted or substituted with at least one deuterium;

### Condition 5

at least one R₁₀ (e.g., in the number of b1) is deuterium; and/or

### Condition 6

at least one of R₁₁ to R₁₄ is deuterium.

In one or more embodiments, each of the first compound and the second compound may satisfy at least one of Conditions 1 to 6.

At least one of the first compound and the second compound may satisfy at least two of Conditions 1 to 6, at least three of Conditions 1 to 6, at least four of Conditions 1 to 6, at least five of Conditions 1 to 6, or all of Conditions 1 to 6.

Herein, the expression “the first compound and the second compound are different from each other” may refer to that i) the number of deuteriums included in the first compound is different from the number of deuteriums included in the second compound, ii) the position of deuterium included in the first compound is different from the position of deuterium included in the second compound (for example, in the first compound, R₁ to R₈ in Formula 1 are deuteriums, whereas in the second compound, R₁₀ to R₁₄ in Formula 1 are deuteriums), iii) in the first compound, a2 in Formula 1 is 0, whereas in the second compound, a2 in Formula 1 is 1, or iv) the first compound is represented by Formula 1-2, whereas the second compound is represented by Formula 1-4.

In one or more embodiments, the first compound and the second compound may each independently be at least one compound selected from Compounds HH1 to HH11 in which at least one hydrogen is substituted with deuterium:

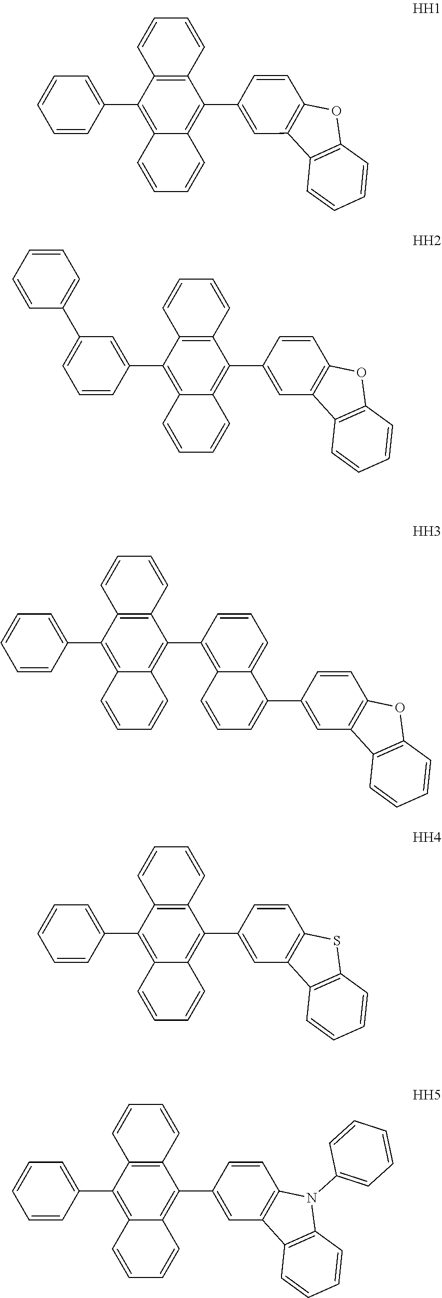 [SMILES: c1ccc(-c2c3ccccc3c(-c3ccc4oc5ccccc5c4c3)c3ccccc23)cc1 ; c1ccc(-c2cccc(-c3c4ccccc4c(-c4ccc5oc6ccccc6c5c4)c4ccccc34)c2)cc1 ; c1ccc(-c2c3ccccc3c(-c3cccc4c(-c5ccc6oc7ccccc7c6c5)cccc34)c3ccccc23)cc1 ; c1ccc(-c2c3ccccc3c(-c3ccc4sc5ccccc5c4c3)c3ccccc23)cc1 ; c1ccc(-c2c3ccccc3c(-c3ccc4c(c3)c3ccccc3n4-c3ccccc3)c3ccccc23)cc1]

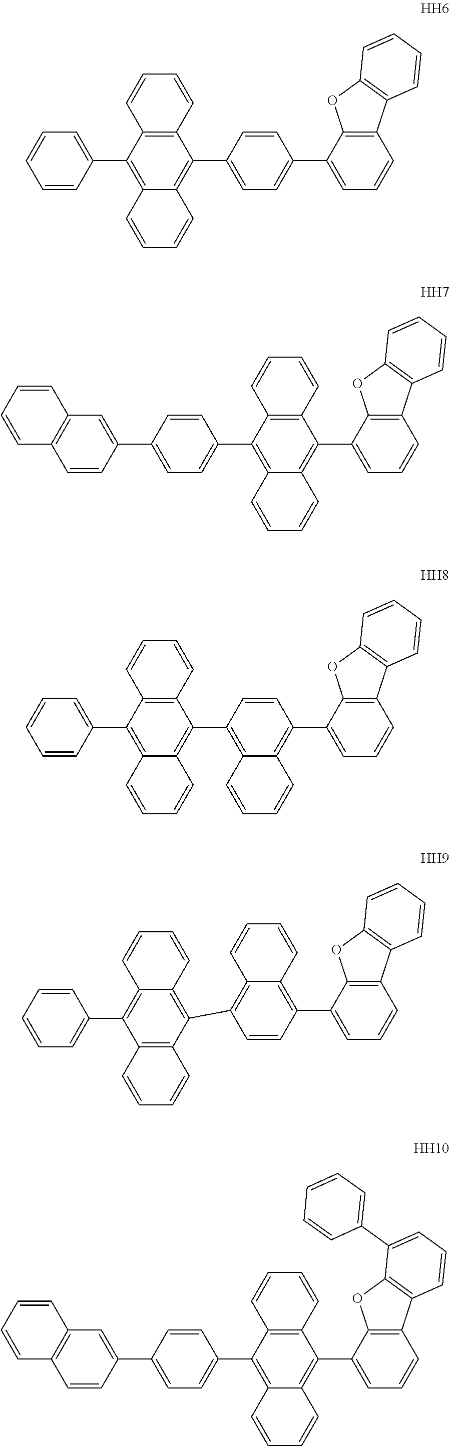 [SMILES: c1ccc(-c2c3ccccc3c(-c3ccc(-c4cccc5c4oc4ccccc45)cc3)c3ccccc23)cc1 ; c1ccc2cc(-c3ccc(-c4c5ccccc5c(-c5cccc6c5oc5ccccc56)c5ccccc45)cc3)ccc2c1 ; c1ccc(-c2c3ccccc3c(-c3ccc(-c4cccc5c4oc4ccccc45)c4ccccc34)c3ccccc23)cc1 ; c1ccc(-c2c3ccccc3c(-c3ccc(-c4cccc5c4oc4ccccc45)c4ccccc34)c3ccccc23)cc1 ; c1ccc(-c2cccc3c2oc2c(-c4c5ccccc5c(-c5ccc(-c6ccc7ccccc7c6)cc5)c5ccccc45)cccc23)cc1]

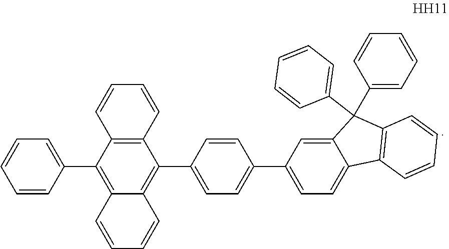 [SMILES: c1ccc(-c2c3ccccc3c(-c3ccc(-c4ccc5c(c4)C(c4ccccc4)(c4ccccc4)c4ccccc4-5)cc3)c3ccccc23)cc1]

In one or more embodiments, the first compound and the second compound may each independently selected from at least one of Compounds HD1 to HD76, and the first compound and the second compound may be different from each other:

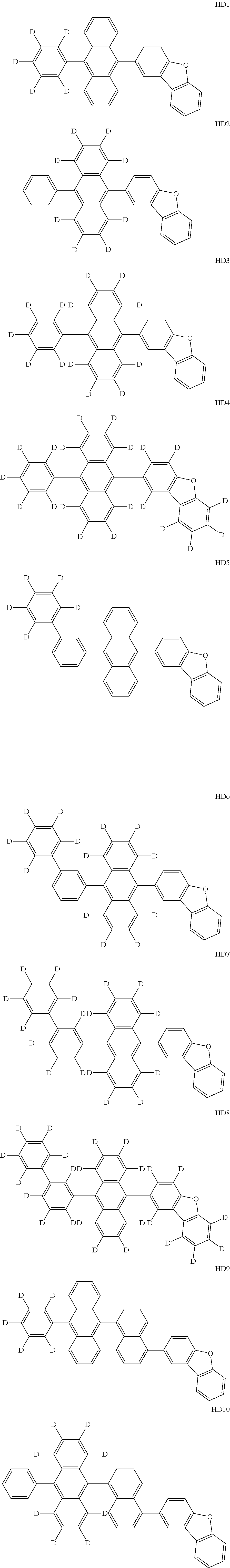

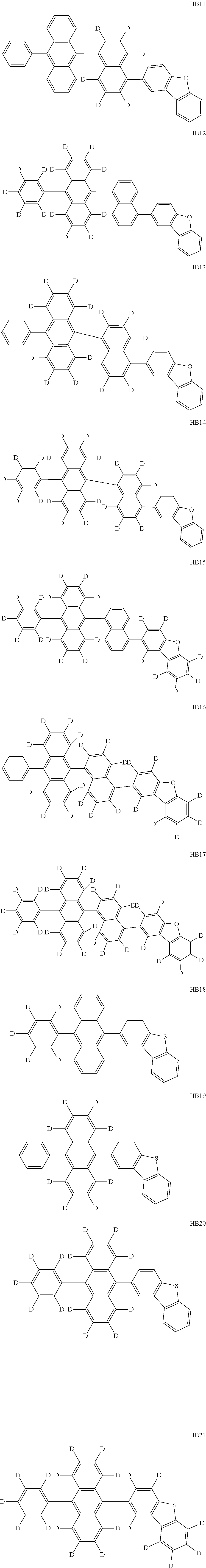

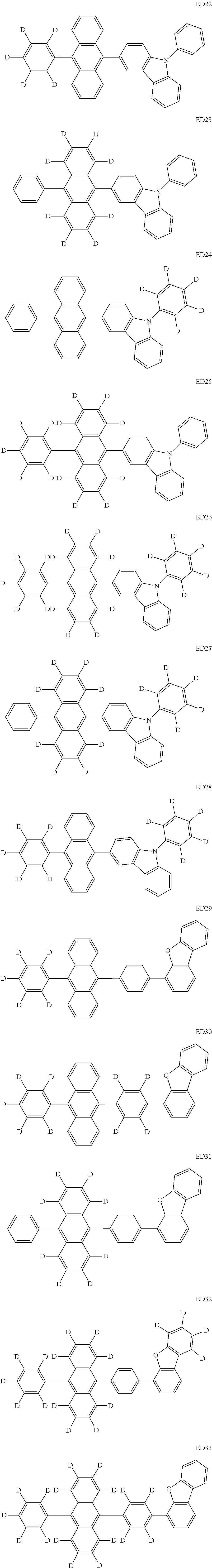

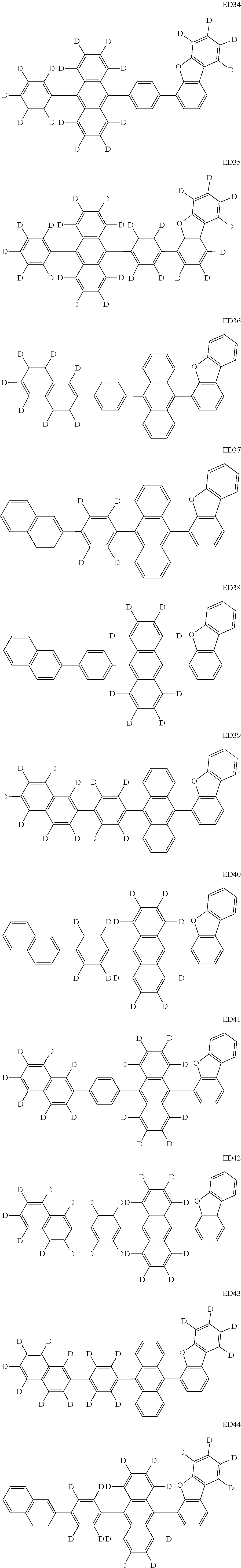

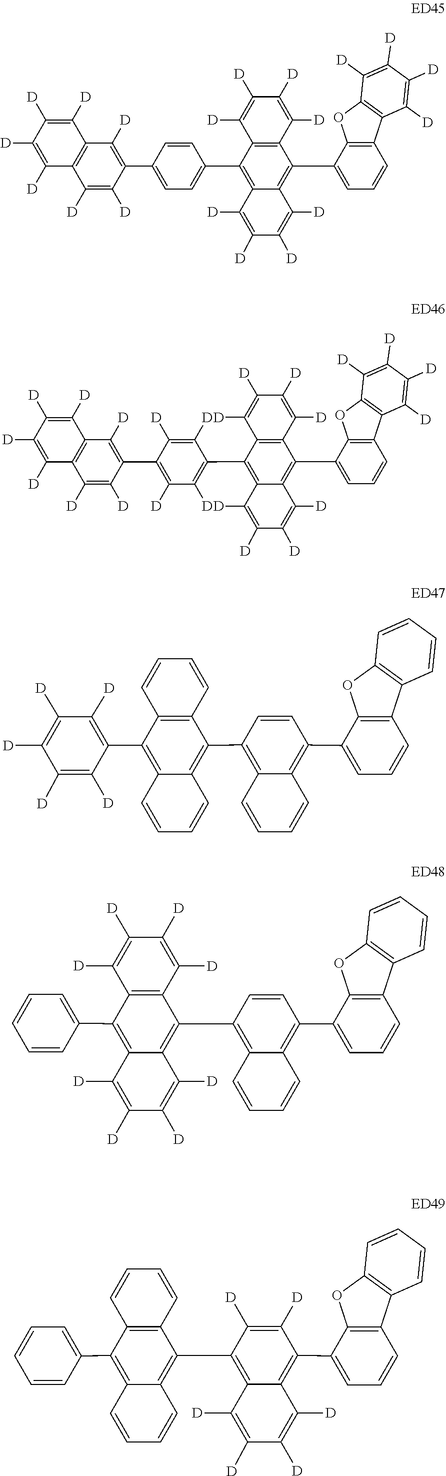

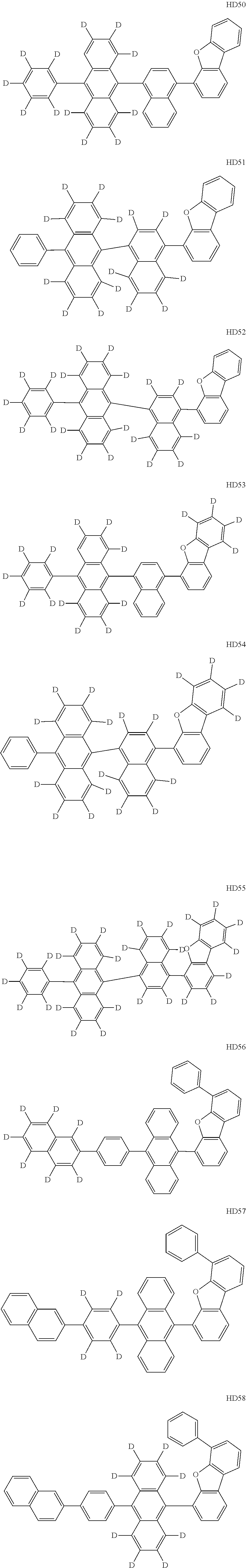

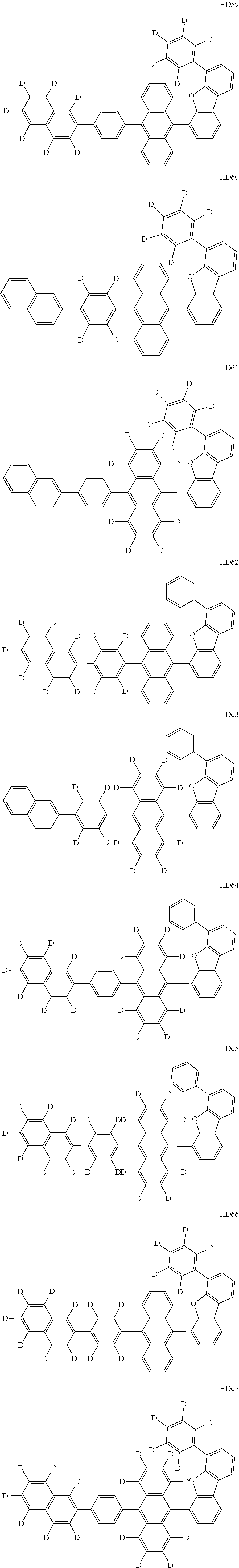

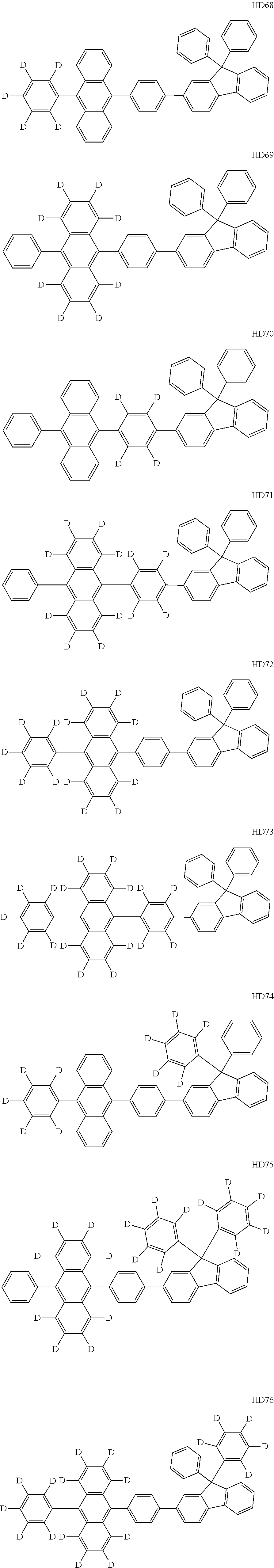

In one or more embodiments, in Formula 2, L₃ to L₅ may each independently be a benzene group, a naphthalene group, an anthracene group, a phenanthrene group, a perylene group, a phenalene group, a pyrene group, a chrysene group, a fluorene group, a carbazole group, a dibenzofuran group, a dibenzothiophene group, a benzofluorene group, a benzocarbazole group, a benzonaphthofuran group, or a benzonaphthothiophene group, each unsubstituted or substituted with at least one R₁₀ₐ. L₃ to L₅ may each independently be substituted with a C₁-C₆₀ alkyl group and/or a C₆-C₆₀ aryl group.

When a3 is 0, (L₃)ₐ₃ may be (e.g., refer to) a single bond, when a4 is 0, (L₄)ₐ₄ may be (e.g., refer to) a single bond, and when a5 is 0, (L₅)ₐ₅ may be (e.g., refer to) a single bond.

In one or more embodiments, in Formula 2, Ar₃ and Ar₄ may each independently be a benzene group, a naphthalene group, an anthracene group, a phenanthrene group, perylene group, a phenalene group, a pyrene group, a chrysene group, a fluorene group, a carbazole group, a dibenzofuran group, a dibenzothiophene group, a benzofluorene group, a benzocarbazole group, a benzonaphthofuran group, a benzonaphthothiophene group, a cyclopentane group, a cyclohexane group, a cycloheptane group, a cyclooctane group, an adamantane group, or a norbornane group, each unsubstituted or substituted with at least one R₁₀ₐ.

In one or more embodiments, in Formula 2, each of R₂₀ and R₃₀ may exclude (e.g., not be) —N(Q₁)(Q₂). In other words, the third compound represented by Formula 2 may include one amine group. In one or more embodiments, the third compound may include an amine group linked to a second carbon atom of a naphthalene core in Formula 2 and may not include (e.g., may exclude) an amine group linked to a first carbon atom of the naphthalene core.

In one or more embodiments, the third compound may be represented by at least one of Formulae 2-1 to 2-7:

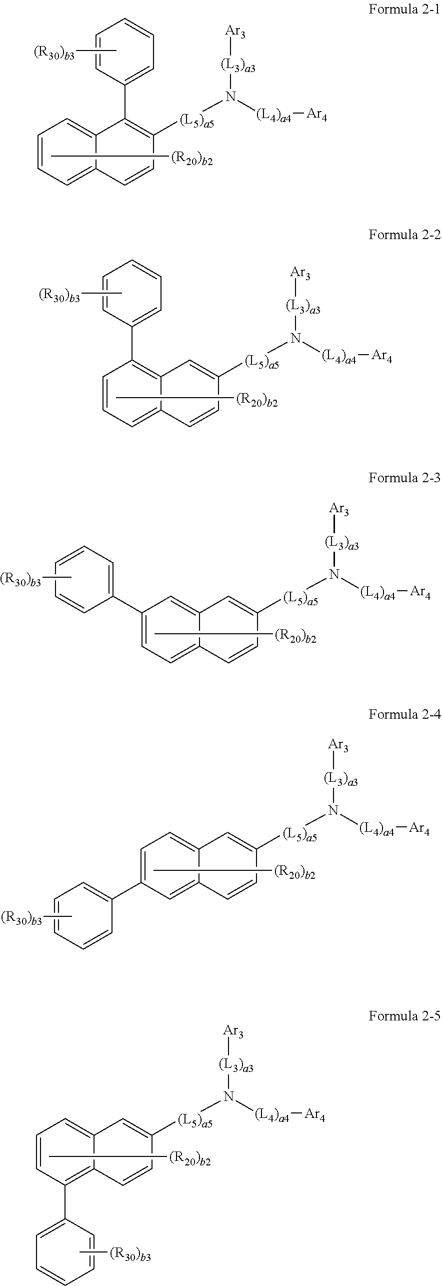

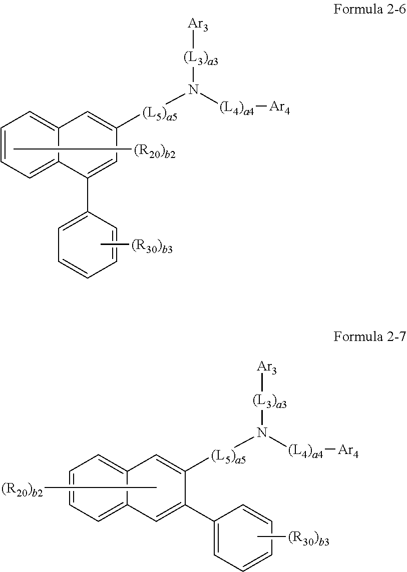

wherein, in Formulae 2-1 to 2-7, L₃ to L₅, a3 to a5, Ar₃, Ar₄, R₂₀, R₃₀, b2, and b3 may each be as described herein.

In one or more embodiments, the third compound may be at least one selected from Compounds EB1 to EB43

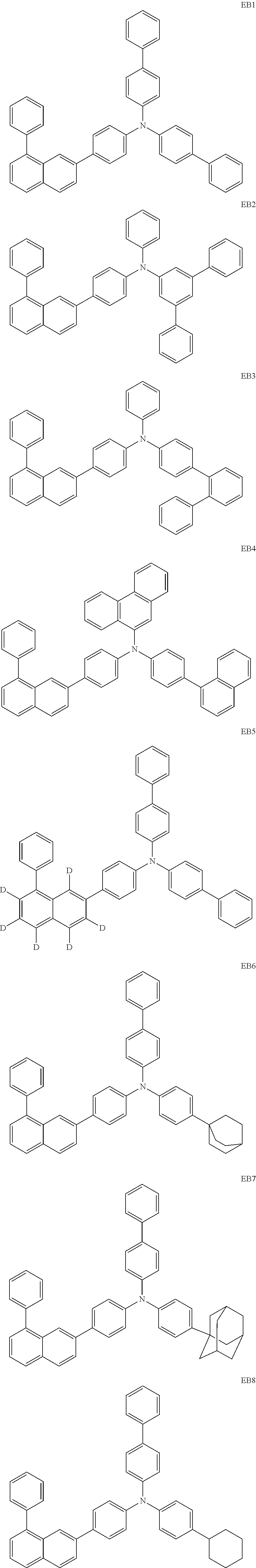

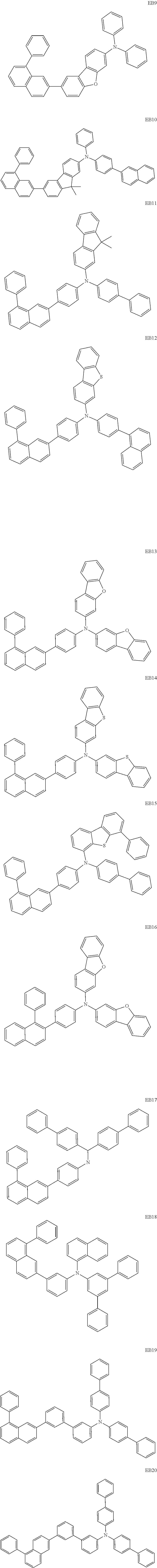

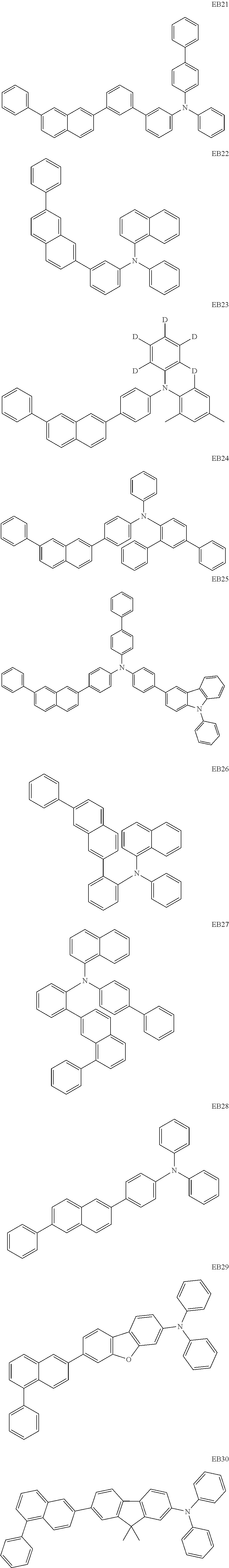

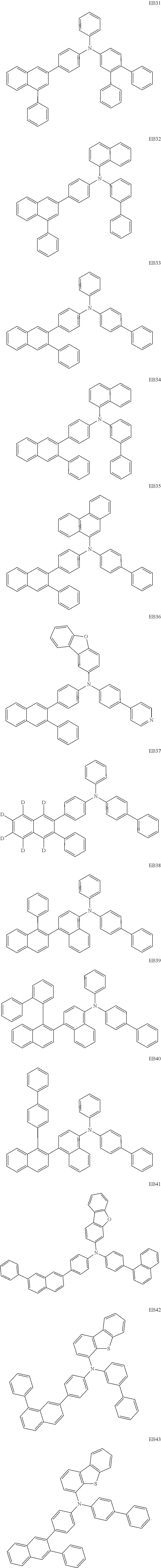

The first compound and the second compound may be different from each other, may each be represented by Formula 1, and may each include deuterium. In other words, the light-emitting device may include two or more different compounds selected from among compounds represented by Formula 1 and including deuterium. The light-emitting device including the two or more different compounds may have an improvement in at least one of driving voltage, luminescence efficiency, and lifespan, compared to a light-emitting device including one kind of (e.g., a single) host. In some embodiments, even when the light-emitting device has a multilayer emission layer structure by including, in the emission layer, the first emission layer including the first compound and the second emission layer including the second compound, the light-emitting device may have improved lifespan. By adjusting the position of deuterium included in each of the first compound and the second compound and the number of substituted deuteriums, and adjusting the relative amounts of the first compound and the second compound, the light-emitting device may have optimal or suitable driving voltage, optimal or suitable luminescence efficiency, and/or optimal or suitable lifespan.

In the third compound, an amine group may be linked to a second carbon atom of a naphthalene core, and a phenyl group may be substituted into (e.g., on) any carbon selected from among a first carbon atom and a third carbon atom to an eighth carbon atom of the naphthalene core. In other words, the third compound included in the light-emitting device may exclude (e.g., not be) a compound in which an amine group is linked to the first carbon atom of the naphthalene core, and exclude (e.g., nor be) a compound in which a phenyl group is not substituted into (e.g., on) the naphthalene core. The light-emitting device may have further improved driving voltage, further improved luminescence efficiency, and/or further improved lifespan by including the third compound in a layer adjacent to the emission layer.

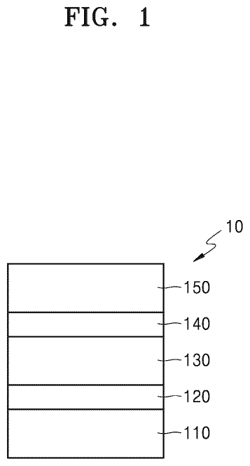

### Description of FIG. 1

FIG. 1 is a schematic cross-sectional view of a light-emitting device 10 according to one or more embodiments. The light-emitting device 10 includes a first electrode 110, an interlayer, and a second electrode 150. The interlayer may include a hole transport region 120, an emission layer 130, and an electron transport region 140.

Hereinafter, a structure of the light-emitting device 10 according to one or more embodiments and a method of manufacturing the light-emitting device 10 are described with reference to FIG. 1.

### Firstelectrode 110

In FIG. 1, a substrate may be additionally arranged under the first electrode 110 or on (e.g., above) the second electrode 150. As the substrate, a glass substrate or a plastic substrate may be utilized. The substrate may be a flexible substrate. For example, the substrate may include plastics with excellent or suitable heat resistance and durability, such as polyimide, polyethylene terephthalate (PET), polycarbonate, polyethylene naphthalate, polyarylate (PAR), polyetherimide, or any combination thereof.

The first electrode 110 may be formed by providing a material for forming the first electrode 110 on the substrate by utilizing a deposition method or a sputtering method. When the first electrode 110 is an anode, a material for forming the first electrode 110 may be a high work-function material that facilitates injection of holes. The “term “high work-function material” as utilized herein refers to a substance (e.g., a metal or metal alloy) that requires a relatively high amount of energy to emit electrons from its surface.

The first electrode 110 may be a reflective electrode, a semi-transmissive electrode, or a transmissive electrode. When the first electrode 110 is a transmissive electrode, a material for forming the first electrode 110 may include indium tin oxide (ITO), indium zinc oxide (IZO), tin oxide (SnO₂), zinc oxide (ZnO), or any combination thereof. In one or more embodiments, when the first electrode 110 is a semi-transmissive electrode or a reflective electrode, a material for forming the first electrode 110 may include magnesium (Mg), silver (Ag), aluminum (Al), aluminum-lithium (Al—Li), calcium (Ca), magnesium-indium (Mg—In), magnesium-silver (Mg—Ag), or any combination thereof.

The first electrode 110 may have a single-layer structure consisting of a single layer or a multilayer structure including multiple layers. In one or more embodiments, the first electrode 110 may have a three-layer structure of ITO/Ag/ITO.

### Interlayer

The interlayer may be provided on the first electrode 110. The interlayer may include a hole transport region 120, an emission layer 130, and an electron transport region 140.

The interlayer may include one or more suitable organic materials, a metal-containing compound such as an organometallic compound, an inorganic material such as quantum dots, and/or the like.

In one or more embodiments, the interlayer may include i) two or more emitting units sequentially stacked between the first electrode 110 and the second electrode 150, and ii) a charge generation layer between every two emitting units. When the interlayer includes the emitting units and the charge generation layer, the light-emitting device 10 may be a tandem light-emitting device.

### Hole Transport Region 120

The hole transport region 120 may have i) a single-layer structure including (e.g., consisting of) a single layer including a single material, ii) a single-layer structure including (e.g., consisting of) a single layer including multiple different materials, or iii) a multilayer structure including (e.g., consisting of) multiple layers including multiple different materials.

The hole transport region 120 may include a hole injection layer, a hole transport layer, an emission auxiliary layer, an electron blocking layer, or any combination thereof.

For example, the hole transport region 120 may have a multilayer structure including a hole injection layer/hole transport layer structure, a hole injection layer/hole transport layer/emission auxiliary layer structure, a hole injection layer/emission auxiliary layer structure, a hole transport layer/emission auxiliary layer structure, or a hole injection layer/hole transport layer/electron blocking layer structure. In one or more embodiments, wherein constituent layers of each structure are stacked in the stated order from the first electrode 110.

The hole transport region may include a compound represented by Formula 201, a compound represented by Formula 202, or any combination thereof:

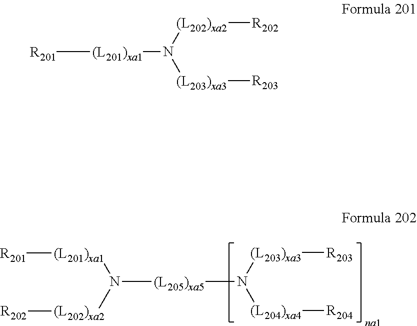

wherein, in Formulae 201 and 202,
L₂₀₁ to L₂₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
L₂₀₅ may be *—O—*′, *—S—*′, *—N(Q₂₀₁)-*′, a C₁-C₂₀ alkylene group that is unsubstituted or substituted with at least one R₁₀ₐ, a C₂-C₂₀ alkenylene group that is unsubstituted or substituted with at least one R₁₀ₐ, a C₃-C₆₀ carbocyclic group that is unsubstituted or substituted with at least one R₁₀ₐ, or a C₁-C₆₀ heterocyclic group that is unsubstituted or substituted with at least one R₁₀ₐ,
xa1 to xa4 may each independently be an integer selected from 0 to 5,
xa5 may be an integer selected from 1 to 10,
R₂₀₁ to R₂₀₄ and Q₂₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
R₂₀₁ and R₂₀₂ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group (for example, a carbazole group and/or the like) that is unsubstituted or substituted with at least one R₁₀ₐ (for example, Compound HT16),
R₂₀₃ and R₂₀₄ may optionally be linked to each other via a single bond, a C₁-C₅ alkylene group that is unsubstituted or substituted with at least one R₁₀ₐ, or a C₂-C₅ alkenylene group that is unsubstituted or substituted with at least one R₁₀ₐ to form a C₈-C₆₀ polycyclic group that is unsubstituted or substituted with at least one R₁₀ₐ, and
na1 may be an integer selected from 1 to 4.

In one or more embodiments, each of Formulae 201 and 202 may include at least one of groups represented by Formulae CY201 to CY217:

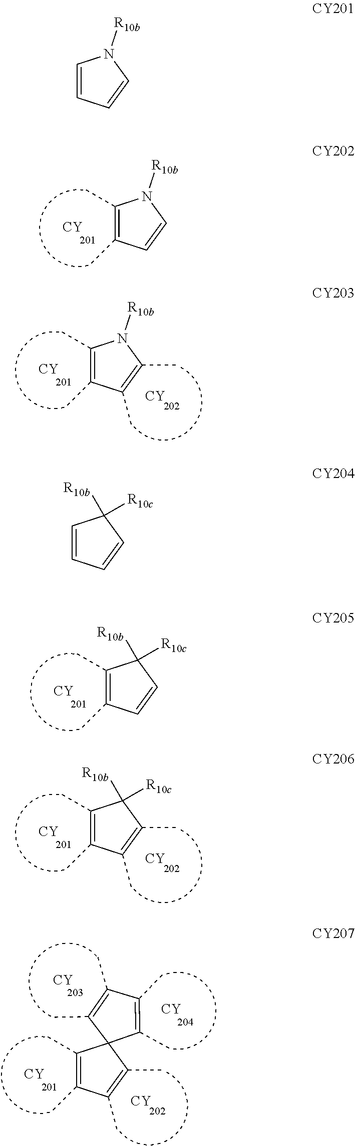

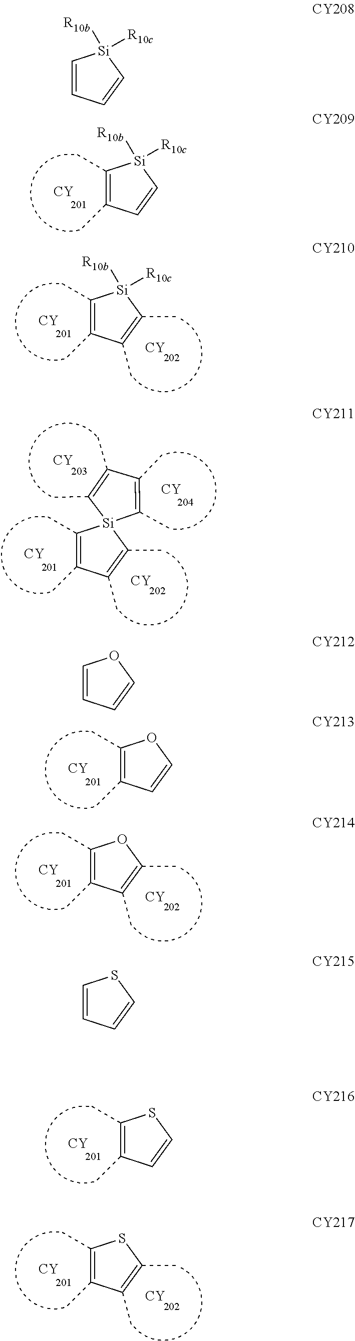

In Formulae CY201 to CY217, R_{10b} and R_{10c} may each be as described in connection with R₁₀ₐ, ring CY201 to ring CY₂O₄ may each independently be a C₃-C₂₀ carbocyclic group or a C₁-C₂₀ heterocyclic group, and at least one hydrogen in Formulae CY201 to CY217 may be unsubstituted or substituted with R₁₀ₐ as described herein.

In one or more embodiments, ring CY201 to ring CY204 in Formulae CY201 to CY217 may each independently be a benzene group, a naphthalene group, a phenanthrene group, or an anthracene group.

In one or more embodiments, each of Formulae 201 and 202 may include at least one of the groups represented by Formulae CY201 to CY203.

In one or more embodiments, Formula 201 may include at least one of the groups represented by Formulae CY201 to CY203 and at least one of the groups represented by Formulae CY204 to CY217.

In one or more embodiments, in Formula 201, xa1 may be 1, R₂₀₁ may be a group represented by one of Formulae CY201 to CY203, xa2 may be 0, and R₂₀₂ may be a group represented by one of Formulae CY204 to CY207.

In one or more embodiments, each of Formulae 201 and 202 may not include (e.g., may exclude) the groups represented by Formulae CY201 to CY203.

In one or more embodiments, each of Formulae 201 and 202 may not include (e.g., may exclude) the groups represented by Formulae CY201 to CY203 and may include at least one of the groups represented by Formulae CY204 to CY217.

In one or more embodiments, each of Formulae 201 and 202 may not include (e.g., may exclude) the groups represented by Formulae CY201 to CY217.

In one or more embodiments, the hole transport region may include: at least one of Compounds HT1 to HT46; m-MTDATA; TDATA; 2-TNATA; NPB(NPD); p-NPB; TPD; spiro-TPD; spiro-NPB; methylated NPB; TAPC; HMTPD; 4,4′, 4″-tris(N-carbazolyl)triphenylamine (TCTA); polyaniline/dodecylbenzenesulfonic acid (PANI/DBSA); poly(3,4-ethylenedioxythiophene)/poly(4-styrenesulfonate) (PEDOT/PSS); polyaniline/camphor sulfonic acid (PANI/CSA); polyaniline/poly(4-styrenesulfonate) (PANI/PSS); or any combination thereof:

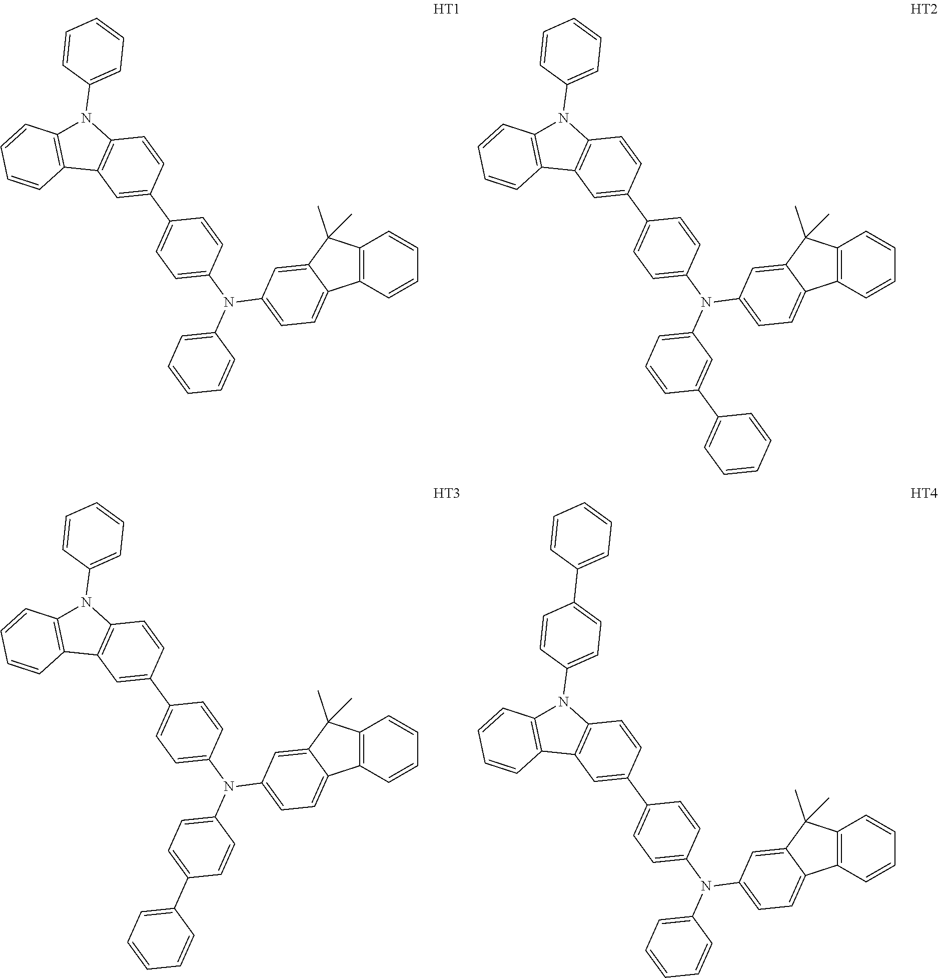 [SMILES: CC1(C)c2ccccc2-c2ccc(N(c3ccccc3)c3ccc(-c4ccc5c(c4)c4ccccc4n5-c4ccccc4)cc3)cc21 ; CC1(C)c2ccccc2-c2ccc(N(c3ccc(-c4ccc5c(c4)c4ccccc4n5-c4ccccc4)cc3)c3cccc(-c4ccccc4)c3)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; CC1(C)c2ccccc2-c2ccc(N(c3ccccc3)c3ccc(-c4ccc5c(c4)c4ccccc4n5-c4ccc(-c5ccccc5)cc4)cc3)cc21]

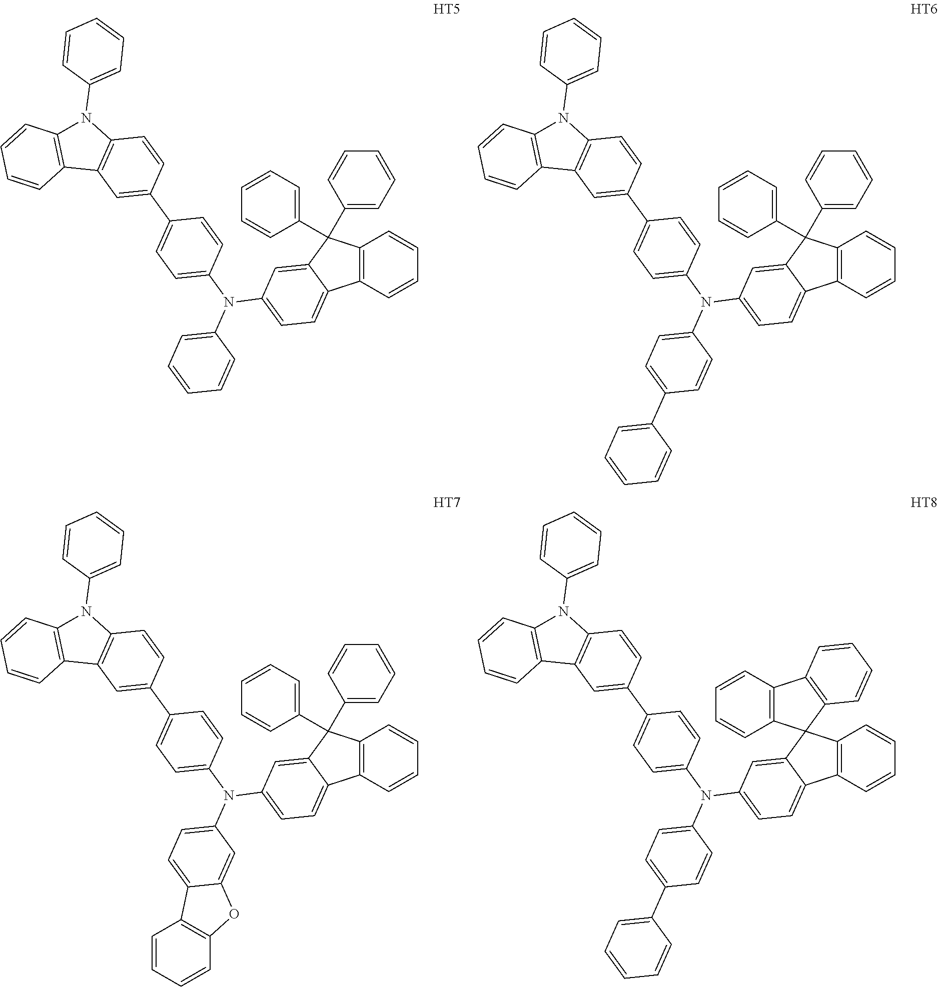 [SMILES: c1ccc(N(c2ccc(-c3ccc4c(c3)c3ccccc3n4-c3ccccc3)cc2)c2ccc3c(c2)C(c2ccccc2)(c2ccccc2)c2ccccc2-3)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; c1ccc(-n2c3ccccc3c3cc(-c4ccc(N(c5ccc6c(c5)C(c5ccccc5)(c5ccccc5)c5ccccc5-6)c5ccc6c(c5)oc5ccccc56)cc4)ccc32)cc1 ; c1ccc(-c2ccc(N(c3ccc(-c4ccc5c(c4)c4ccccc4n5-c4ccccc4)cc3)c3ccc4c(c3)C3(c5ccccc5-c5ccccc53)c3ccccc3-4)cc2)cc1]

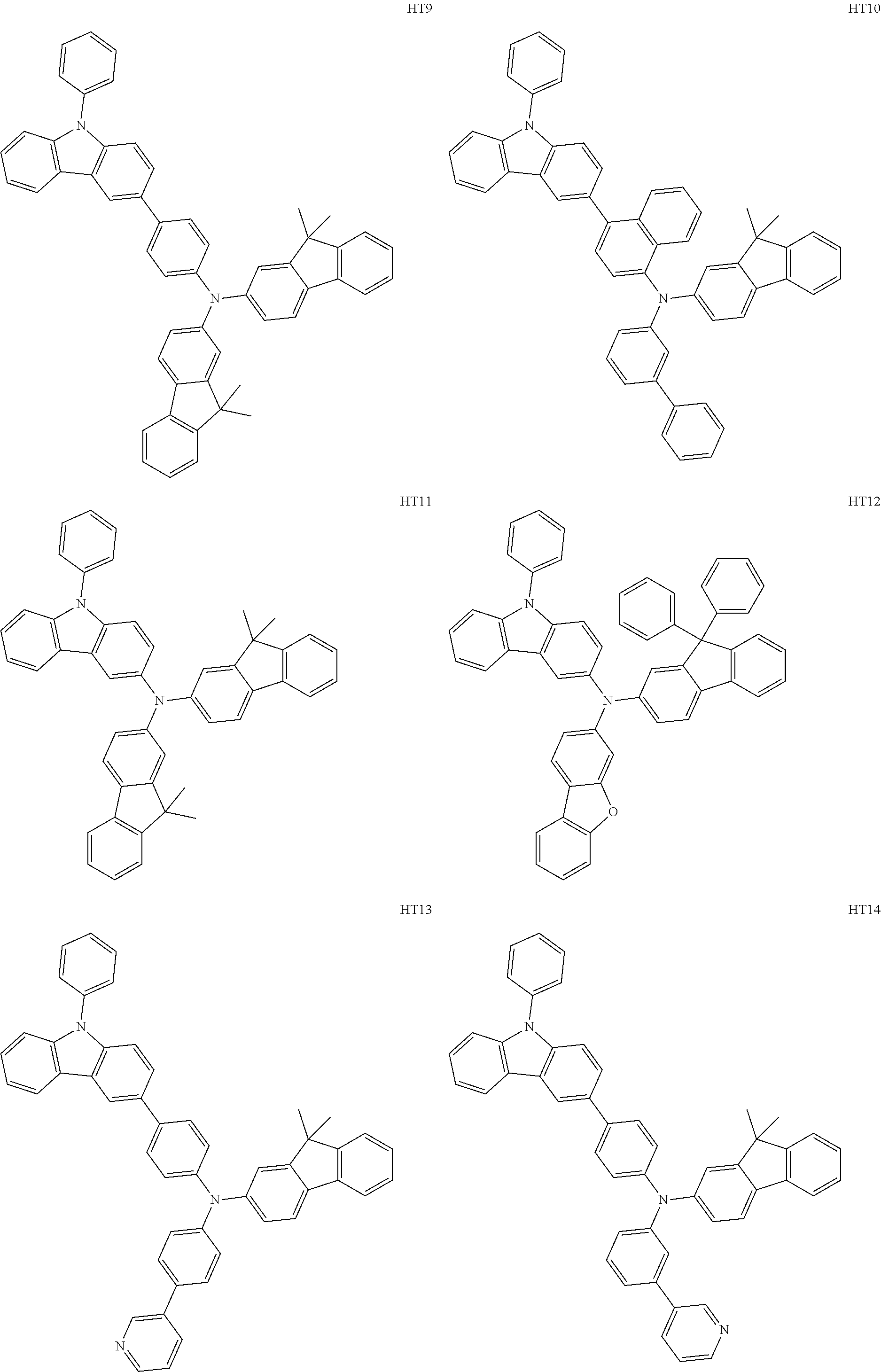

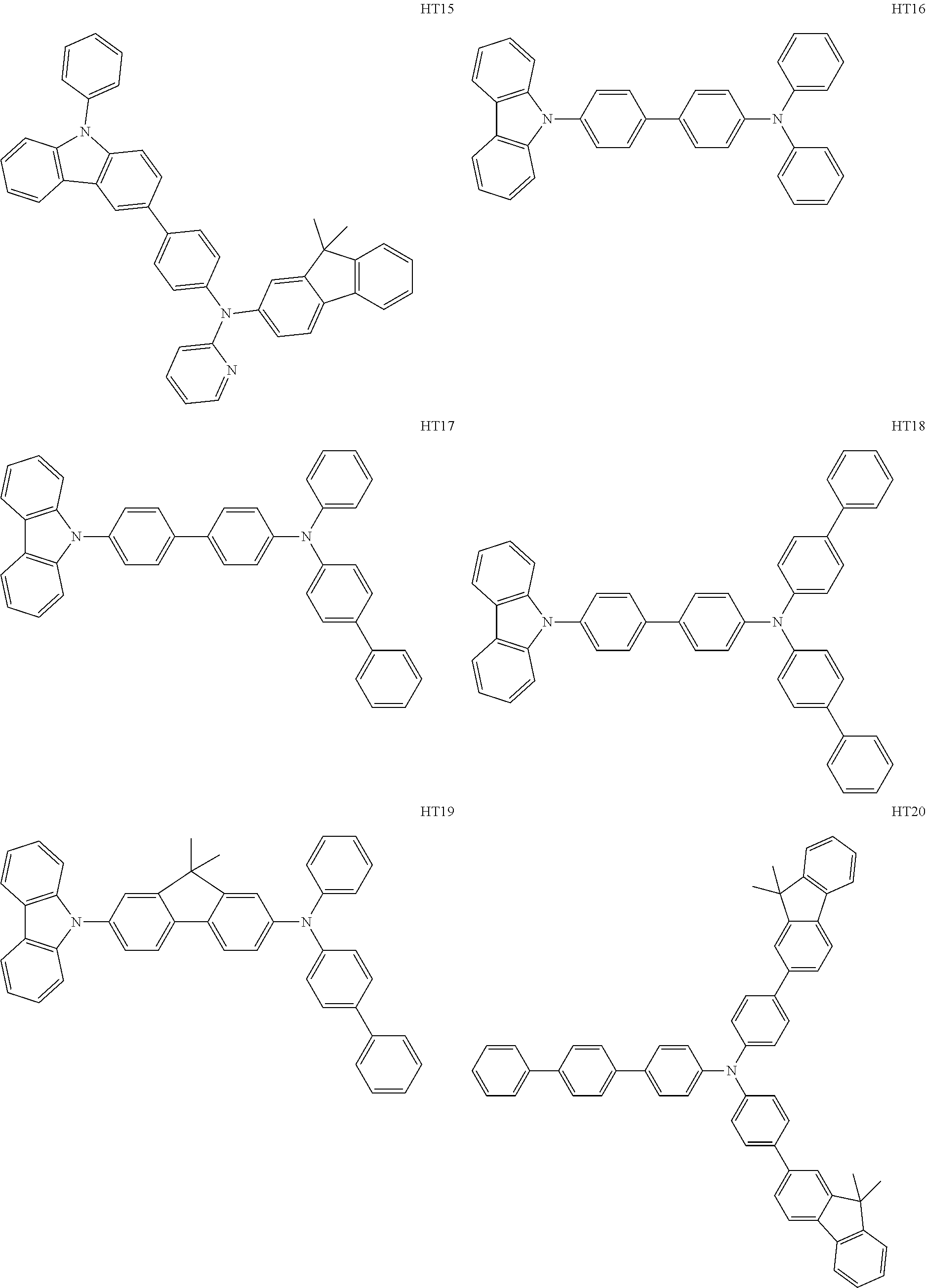

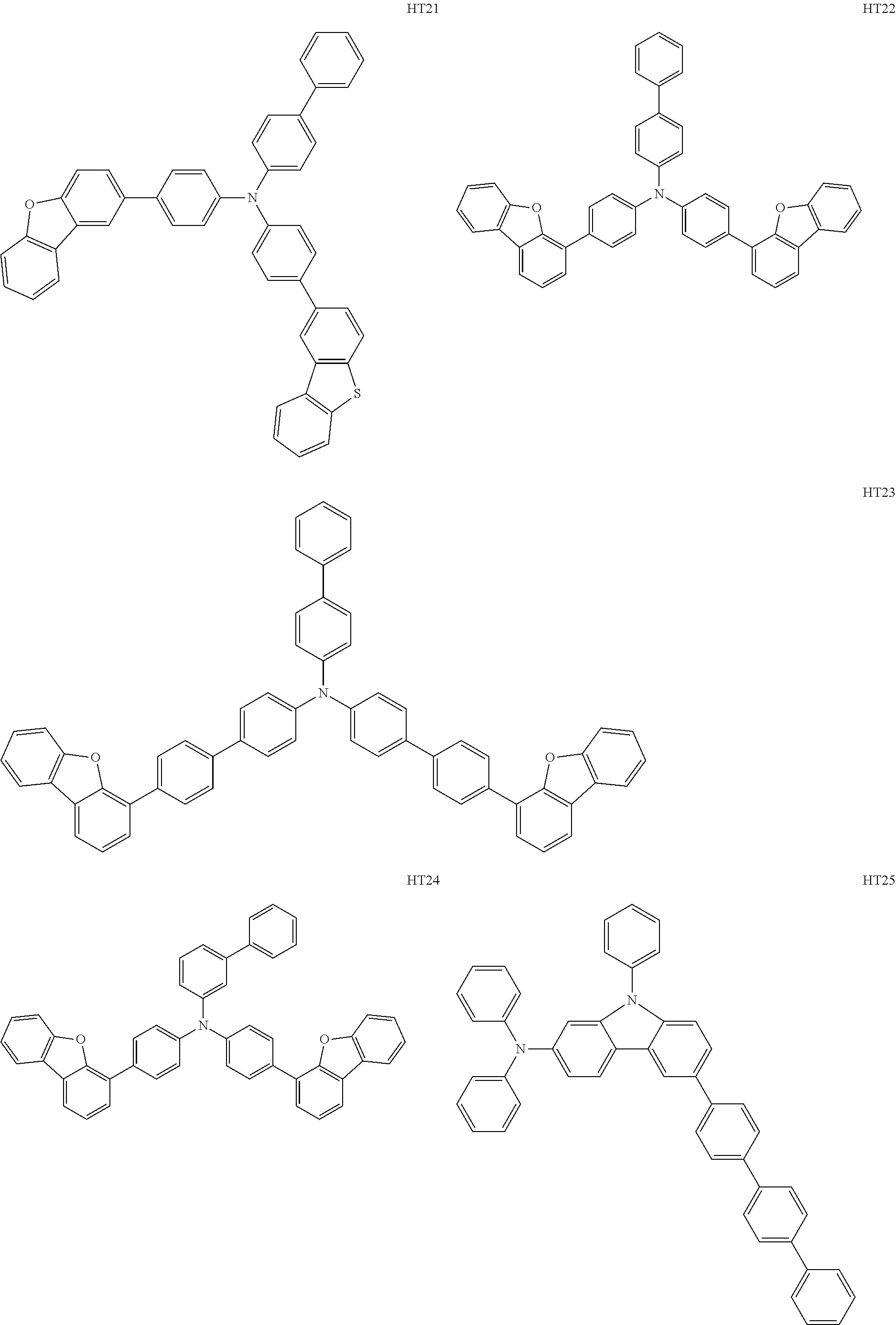 [SMILES: c1ccc(-c2ccc(N(c3ccc(-c4ccc5oc6ccccc6c5c4)cc3)c3ccc(-c4ccc5sc6ccccc6c5c4)cc3)cc2)cc1 ; c1ccc(-c2ccc(N(c3ccc(-c4cccc5c4oc4ccccc45)cc3)c3ccc(-c4cccc5c4oc4ccccc45)cc3)cc2)cc1 ; c1ccc(-c2ccc(N(c3ccc(-c4ccc(-c5cccc6c5oc5ccccc56)cc4)cc3)c3ccc(-c4ccc(-c5cccc6c5oc5ccccc56)cc4)cc3)cc2)cc1 ; c1ccc(-c2cccc(N(c3ccc(-c4cccc5c4oc4ccccc45)cc3)c3ccc(-c4cccc5c4oc4ccccc45)cc3)c2)cc1 ; c1ccc(-c2ccc(-c3ccc(-c4ccc5c(c4)c4ccc(N(c6ccccc6)c6ccccc6)cc4n5-c4ccccc4)cc3)cc2)cc1]

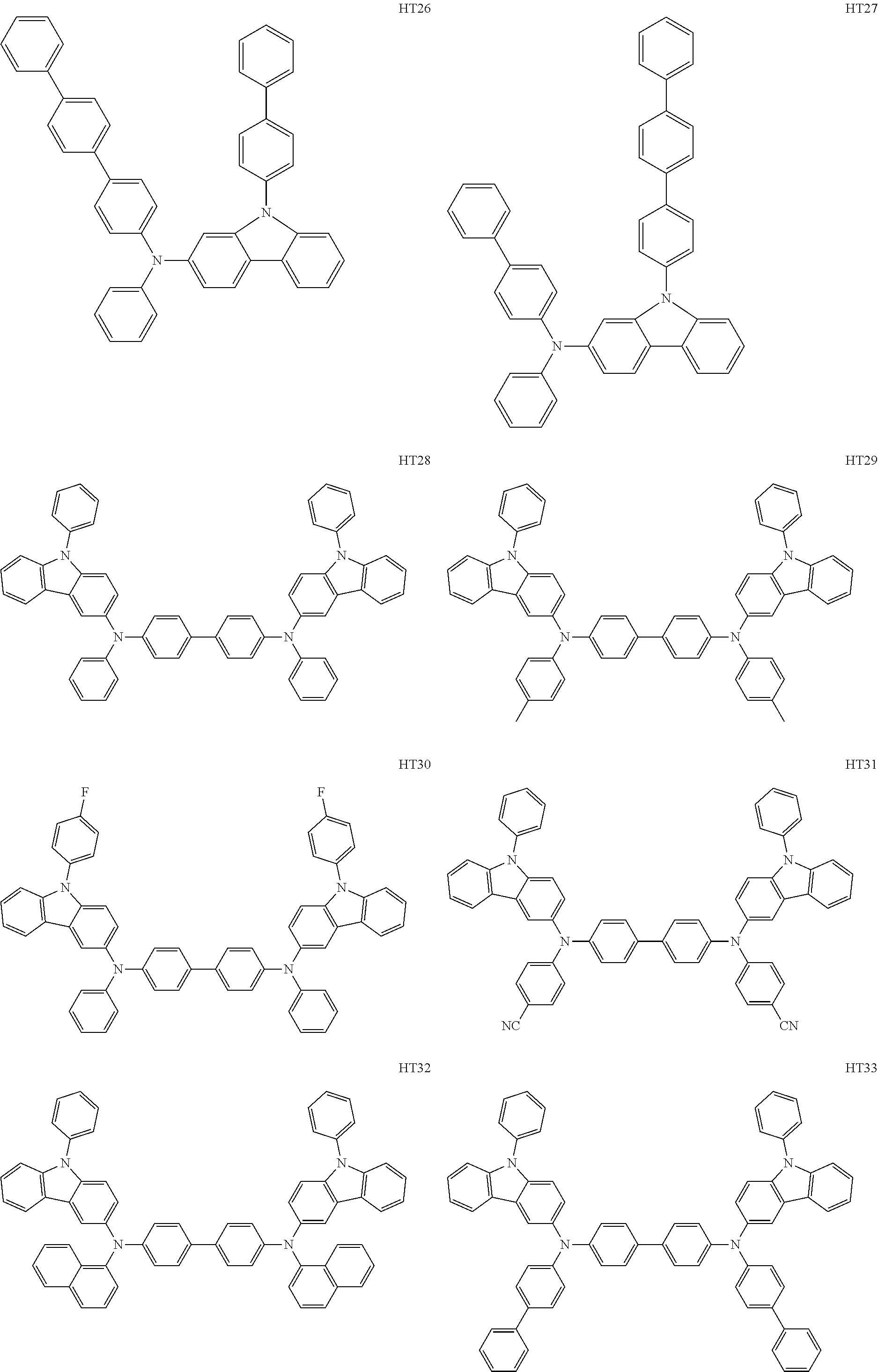

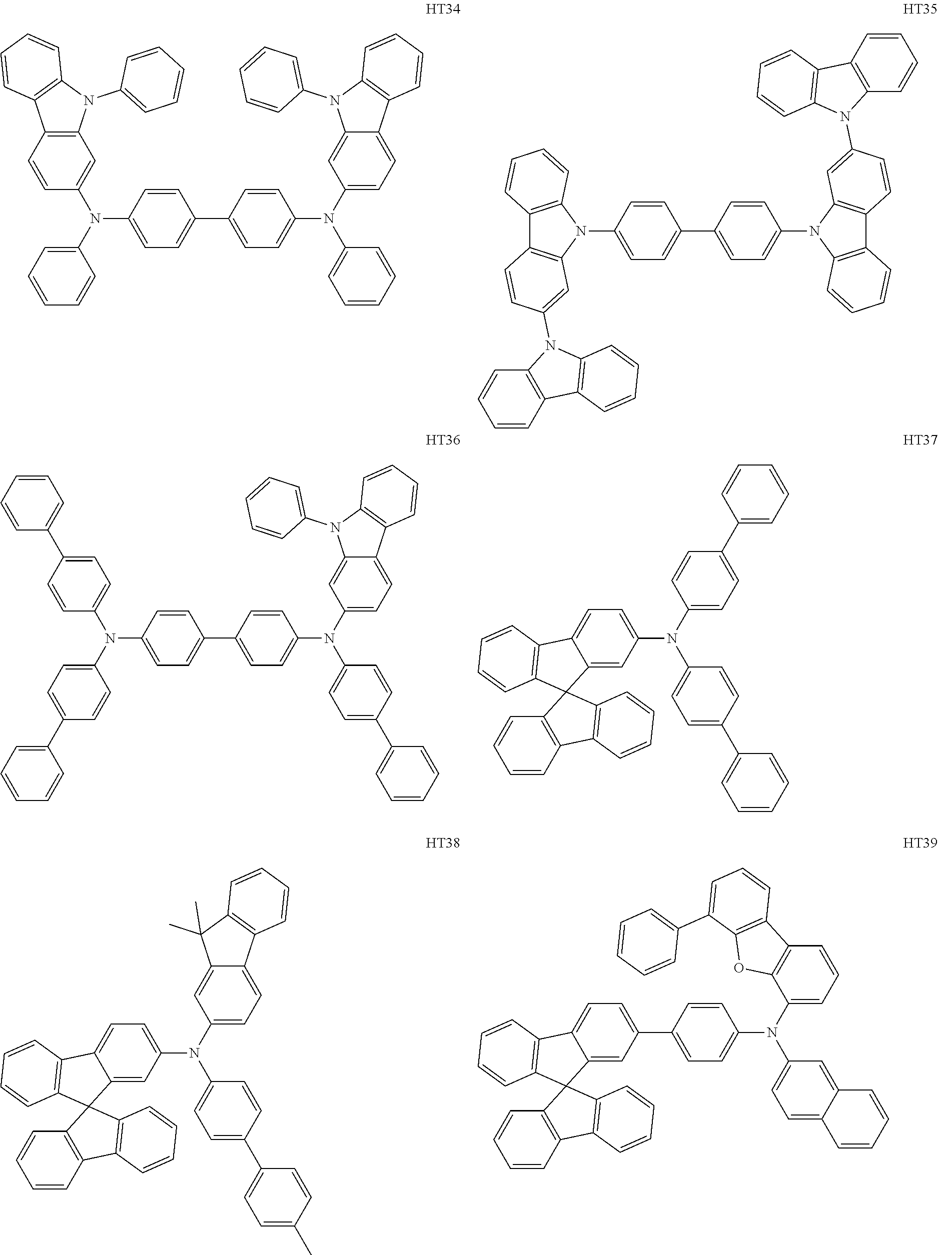

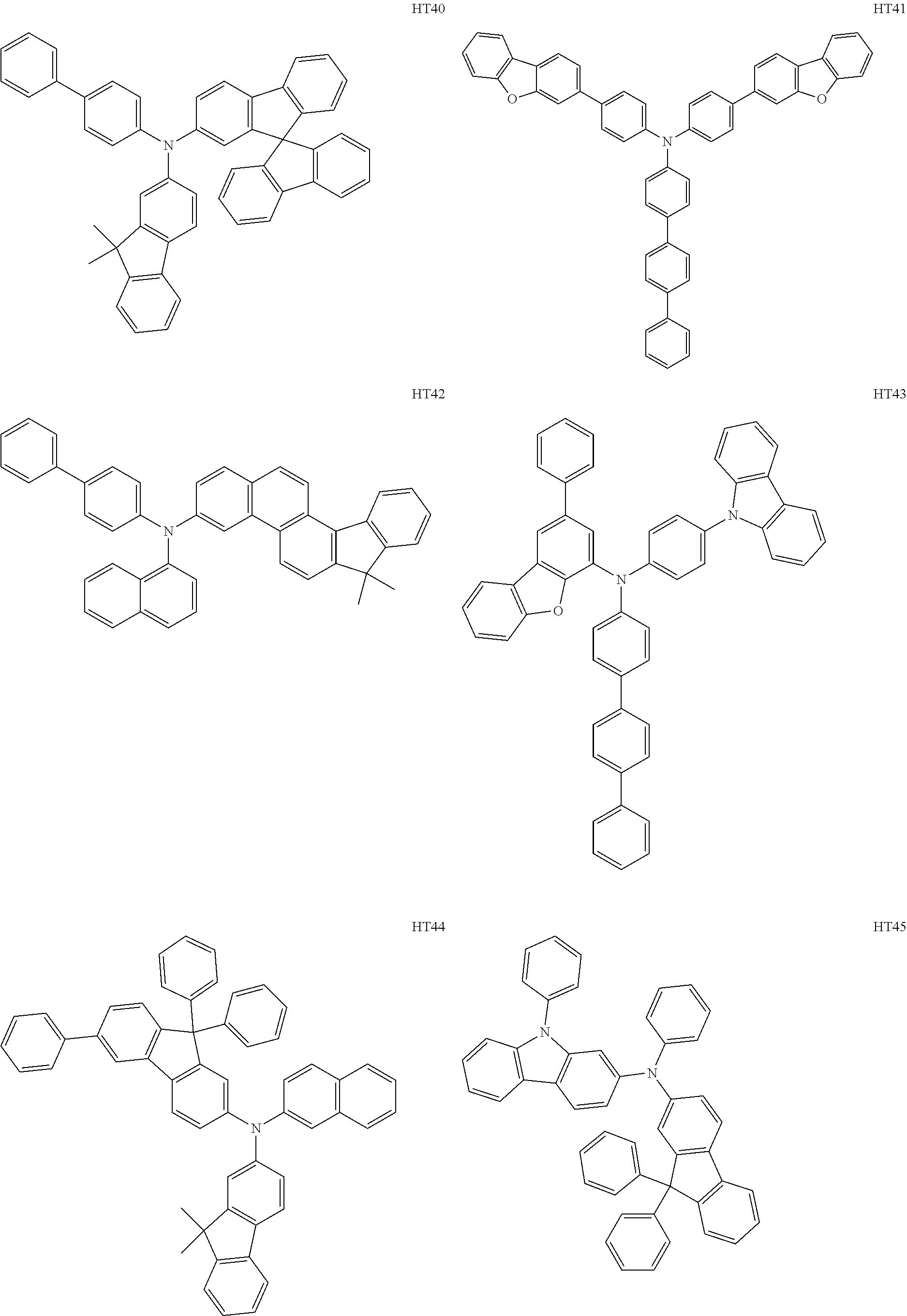

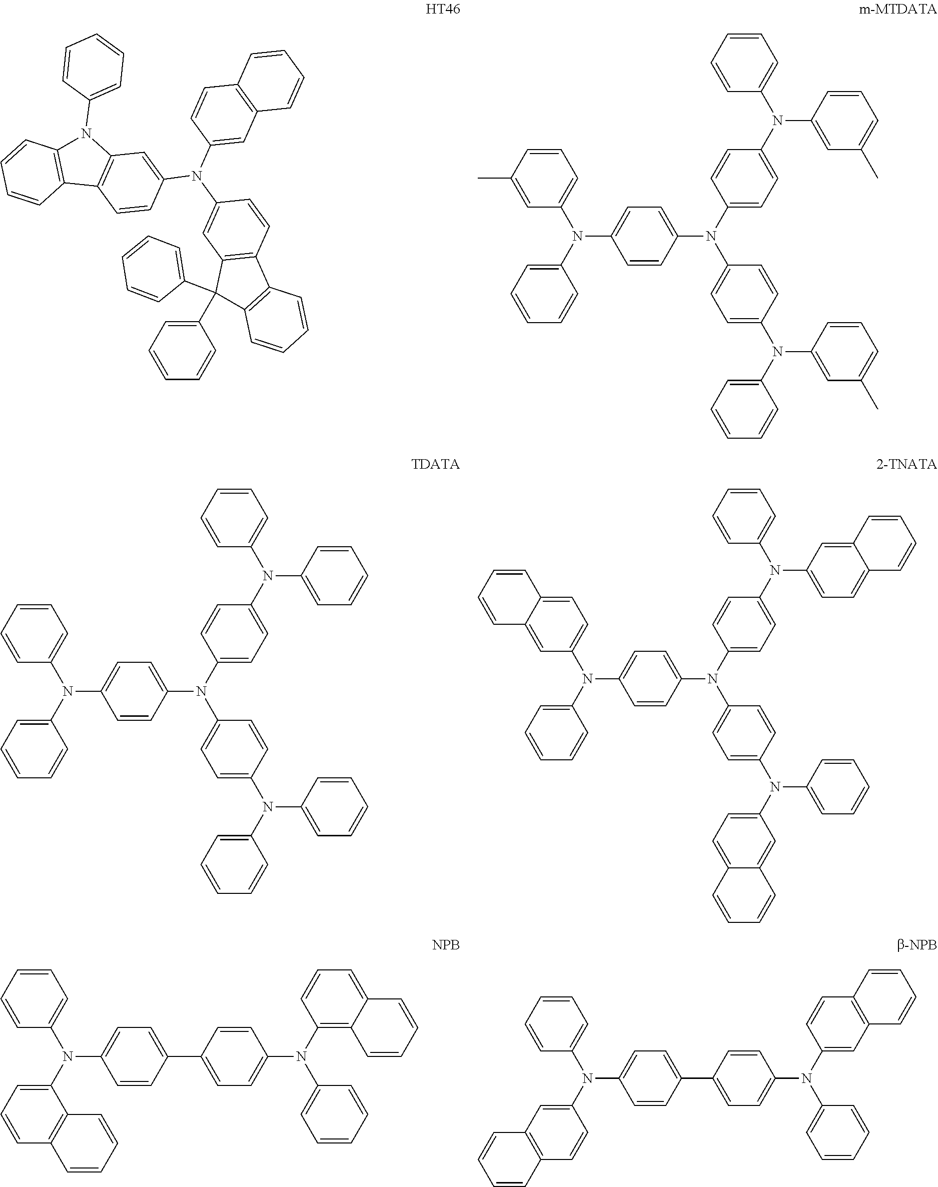

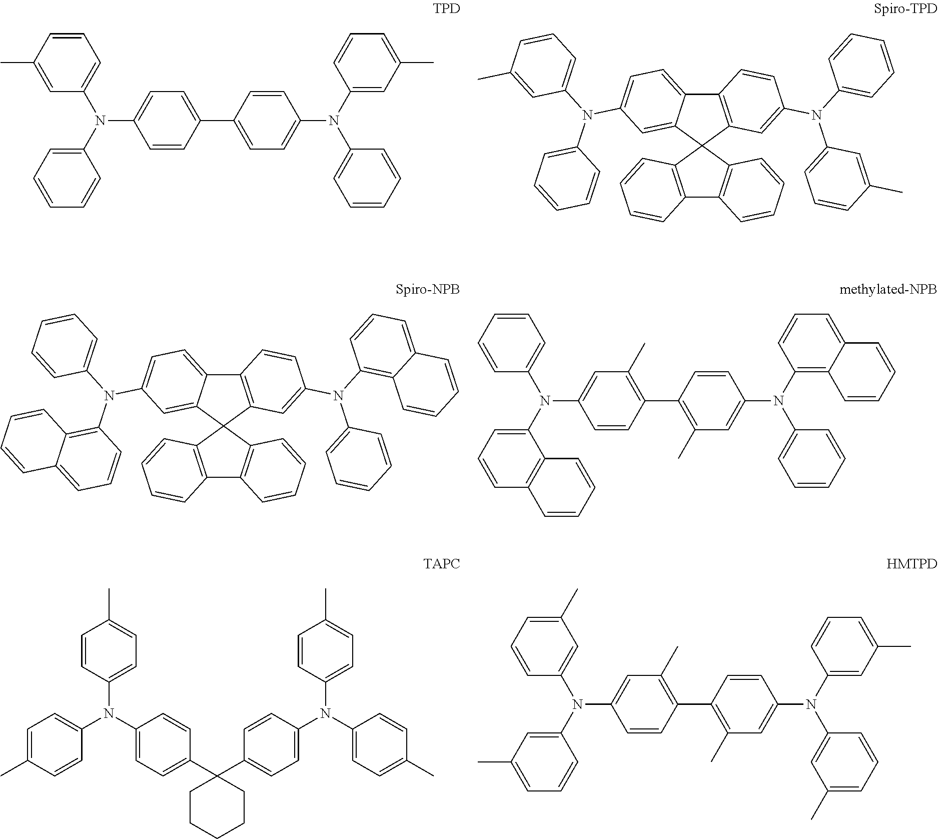

The thickness of the hole transport region may be about 50 Å to about 10,000 angstrom (Å), for example, about 100 Å to about 4,000 Å. When the hole transport region includes a hole injection layer, a hole transport layer, or any combination thereof, the thickness of the hole injection layer may be about 100 Å to about 9,000 Å, for example, about 100 Å to about 1,000 Å, and the thickness of the hole transport layer may be about 50 Å to about 2,000 Å, for example, about 100 Å to about 1,500 Å. When the thicknesses of the hole transport region, the hole injection layer, and the hole transport layer are within the ranges described, satisfactory hole transporting characteristics may be obtained without a substantial increase in driving voltage.

The emission auxiliary layer is a layer that increases light emission efficiency by compensating for an optical resonance distance according to a wavelength of light emitted from the emission layer 130. The electron blocking layer may be a layer that prevents or reduces electron leakage from the emission layer 130 to the hole transport region 120. Materials that may be included in the hole transport region may be included in the emission auxiliary layer and the electron blocking layer.

p-dopant

The hole transport region 120 may further include, in addition to the aforementioned materials, a charge-generation material for the improvement of conductive properties. The charge-generation material may be uniformly or non-uniformly dispersed in the hole transport region (for example, in the form of a single layer consisting of a charge-generation material).

The charge-generation material may be, for example, a p-dopant.

For example, the p-dopant may have a lowest unoccupied molecular orbital (LUMO) energy level of about −3.5 eV or less.

In one or more embodiments, the p-dopant may include a quinone derivative, a cyano group-containing compound, a compound including an element EL1 and an element EL2, or any combination thereof.

Examples of the quinone derivative may include TCNQ and F4-TCNQ.

Examples of the cyano group-containing compound may include HAT-CN and a compound represented by Formula 221.

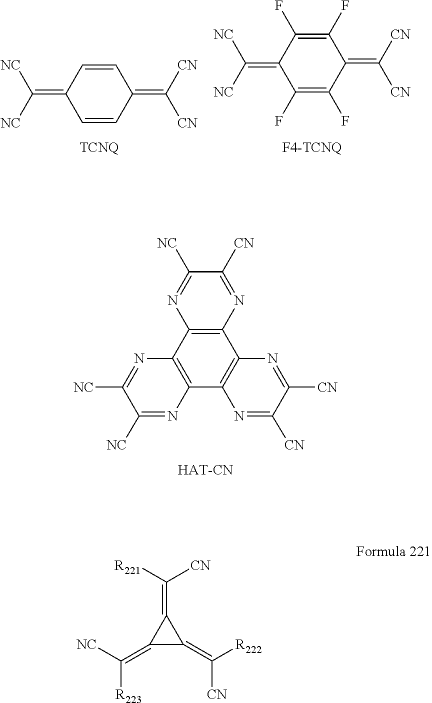

In Formula 221,
R₂₂₁ to R₂₂₃ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ, and
at least one of R₂₂₁ to R₂₂₃ may each independently be a C₃-C₆₀ carbocyclic group or a C₁-C₆₀ heterocyclic group, each substituted with: a cyano group; —F; —C₁; —Br; —I; a C₁-C₂₀ alkyl group substituted with a cyano group, —F, —Cl, —Br, —I, or any combination thereof; or any combination thereof.

In the compound including the element EL1 and the element EL2, the element EL1 may be a metal, a metalloid, or a combination thereof, and the element EL2 may be a non-metal, a metalloid, or a combination thereof.

Examples of the metal may include an alkali metal (for example, lithium (L₁), sodium (Na), potassium (K), rubidium (Rb), cesium (Cs), and/or the like); an alkaline earth metal (for example, beryllium (Be), magnesium (Mg), calcium (Ca), strontium (Sr), barium (Ba), and/or the like); a transition metal (for example, titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re), iron (Fe), ruthenium (Ru), osmium (Os), cobalt (Co), rhodium (Rh), iridium (Ir), nickel (Ni), palladium (Pd), platinum (Pt), copper (Cu), silver (Ag), gold (Au), and/or the like); a post-transition metal (for example, zinc (Zn), indium (In), tin (Sn), and/or the like); and a lanthanide metal (for example, lanthanum (La), cerium (Ce), praseodymium (Pr), neodymium (Nd), promethium (Pm), samarium (Sm), europium (Eu), gadolinium

Examples of the metalloid may include silicon (Si), antimony (Sb), and/or tellurium (Te).

Examples of the non-metal may include oxygen (O) and halogen (for example, F, Cl, Br, I, and/or the like).

Examples of the compound including the element EL1 and the element EL2 may include a metal oxide, a metal halide (for example, a metal fluoride, a metal chloride, a metal bromide, a metal iodide, and/or the like), a metalloid halide (for example, a metalloid fluoride, a metalloid chloride, a metalloid bromide, a metalloid iodide, and/or the like), a metal telluride, or any combination thereof.

Examples of the metal oxide may include a tungsten oxide (for example, WO, W₂O₃, WO₂, WO₃, W₂O₅, and/or the like), a vanadium oxide (for example, VO, V₂O₃, VO₂, V₂O₅, and/or the like), a molybdenum oxide (for example, MoO, Mo₂O₃, MoO₂, MoO₃, Mo₂O₅, and/or the like), a rhenium oxide (for example, ReO₃, and/or the like), and/or the like.

Examples of the metal halide may include an alkali metal halide, an alkaline earth metal halide, a transition metal halide, a post-transition metal halide, and a lanthanide metal halide.

Examples of the alkali metal halide may include LiF, NaF, KF, RbF, CsF, LiCl, NaCl, KCl, RbCl, CsCl, LiBr, NaBr, KBr, RbBr, CsBr, LiI, NaI, KI, RbI, and CsI.

Examples of the alkaline earth metal halide may include BeF₂, MgF₂, CaF₂, SrF₂, BaF₂, BeCl₂, MgCl₂, CaCl₂), SrCl₂, BaCl₂, BeBr₂, MgBr₂, CaBr₂, SrBr₂, BaBr₂, BeI₂, MgI₂, CaI₂, SrI₂, and BaI₂.

Examples of the transition metal halide may include a titanium halide (for example, TiF₄, TiCl₄, TiBr₄, TiI₄, and/or the like), a zirconium halide (for example, ZrF₄, ZrCl₄, ZrBr₄, ZrI₄, and/or the like), a hafnium halide (for example, HfF₄, HfCl₄, HfBr₄, HfI₄, and/or the like), a vanadium halide (for example, VF₃, VCl₃, VBr₃, VI₃, and/or the like), a niobium halide (for example, NbF₃, NbCl₃, NbBr₃, NbI₃, and/or the like), a tantalum halide (for example, TaF₃, TaCl₃, TaBr₃, TaI₃, and/or the like), a chromium halide (for example, CrF₃, CrCl₃, CrBr₃, CrI₃, and/or the like), a molybdenum halide (for example, MoF₃, MoClK, MoBr₃, MoI₃, and/or the like), a tungsten halide (for example, WF₃, WCl₃, WBr₃, WI₃, and/or the like), a manganese halide (for example, MnF₂, MnCl₂, MnBr₂, MnI₂, and/or the like), a technetium halide (for example, TcF₂, TcCl₂, TcBr₂, TcI₂, and/or the like), a rhenium halide (for example, ReF₂, ReCl₂, ReBr₂, ReI₂, and/or the like), an iron halide (for example, FeF₂, FeCl₂, FeBr₂, FeI₂, and/or the like), a ruthenium halide (for example, RuF₂, RuCl₂, RuBr₂, RuI₂, and/or the like), an osmium halide (for example, OsF₂, OsCl₂, OsBr₂, OsI₂, and/or the like), a cobalt halide (for example, CoF₂, COCl₂, CoBr₂, CoI₂, and/or the like), a rhodium halide (for example, RhF₂, RhCl₂, RhBr₂, RhI₂, and/or the like), an iridium halide (for example, IrF₂, IrCl₂, IrBr₂, IrI₂, and/or the like), a nickel halide (for example, NiF₂, NiCl₂, NiBr₂, NiI₂, and/or the like), a palladium halide (for example, PdF₂, PdCl₂, PdBr₂, PdI₂, and/or the like), a platinum halide (for example, PtF₂, PtCl₂, PtBr₂, PtI₂, and/or the like), a copper halide (for example, CuF, CuCl, CuBr, CuI, and/or the like), a silver halide (for example, AgF, AgCl, AgBr, AgI, and/or the like), and a gold halide (for example, AuF, AuCl, AuBr, AuI, and/or the like).

Examples of the post-transition metal halide may include a zinc halide (for example, ZnF₂, ZnCl₂, ZnBr₂, ZnI₂, and/or the like), an indium halide (for example, InI₃, and/or the like), a tin halide (for example, SnI₂, and/or the like), and/or the like.

Examples of the lanthanide metal halide may include YbF, YbF₂, YbF₃, SmF₃, YbCl, YbCl₂, YbCl₃ SmCl₃, YbBr, YbBr₂, YbBr₃, SmBr₃, YbI, YbI₂, YbI₃, SmI₃, and/or the like.

Examples of the metalloid halide may include an antimony halide (for example, SbCl₅, and/or the like).

Examples of the metal telluride may include an alkali metal telluride (for example, Li₂Te, Na₂Te, K₂Te, Rb₂Te, Cs₂Te, and/or the like), an alkaline earth metal telluride (for example, BeTe, MgTe, CaTe, SrTe, BaTe, and/or the like), a transition metal telluride (for example, TiTe₂, ZrTe₂, HfTe₂, V₂Te₃, Nb₂Te₃, Ta₂Te₃, Cr₂Te₃, Mo₂Te₃, W₂Te₃, MnTe, TcTe, ReTe, FeTe, RuTe, OsTe, CoTe, RhTe, IrTe, NiTe, PdTe, PtTe, Cu₂Te, CuTe, Ag₂Te, AgTe, Au₂Te, and/or the like), a post-transition metal telluride (for example, ZnTe, and/or the like), and a lanthanide metal telluride (for example, LaTe, CeTe, PrTe, NdTe, PmTe, EuTe, GdTe, TbTe, DyTe, HoTe, ErTe, TmTe, YbTe, LuTe, and/or the like).

Emission layer 130

When the light-emitting device 10 is a full-color light-emitting device, the emission layer may be patterned into a red emission layer, a green emission layer, and/or a blue emission layer, according to a sub-pixel. In one or more embodiments, the emission layer may have a stacked structure of two or more layers of a red emission layer, a green emission layer, and a blue emission layer, in which the two or more layers contact each other or are separated from each other, to emit white light. In one or more embodiments, the emission layer may include two or more materials of a red light-emitting material, a green light-emitting material, and a blue light-emitting material, in which the two or more materials are mixed with each other in a single layer, to emit white light.

The emission layer may include a host and a dopant. The dopant may include a phosphorescent dopant, a fluorescent dopant, or any combination thereof.

The amount of the dopant in the emission layer may be from about 0.01 part by weight to about 15 parts by weight based on 100 parts by weight of the host.

In one or more embodiments, the emission layer may include a quantum dot.

In some embodiments, the emission layer may include a delayed fluorescence material. The delayed fluorescence material may act as a host or a dopant in the emission layer.

The thickness of the emission layer may be about 100 Å to about 1,000 Å, for example, about 200 Å to about 600 Å. When the thickness of the emission layer is within the range described, excellent or suitable luminescence characteristics may be obtained without a substantial increase in driving voltage.

### Host

The host may include a compound represented by Formula 301:

[Ar₃₀₁]_{xb11}-[(L₃₀₁)_{xb1}-R₃₀₁]_{xb21} Formula 301

In Formula 301,
Ar₃₀₁ and L₃₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xb11 may be 1, 2, or 3,
xb1 may be an integer selected from 0 to 5,
R₃₀₁ may be hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₃₀₁)(Q₃₀₂)(Q₃₀₃), —N(Q₃₀₁)(Q₃₀₂), —B(Q₃₀₁)(Q₃₀₂), —C(═O)(Q₃₀₁), —S(═O)₂(Q₃₀₁), or —P(═O)(Q₃₀₁)(Q₃₀₂),
xb21 may be an integer selected from 1 to 5, and
Q₃₀₁ to 0303 are each as described in connection with Q₁ herein.

In one or more embodiments, when xb11 in Formula 301 is 2 or more, two or more of Ar₃₀₁ may be linked together via a single bond.

In one or more embodiments, the host may include a compound represented by Formula 301-1, a compound represented by Formula 301-2, or any combination thereof:

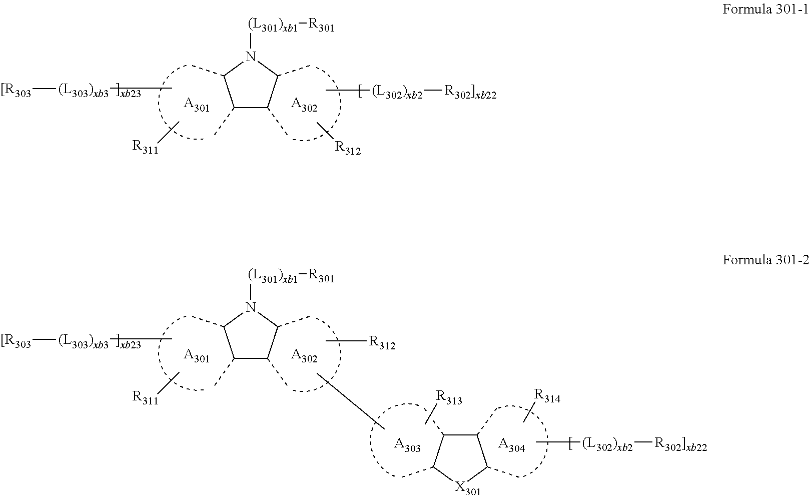

wherein, in Formula 301-1 and 301-2,
ring A₃₀₁ to ring A₃₀₄ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₃₀₁ may be O, S, N[(L₃₀₄)_{xb4}-R₃₀₄], C(R₃₀₄)(R₃₀₅), or Si(R₃₀₄)(R₃₀₅),
xb22 and xb23 may each independently be 0, 1, or 2,
L₃₀₁, xb1, and R₃₀₁ are each as described herein,
L₃₀₂ to L₃₀₄ may each independently be as described in connection with L₃₀₁,
xb2 to xb4 may each independently be as described in connection with xb1, and
R₃₀₂ to R₃₀₅ and R₃₁₁ to R₃₁₄ are each as described in connection with R₃₀₁.

In one or more embodiments, the host may include an alkali earth metal complex, a post-transition metal complex, or any combination thereof. In one or more embodiments, the host may include a Be complex (for example, Compound H55), an Mg complex, a Zn complex, or any combination thereof.

In one or more embodiments, the host may include: at least one of Compounds H1 to H128; 9,10-di(2-naphthyl)anthracene (ADN); 2-methyl-9,10-bis(naphthalen-2-yl)anthracene (MADN); 9,10-di-(2-naphthyl)-2-t-butyl-anthracene (TBADN); 4,4′-bis(N-carbazolyl)-1,1′-biphenyl (CBP); 1,3-di(carbazol-9-yl)benzene (mCP); 1,3,5-tri(carbazol-9-yl)benzene (TCP); or any combination thereof:

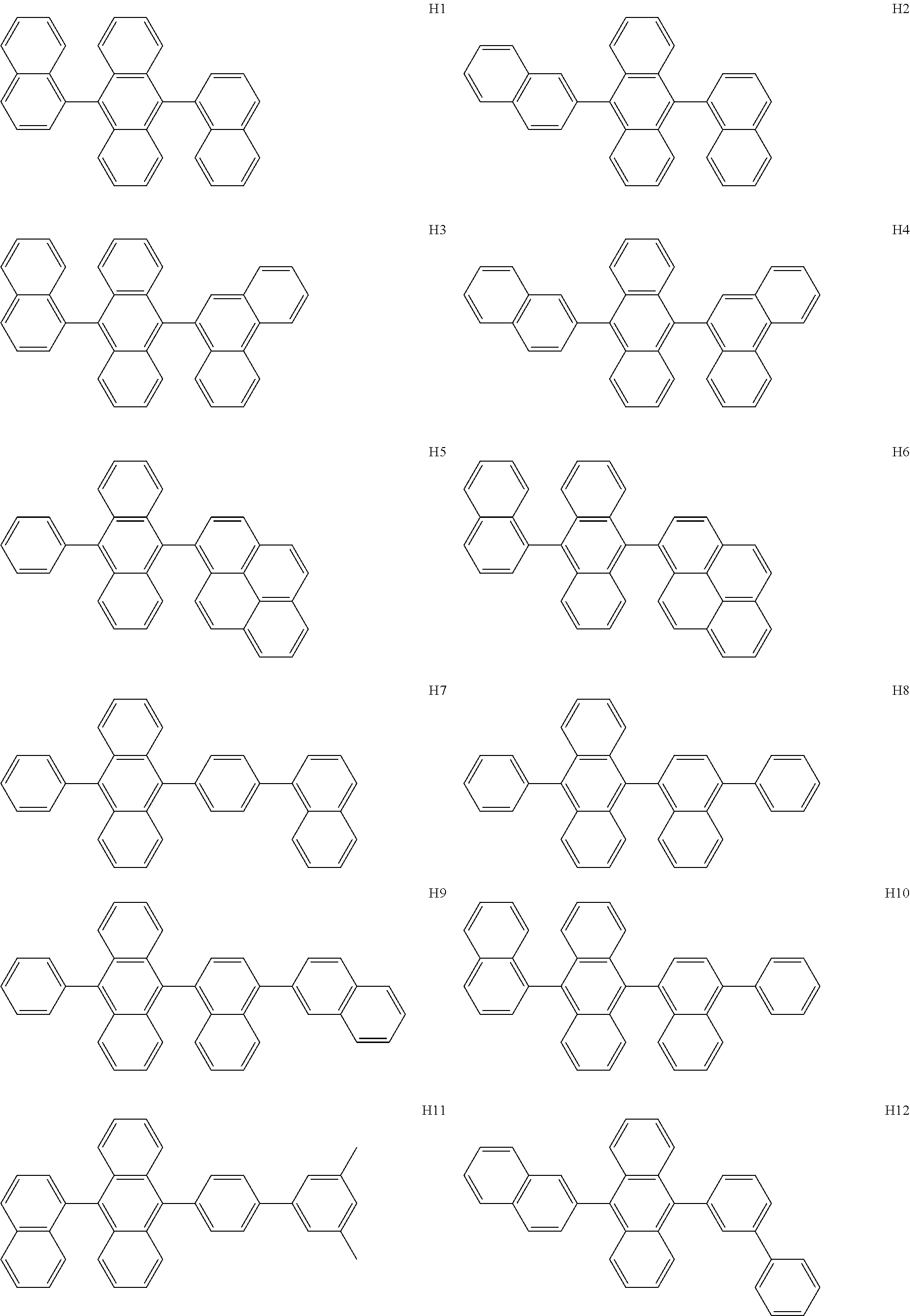

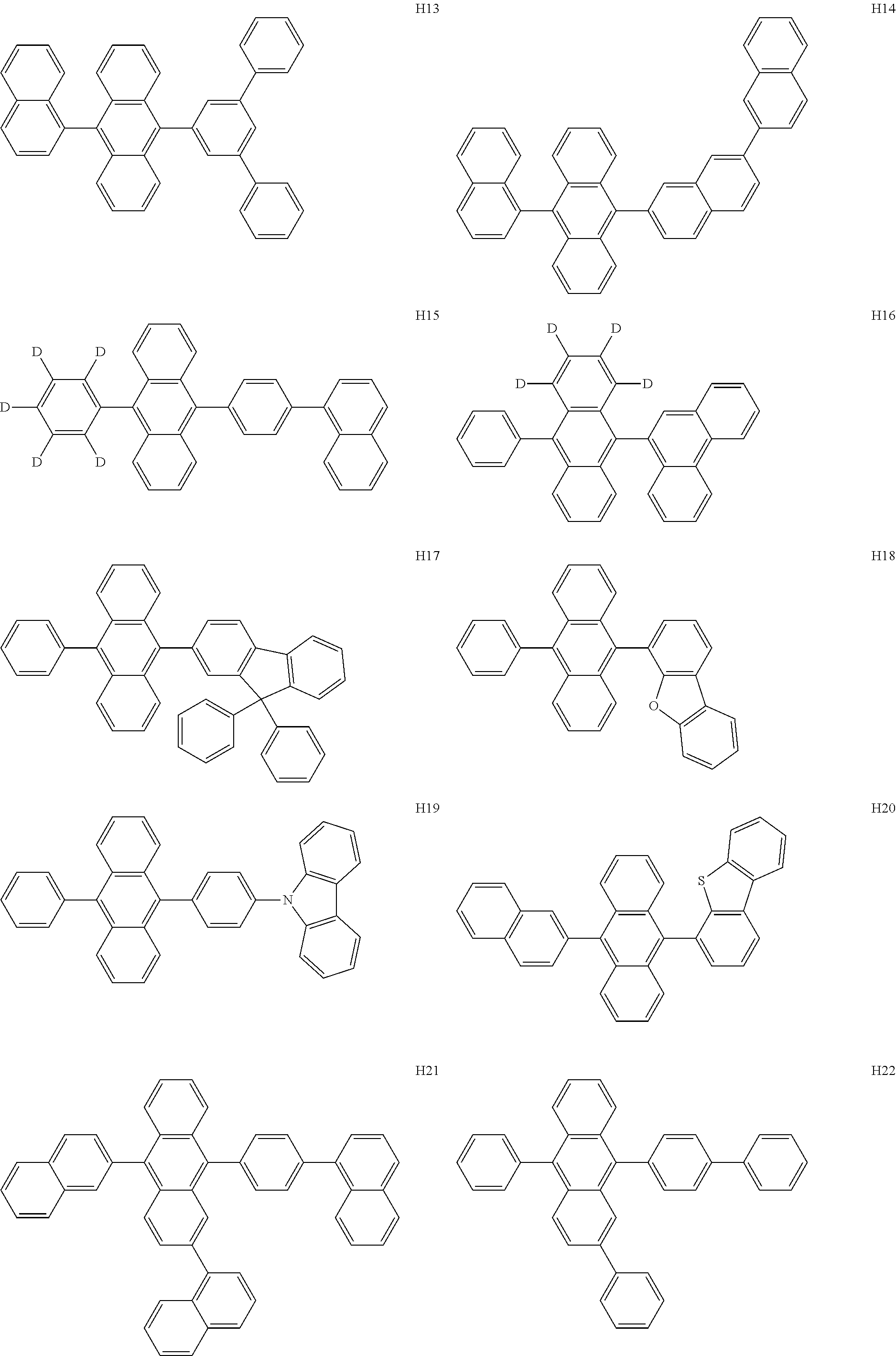

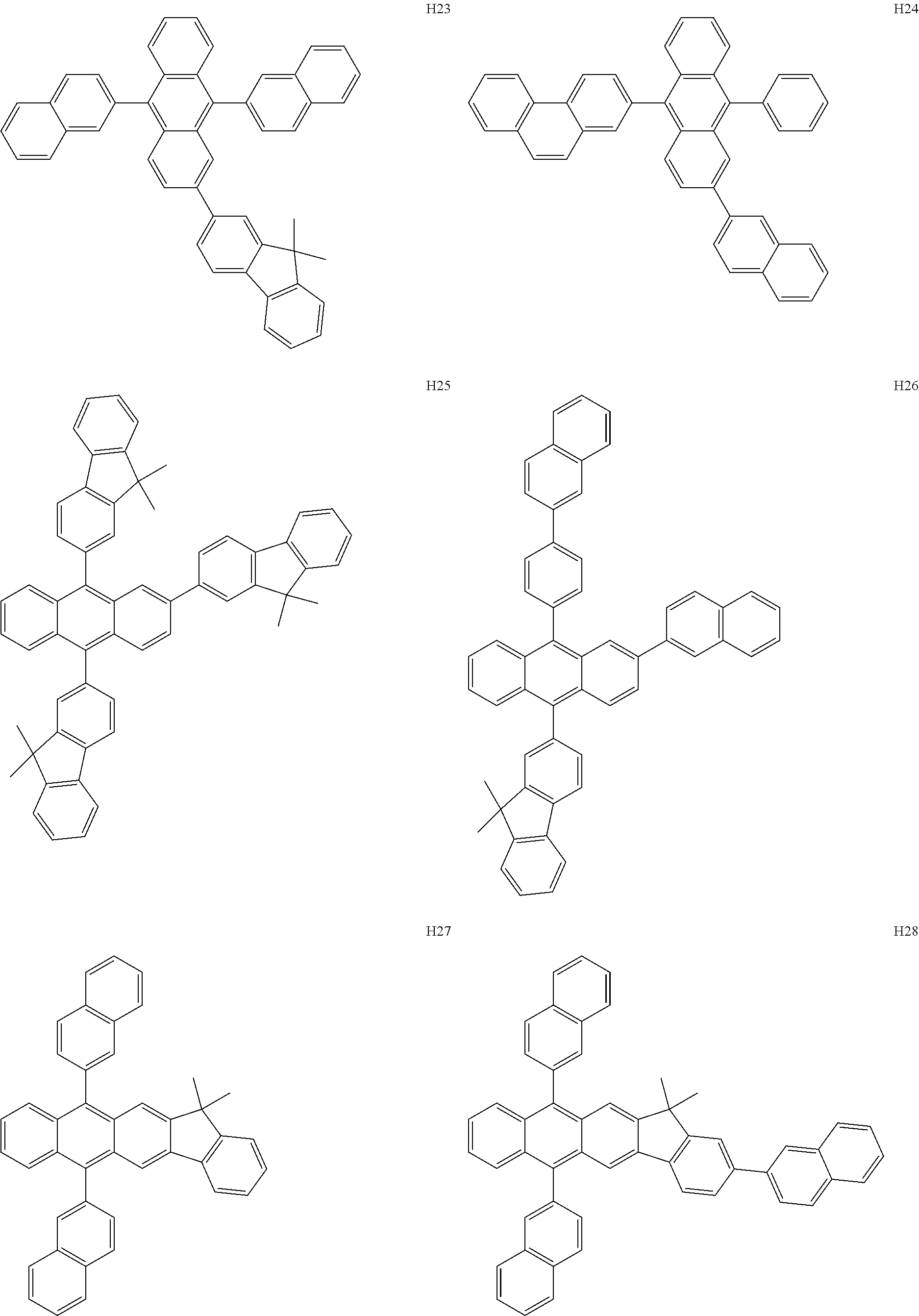

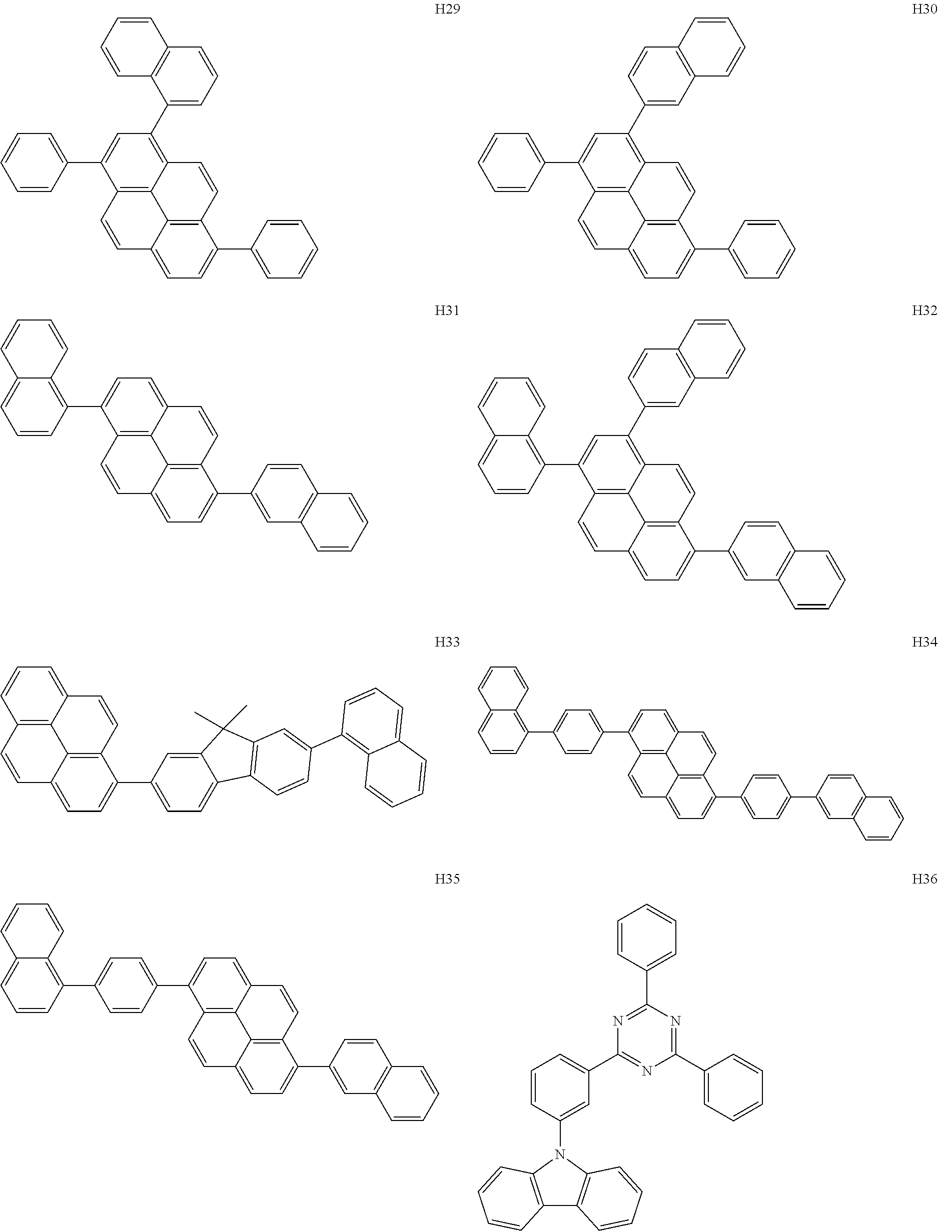

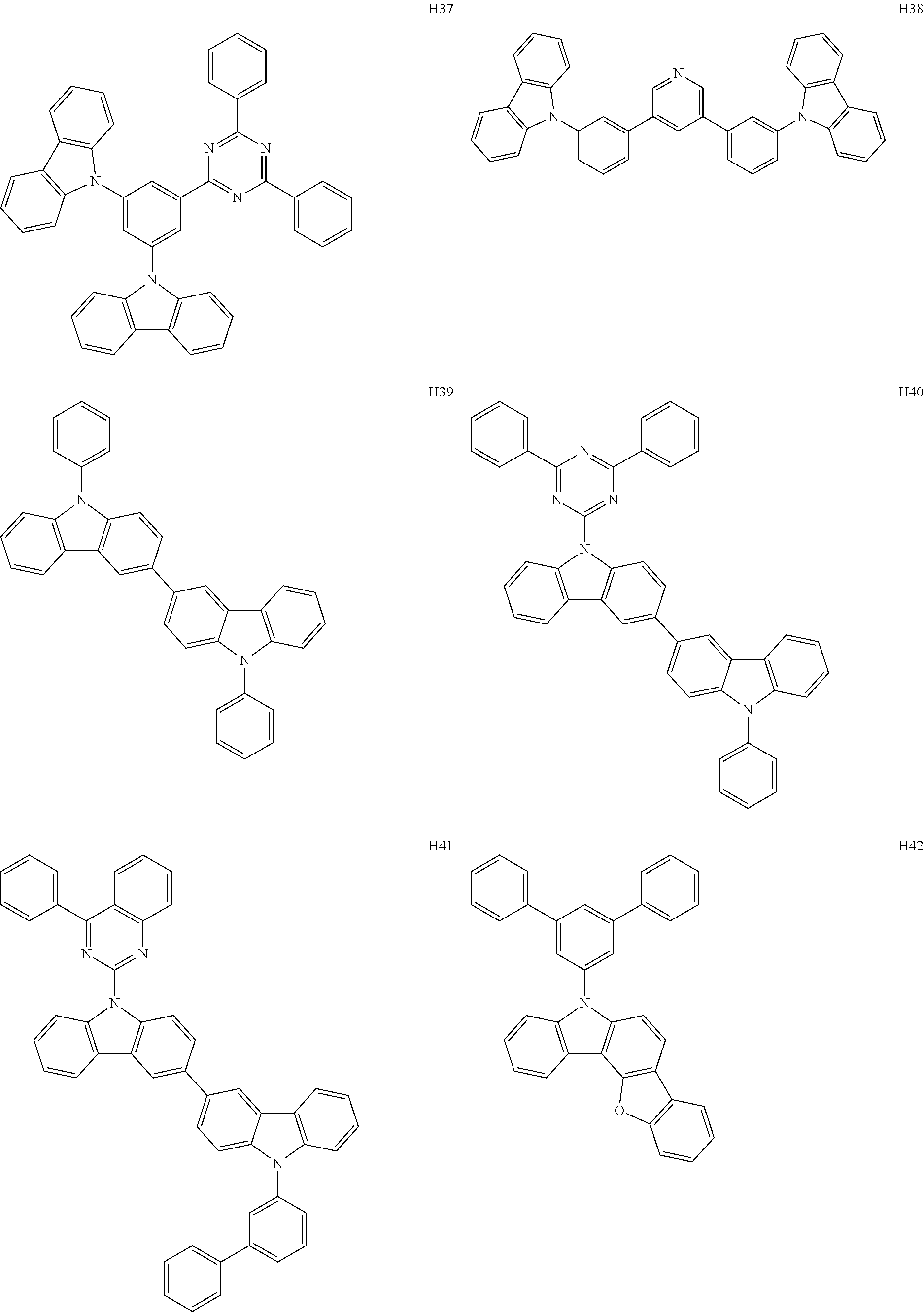 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-n2c3ccccc3c3cc(-c4ccc5c(c4)c4ccccc4n5-c4ccccc4)ccc32)cc1 ; c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2cccc(-n3c4ccccc4c4cc(-c5ccc6c(c5)c5ccccc5n6-c5nc(-c6ccccc6)c6ccccc6n5)ccc43)c2)cc1 ; c1ccc(-c2cc(-c3ccccc3)cc(-n3c4ccccc4c4c5oc6ccccc6c5ccc43)c2)cc1]

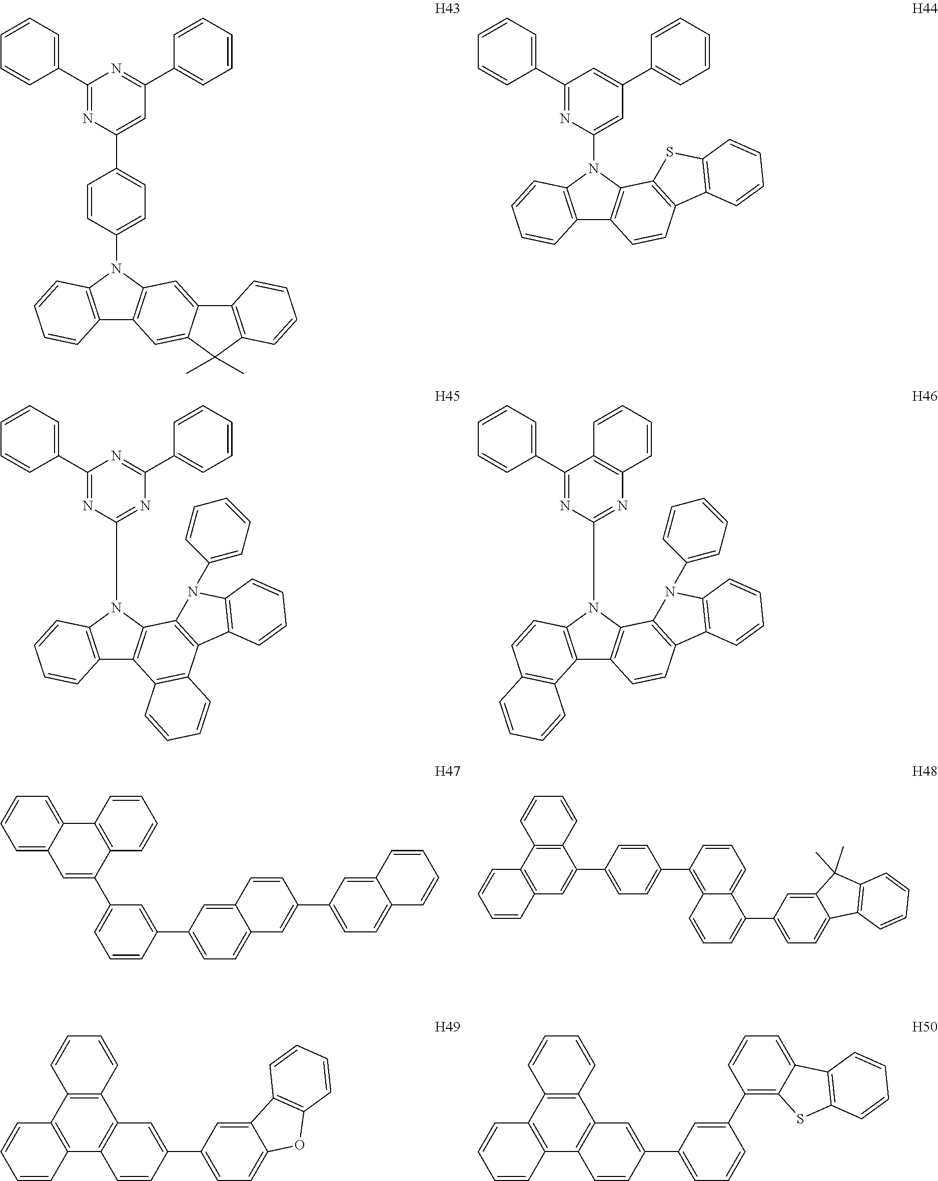

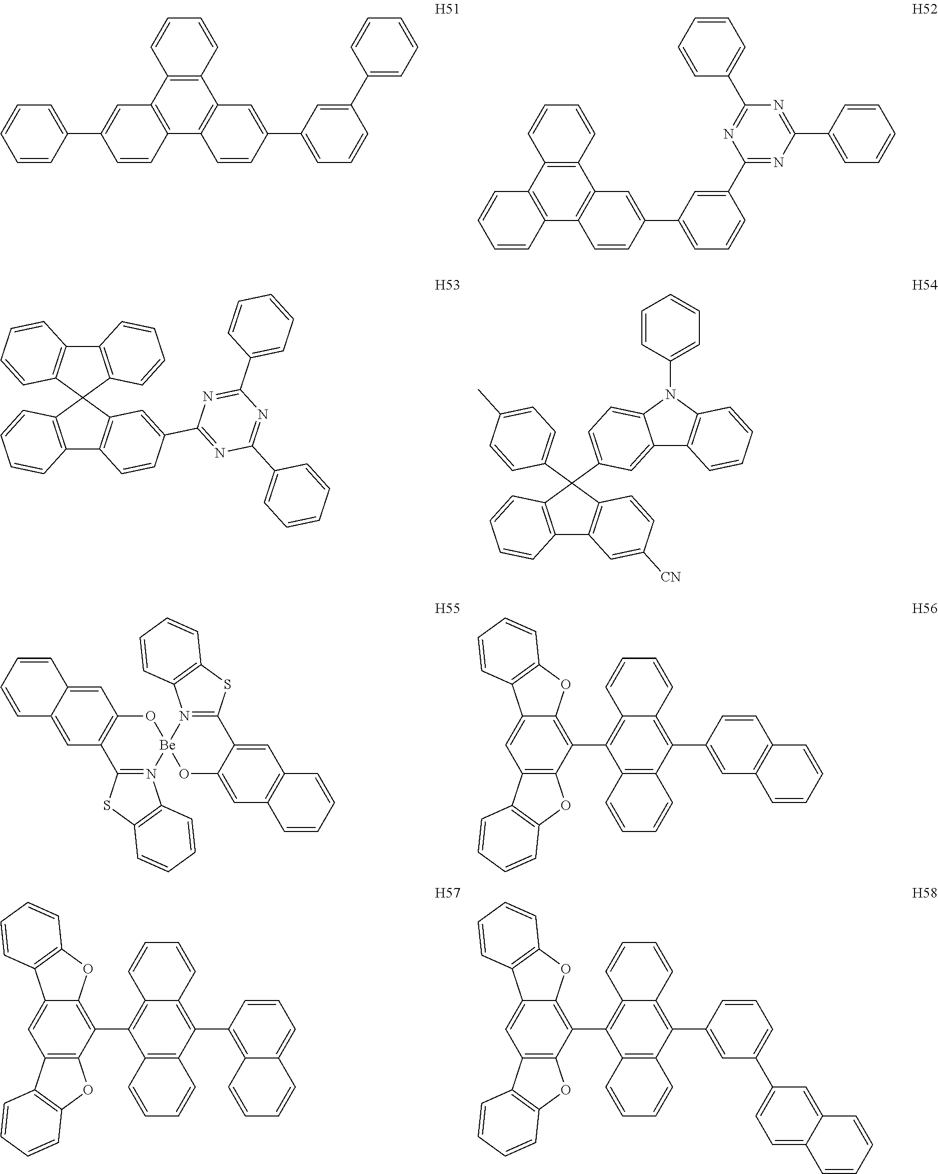

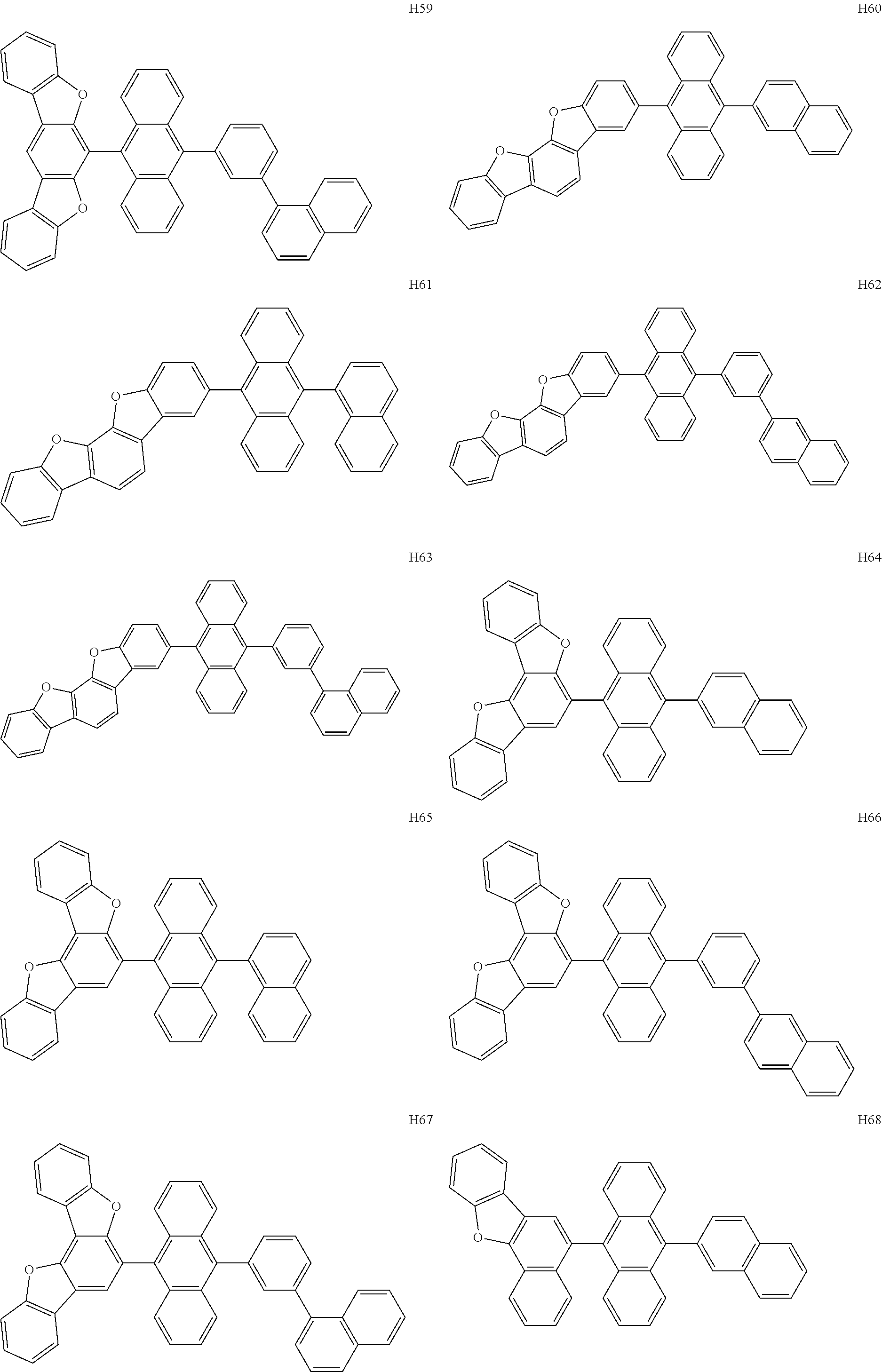

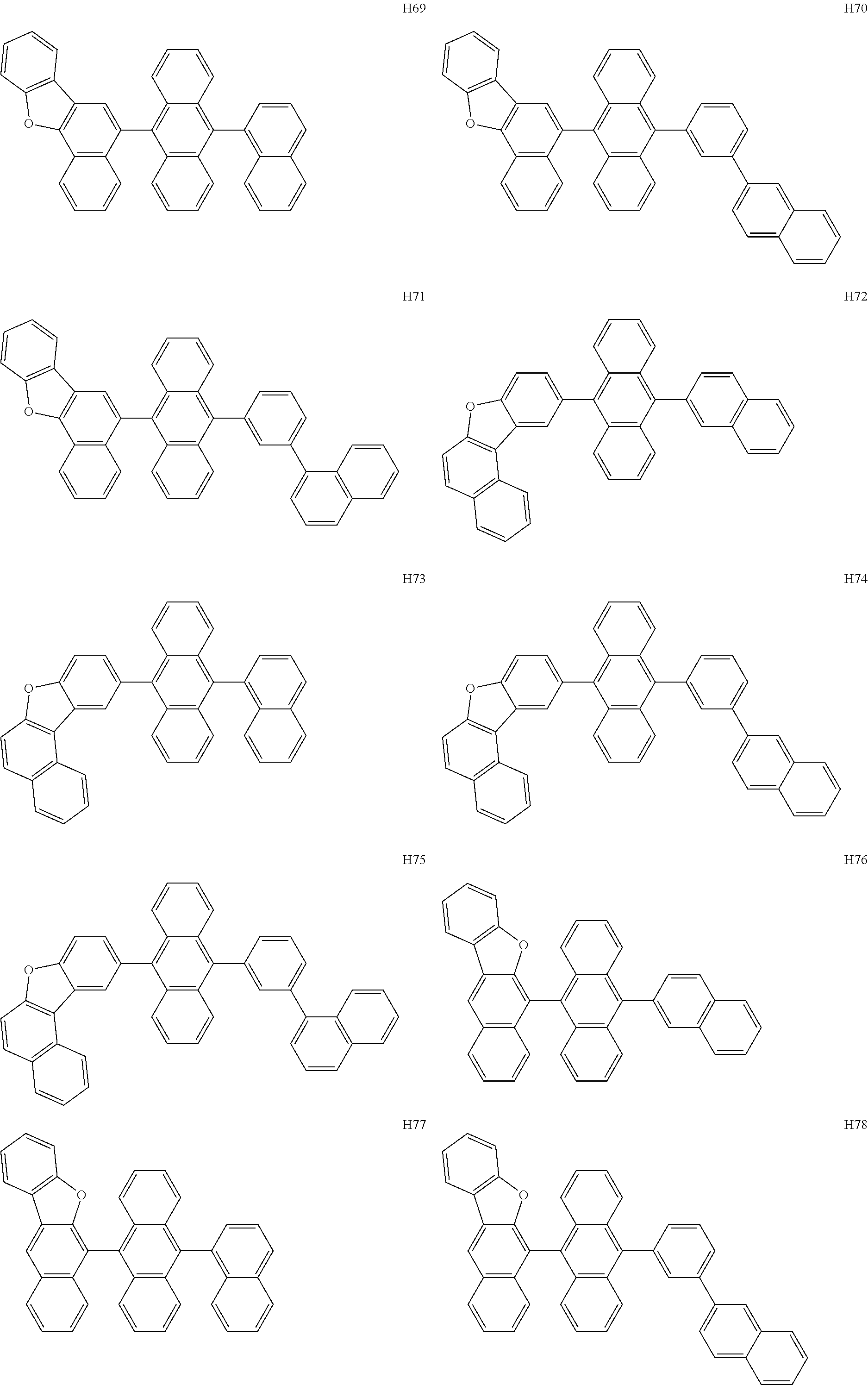

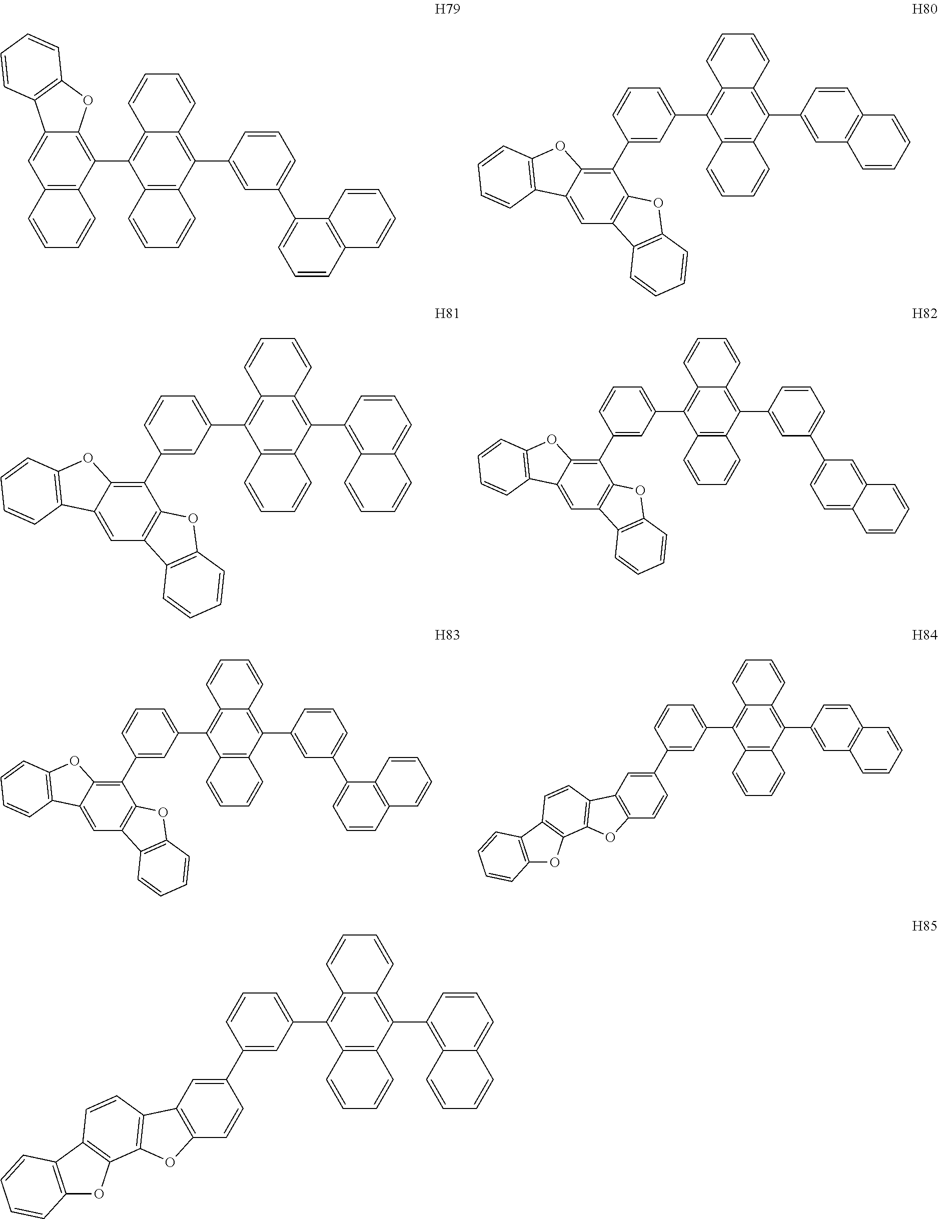

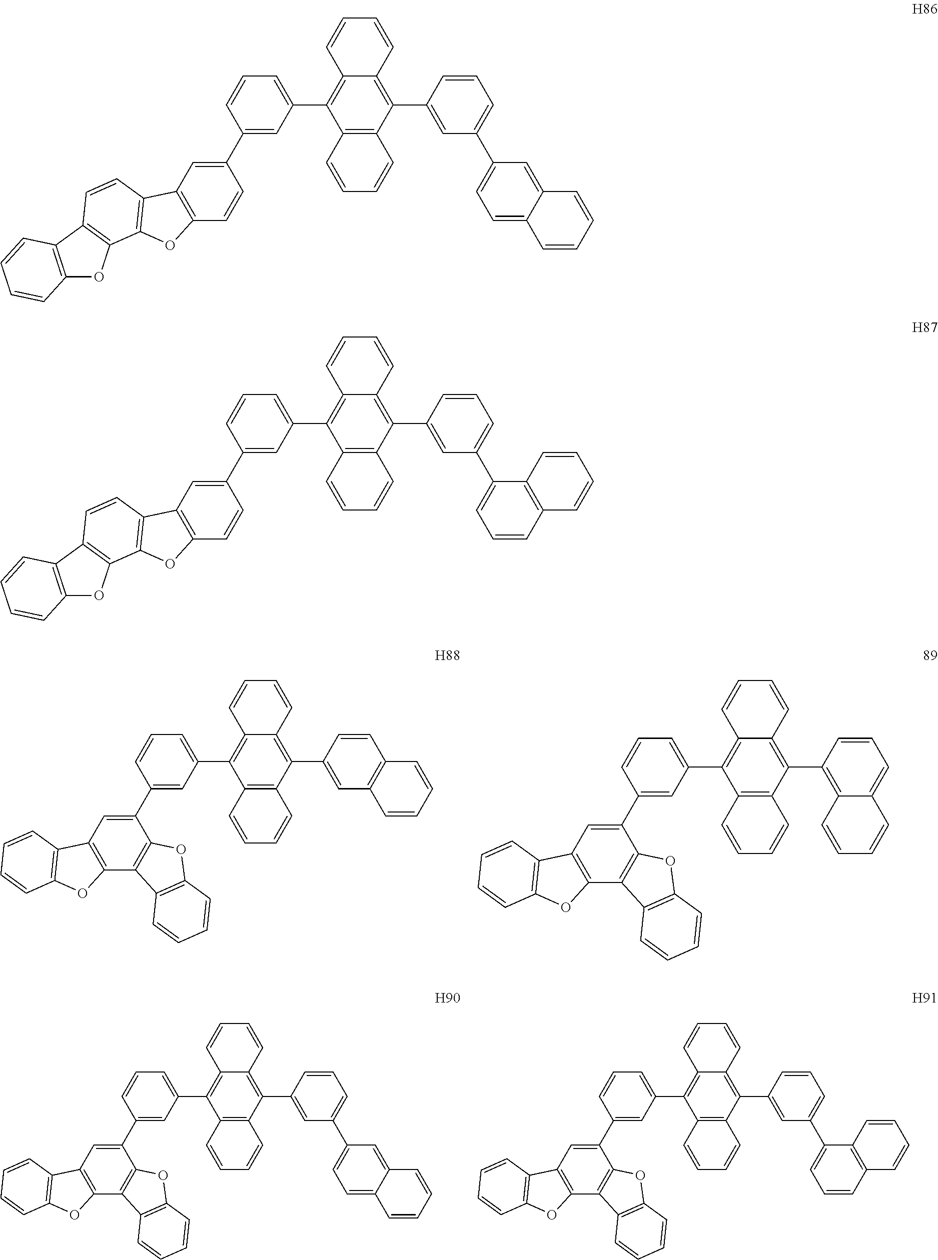

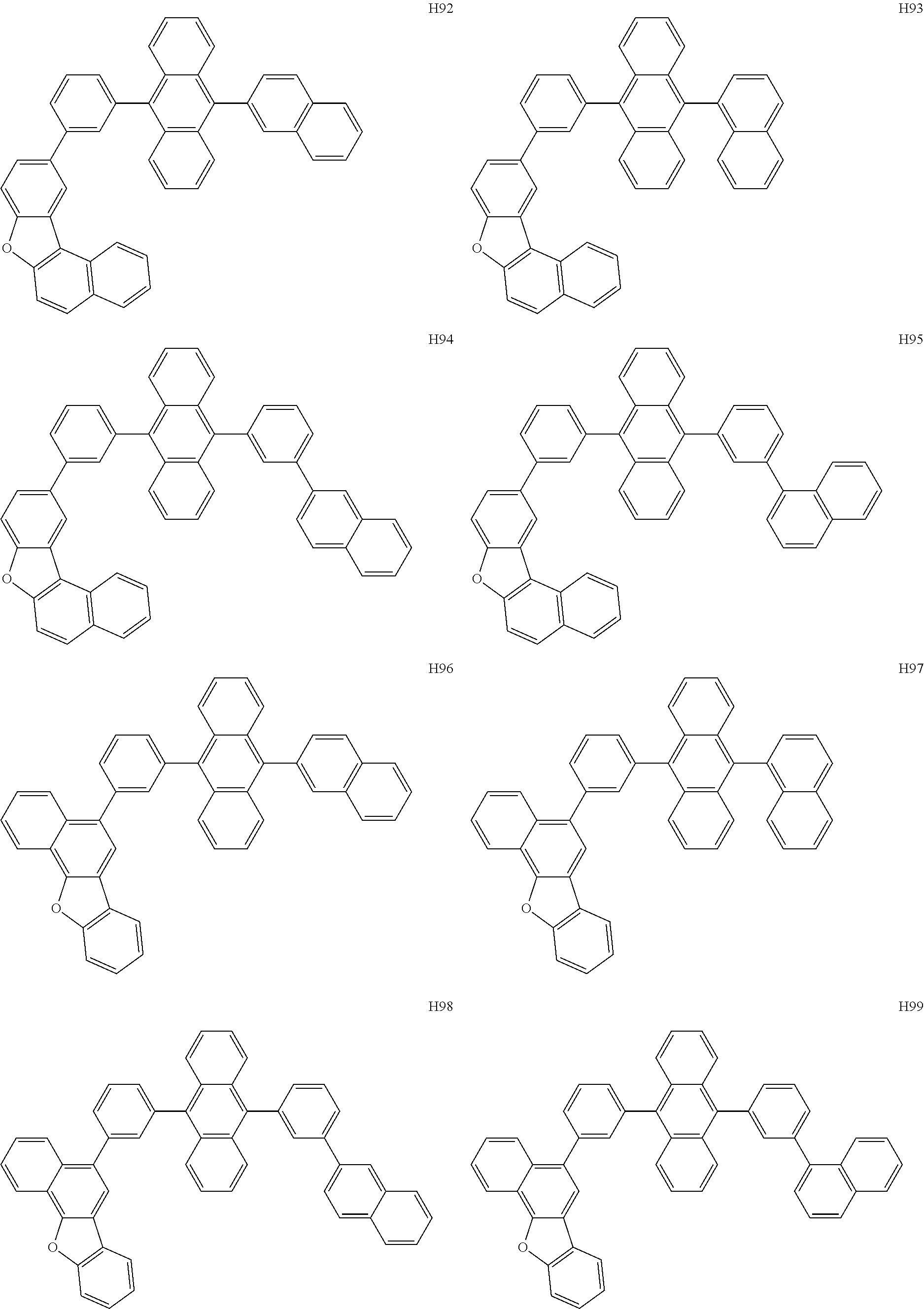

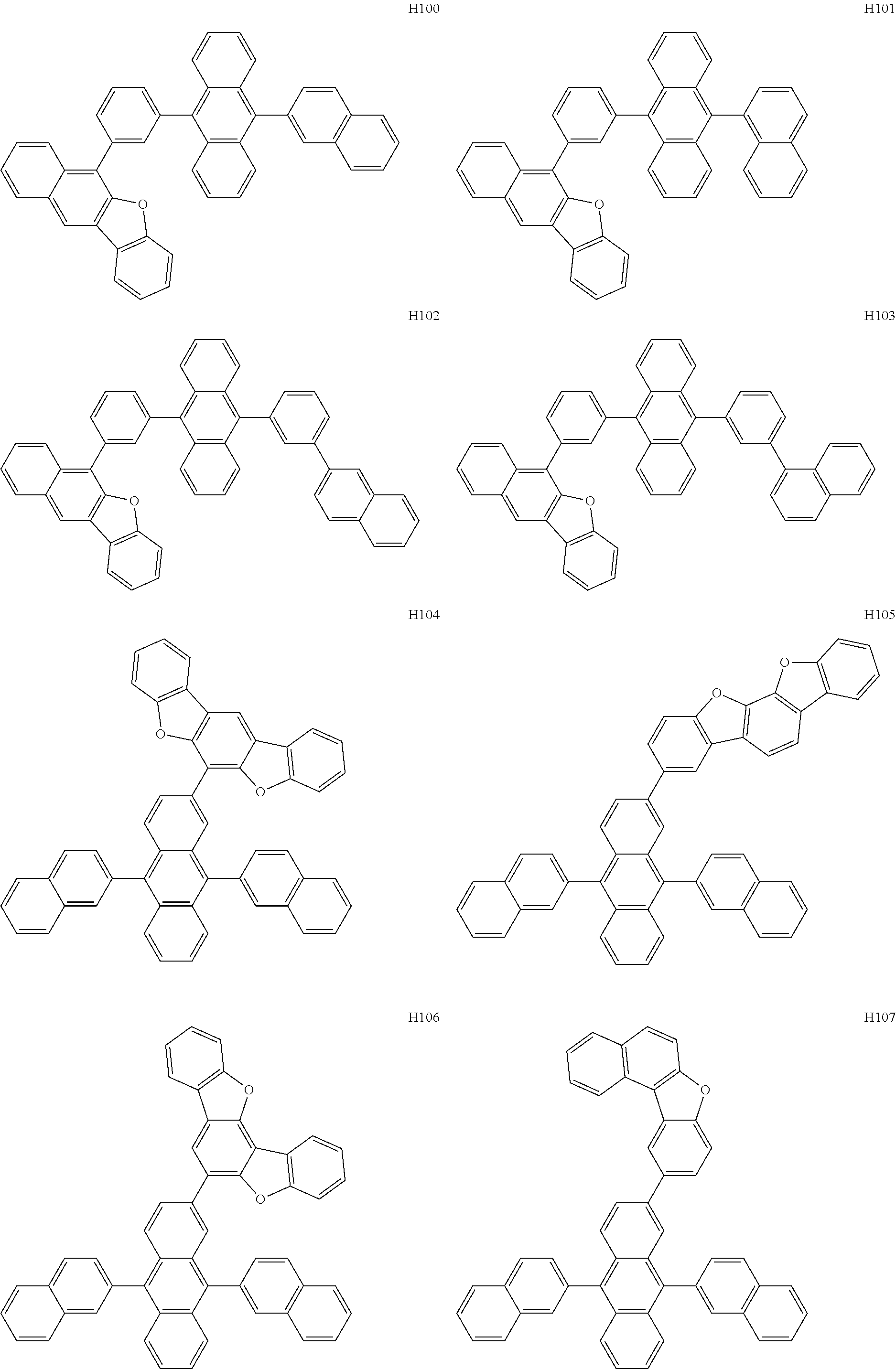

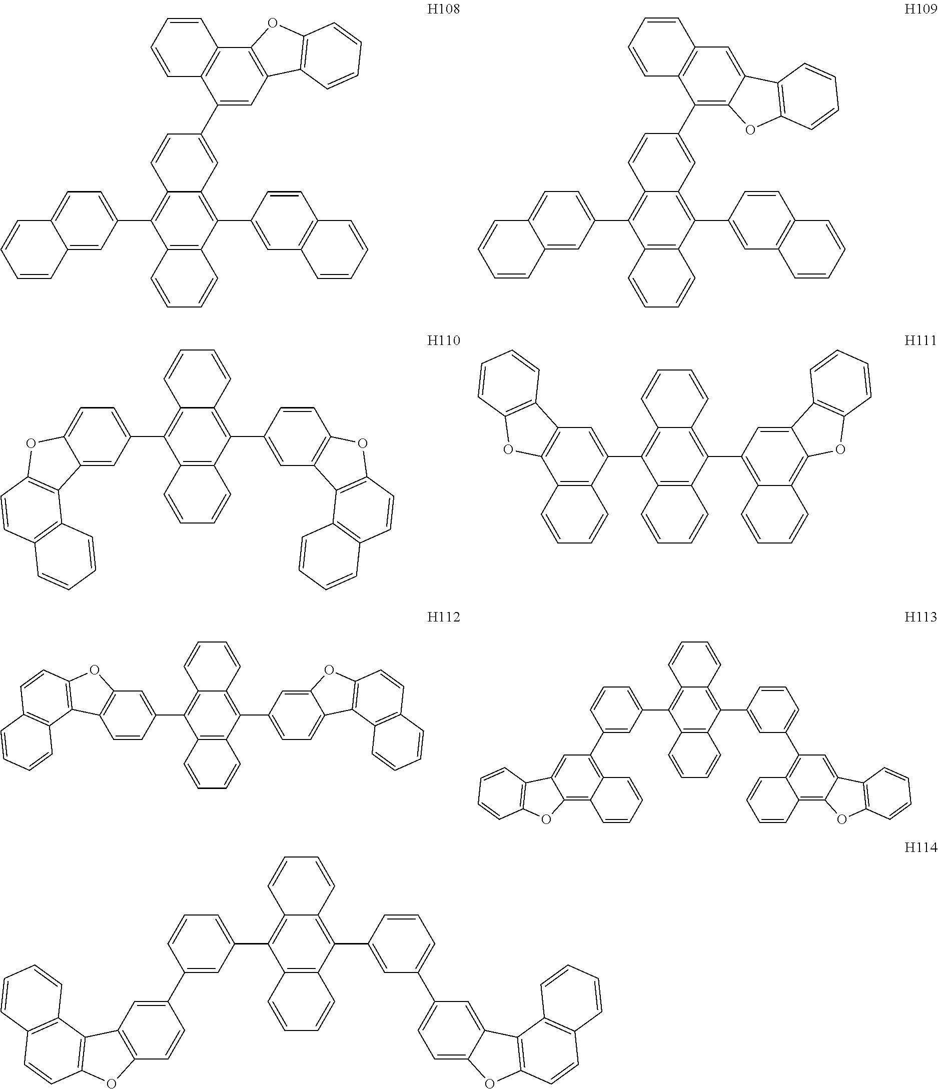

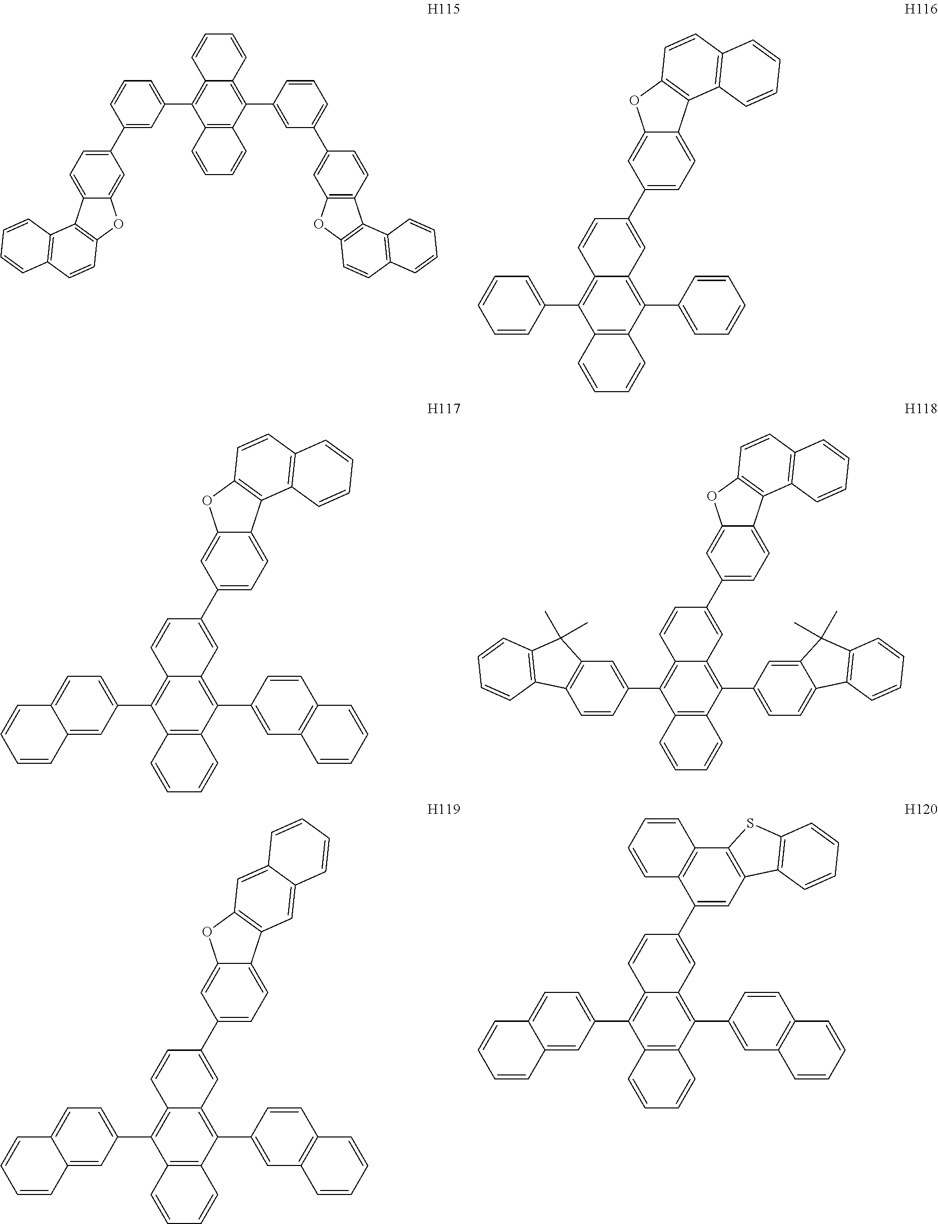

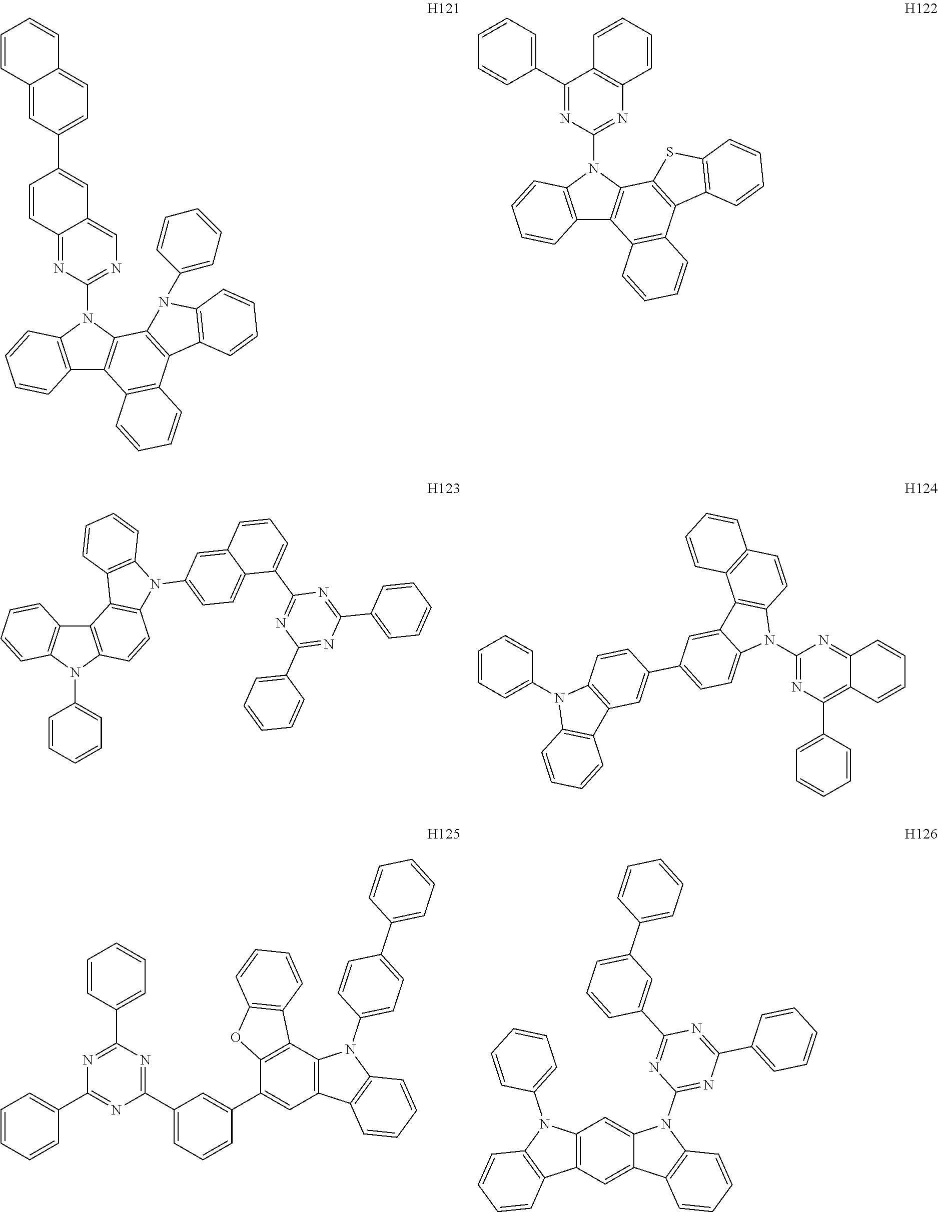

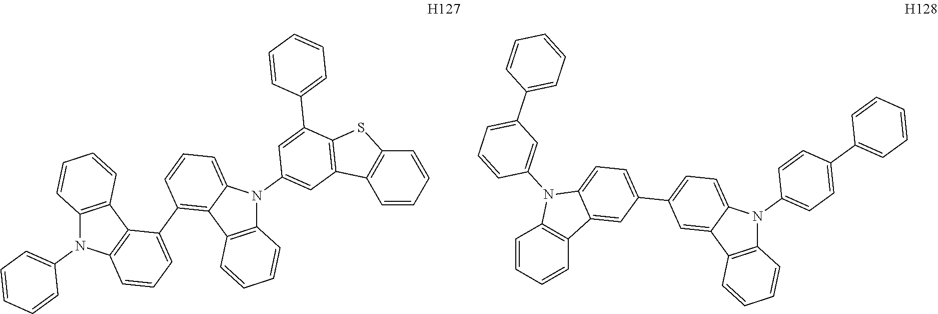 [SMILES: c1ccc(-c2cc(-n3c4ccccc4c4c(-c5cccc6c5c5ccccc5n6-c5ccccc5)cccc43)cc3c2sc2ccccc23)cc1 ; c1ccc(-c2ccc(-n3c4ccccc4c4cc(-c5ccc6c(c5)c5ccccc5n6-c5cccc(-c6ccccc6)c5)ccc43)cc2)cc1]

### Phosphorescent Dopant

The phosphorescent dopant may include at least one transition metal as a central metal.

The phosphorescent dopant may include a monodentate ligand, a bidentate ligand, a tridentate ligand, a tetradentate ligand, a pentadentate ligand, a hexadentate ligand, or any combination thereof.

The phosphorescent dopant may be electrically neutral.

In one or more embodiments, the phosphorescent dopant may include an organometallic compound represented by Formula 401:

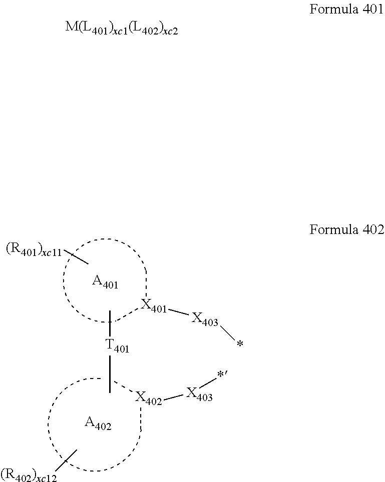

wherein, in Formulae 401 and 402,
M may be a transition metal (for example, iridium (Ir), platinum (Pt), palladium (Pd), osmium (Os), titanium (Ti), gold (Au), hafnium (Hf), europium (Eu), terbium (Tb), rhodium (Rh), rhenium (Re), or thulium (Tm)),
L₄₀₁ may be a ligand represented by Formula 402, and xc1 is 1, 2, or 3, wherein, when xc1 is 2 or more, two or more of L₄₀₁ may be identical to or different from each other,
L₄₀₂ may be an organic ligand, and xc2 may be 0, 1, 2, 3, or 4, wherein, when xc2 is 2 or more, two or more of L₄₀₂ may be identical to or different from each other,
X₄₀₁ and X₄₀₂ may each independently be nitrogen or carbon,
ring A₄₀₁ and ring A₄₀₂ may each independently be a C₃-C₆₀ carbocyclic group or a C₁-C₆₀ heterocyclic group,
T₄₀₁ may be a single bond, *—O—*′, *—S—*, *—C(═O)—*′, *—N(Q₄₁₁)—*′, *—C(Q₄₁₁)(Q₄₁₂)—*′, *—C(Q₄₁₁)=C(Q₄₁₂)—*′, *—C(Q₄₁₁)=*′, or *=C=*′,
X₄₀₃ and X₄₀₄ may each independently be a chemical bond (for example, a covalent bond or a coordinate bond), O, S, N(Q₄₁₃), B(Q₄₁₃), P(Q₄₁₃), C(Q₄₁₃)(Q₄₁₄), or Si(Q₄₁₃) (Q₄₁₄),
Q₄₁₁ to Q₄₁₄ are each as described in connection with Q₁ herein,
R₄₀₁ and R₄₀₂ may each independently be hydrogen, deuterium, —F, —Cl, —Br, —I, a hydroxyl group, a cyano group, a nitro group, a C₁-C₂₀ alkyl group that is unsubstituted or substituted with at least one R₁₀ₐ, a C₁-C₂₀ alkoxy group that is unsubstituted or substituted with at least one R₁₀ₐ, a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₄₀₁)(Q₄₀₂)(Q₄₀₃), —N(Q₄₀₁)(Q₄₀₂), —B(Q₄₀₁)(Q₄₀₂), —C(═O)(Q₄₀₁), —S(═O)₂(Q₄₀₁), or —P(═O)(Q₄₀₁)(Q₄₀₂),
Q₄₀₁ to Q₄₀₃ are each as described in connection with Q₁ herein,
xc11 and xc12 may each independently be an integer selected from 0 to 10, and
* and *′ in Formula 402 each indicate a binding site to M in Formula 401.

In one or more embodiments, in Formula 402, i) X₄₀₁ may be nitrogen, and X₄₀₂ may be carbon, or ii) each of X₄₀₁ and X₄₀₂ may be nitrogen.

In one or more embodiments, when xc1 in Formula 401 is 2 or more, two ring A₄₀₁ among two or more of L₄₀₁ may be optionally linked together via T₄₀₂, which is a linking group, and two ring A₄₀₂ among two or more of L₄₀₁ may be optionally linked together via T₄₀₃, which is a linking group (see Compounds PD1 to PD4 and PD7). T₄₀₂ and T₄₀₃ are each as described in connection with T₄₀₁ herein.

L₄₀₂ in Formula 401 may be an organic ligand. In one or more embodiments, L₄₀₂ may include a halogen group, a diketone group (for example, an acetylacetonate group), a carboxylic acid group (for example, a picolinate group), —C(═O), an isonitrile group, a —CN group, a phosphorus group (for example, a phosphine group, a phosphite group, and/or the like), or any combination thereof.

The phosphorescent dopant may include, for example, at least one of Compounds PD1 to PD39, or any combination thereof:

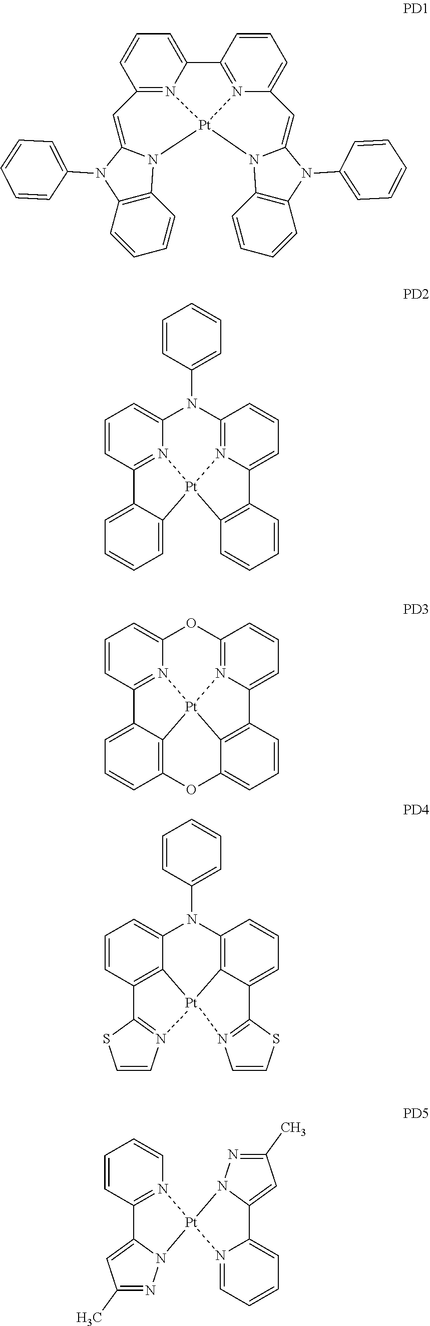

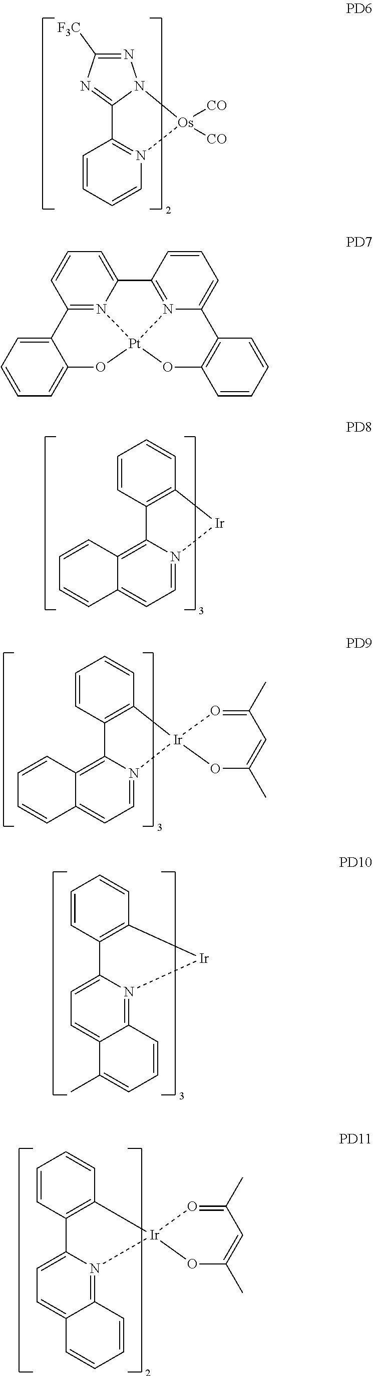

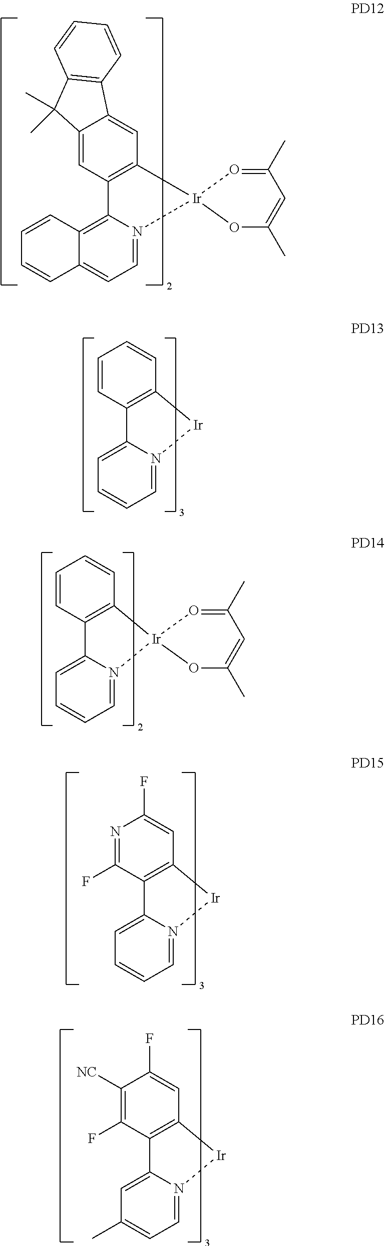

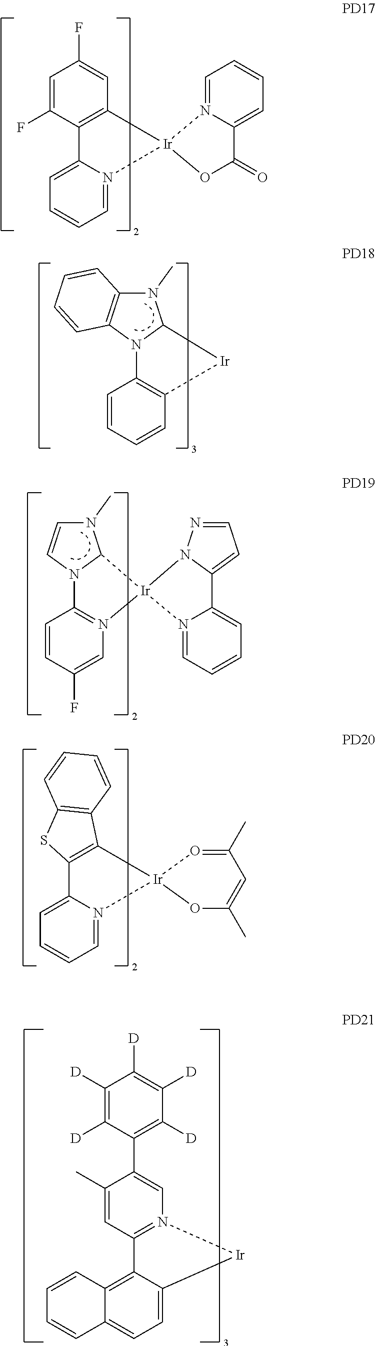

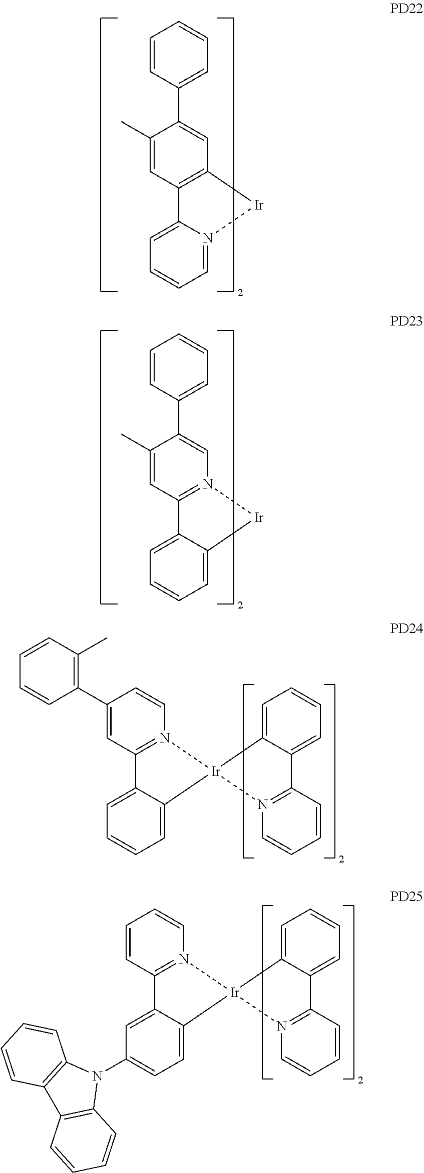

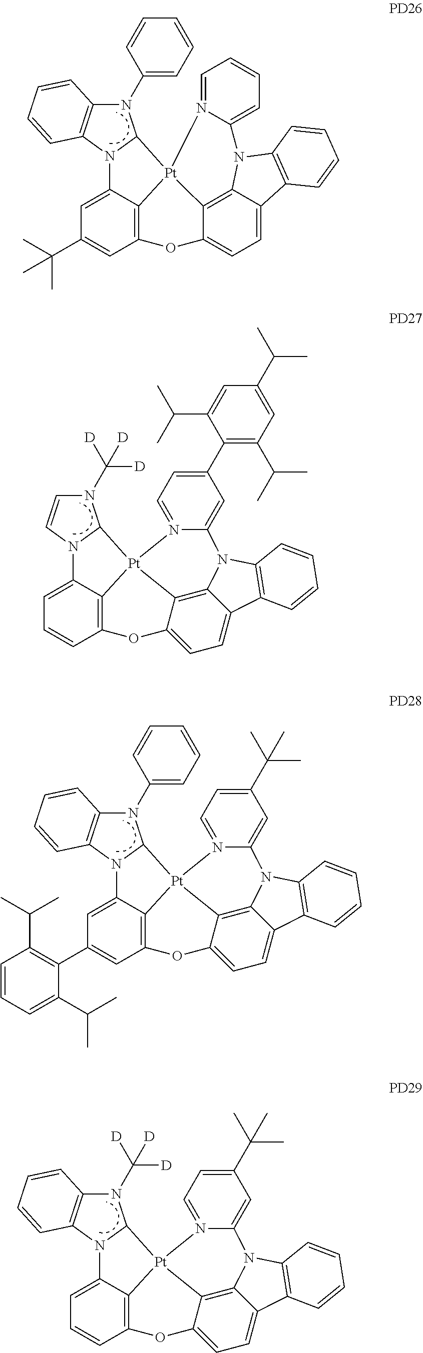

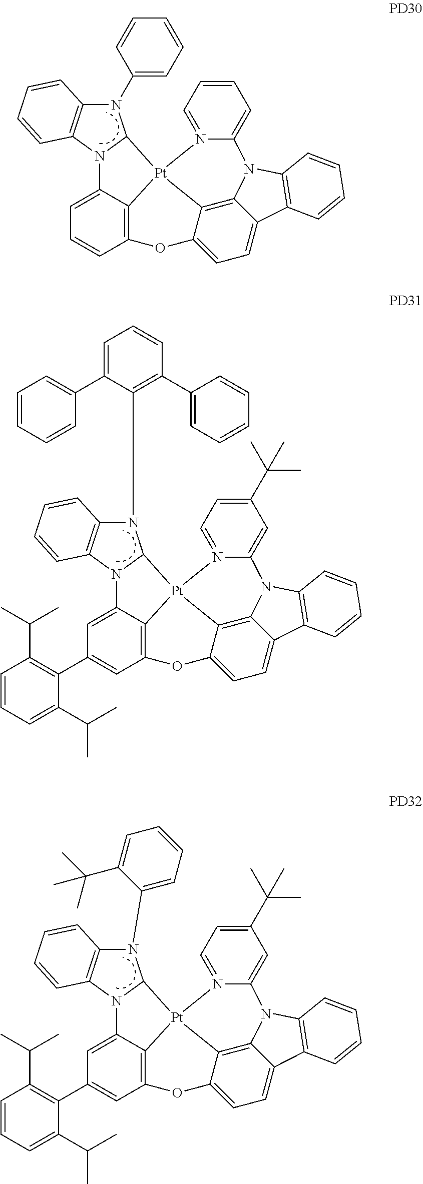

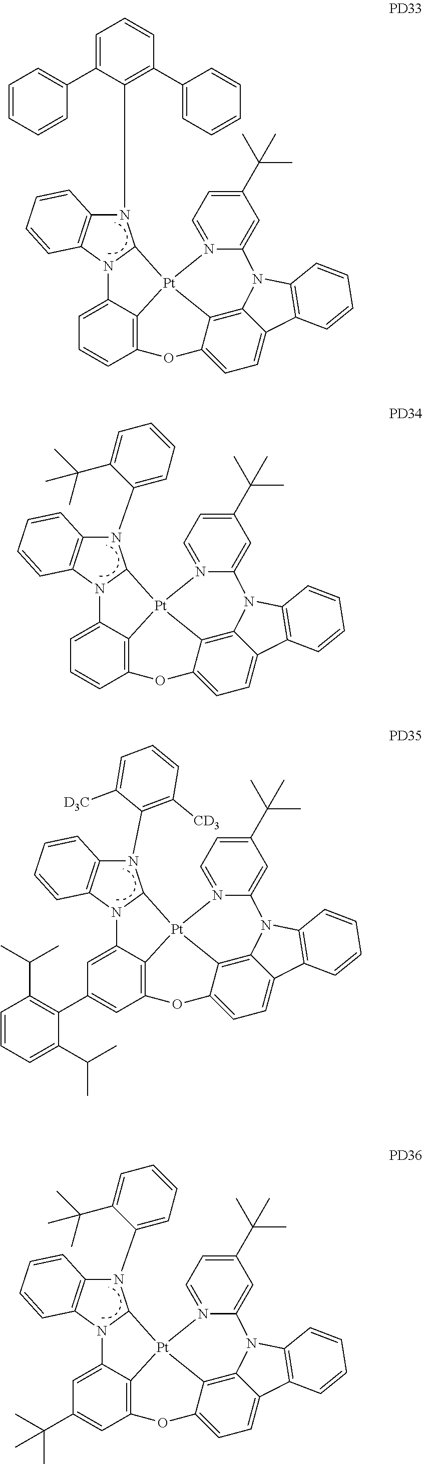

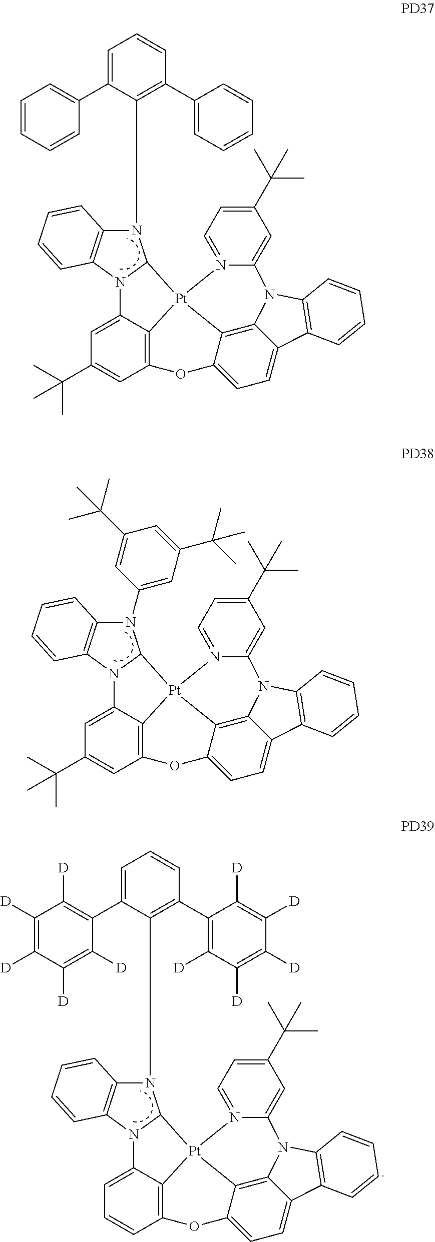

### Fluorescent Dopant

The fluorescent dopant may include an amine group-containing compound, a styryl group-containing compound, or any combination thereof.
For example, the fluorescent dopant may include a compound represented by Formula 501:

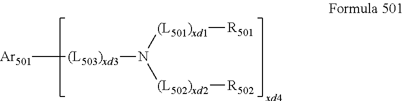

wherein, in Formula 501,
Ar₅₀₁, L₅₀₁ to L₅₀₃, R₅₀₁, and R₅₀₂ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xd1 to xd3 may each independently be 0, 1, 2, or 3, and
xd4 may be 1, 2, 3, 4, 5, or 6.

In one or more embodiments, Ar₅₀₁ in Formula 501 may be a condensed cyclic group (for example, an anthracene group, a chrysene group, a pyrene group, and/or the like) in which three or more monocyclic groups are condensed together.

In one or more embodiments, xd4 in Formula 501 may be 2.

In one or more embodiments, the fluorescent dopant may include: at least one of Compounds FD1 to FD37; DPVBi; DPAVBi; or any combination thereof:

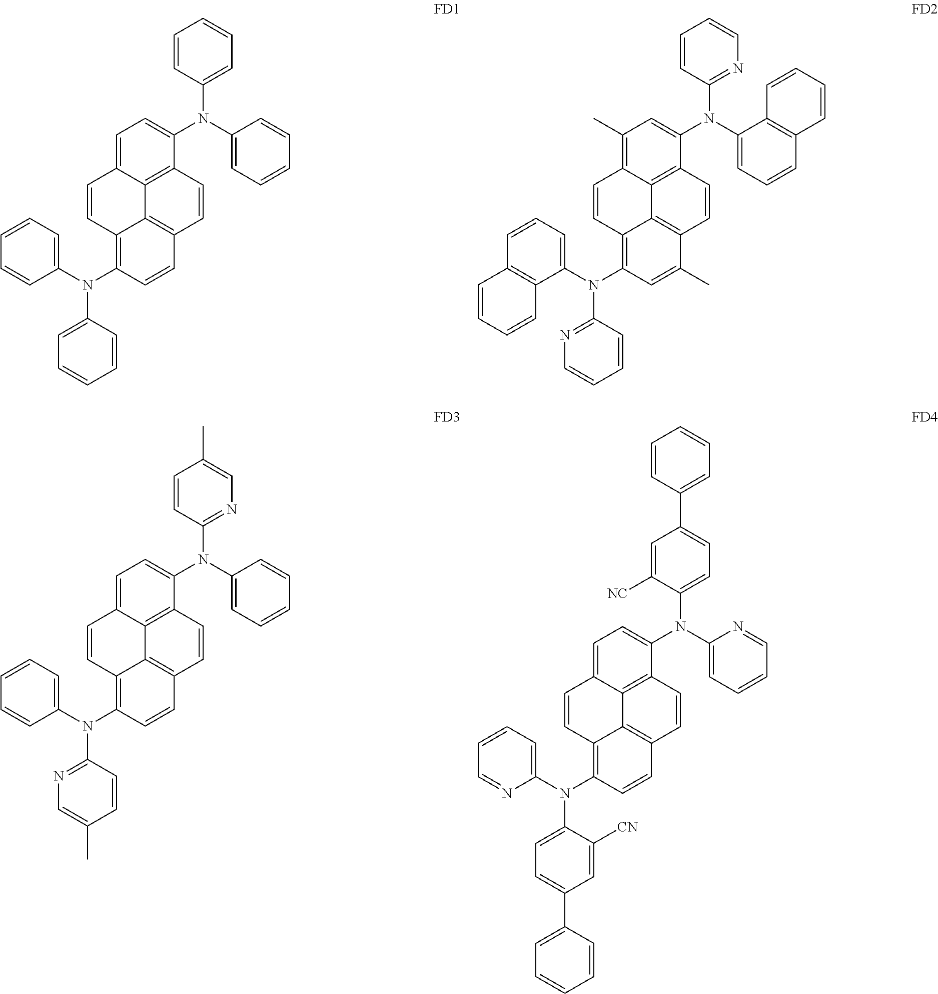 [SMILES: c1ccc(N(c2ccccc2)c2ccc3ccc4c(N(c5ccccc5)c5ccccc5)ccc5ccc2c3c54)cc1 ; Cc1cc(N(c2ccccn2)c2cccc3ccccc23)c2ccc3c(C)cc(N(c4ccccn4)c4cccc5ccccc45)c4ccc1c2c34 ; Cc1ccc(N(c2ccccc2)c2ccc3ccc4c(N(c5ccccc5)c5ccc(C)cn5)ccc5ccc2c3c54)nc1 ; N#Cc1cc(-c2ccccc2)ccc1N(c1ccccn1)c1ccc2ccc3c(N(c4ccccn4)c4ccc(-c5ccccc5)cc4C#N)ccc4ccc1c2c43]

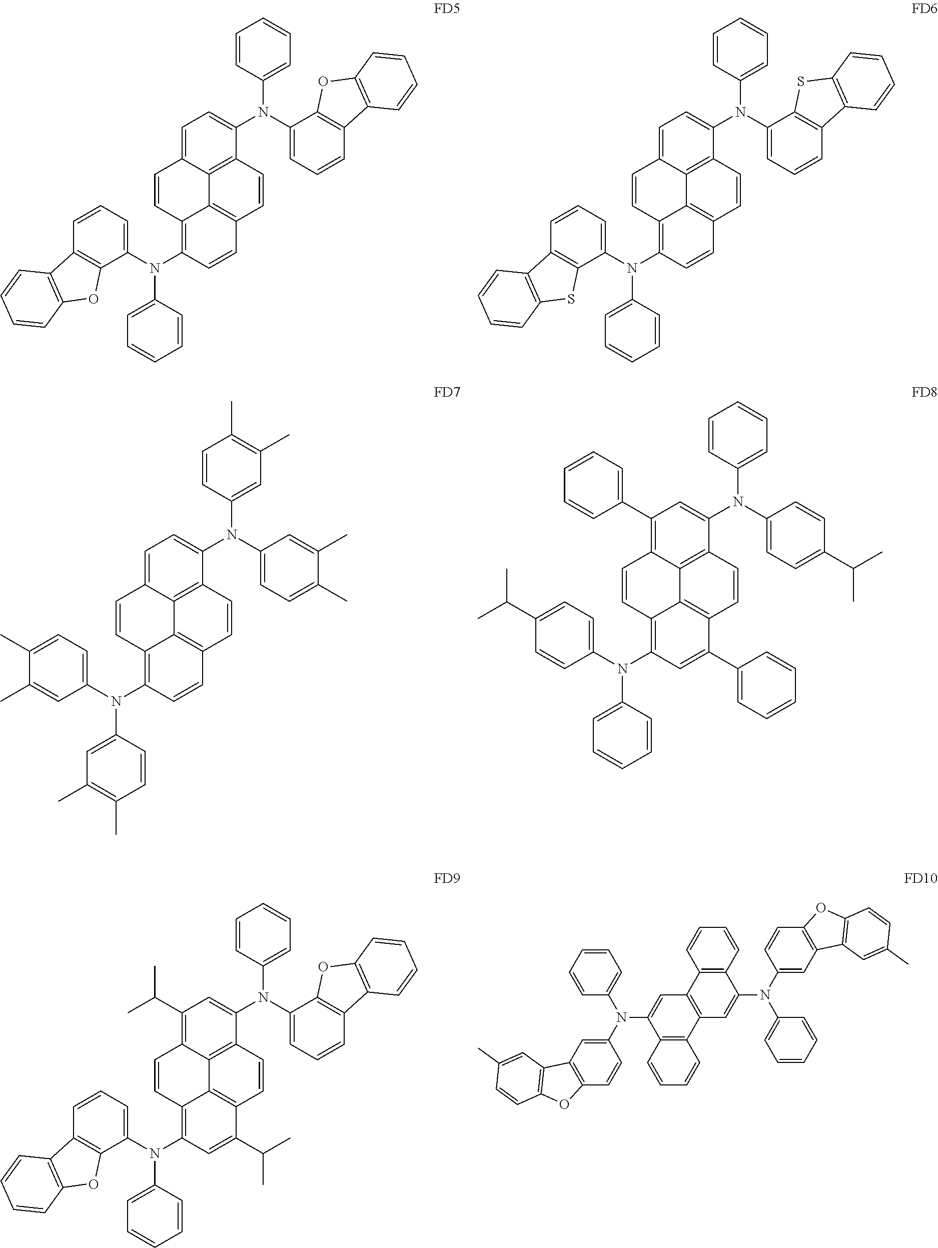

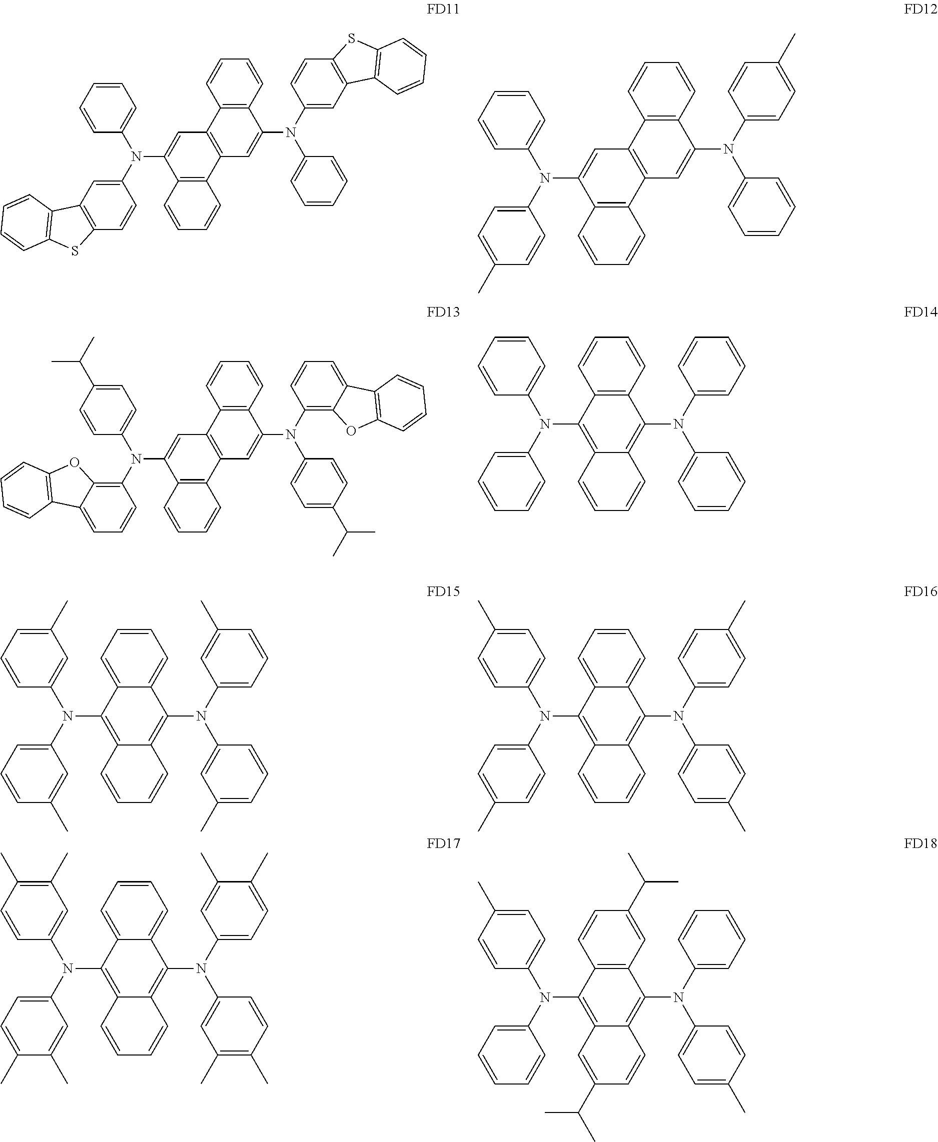

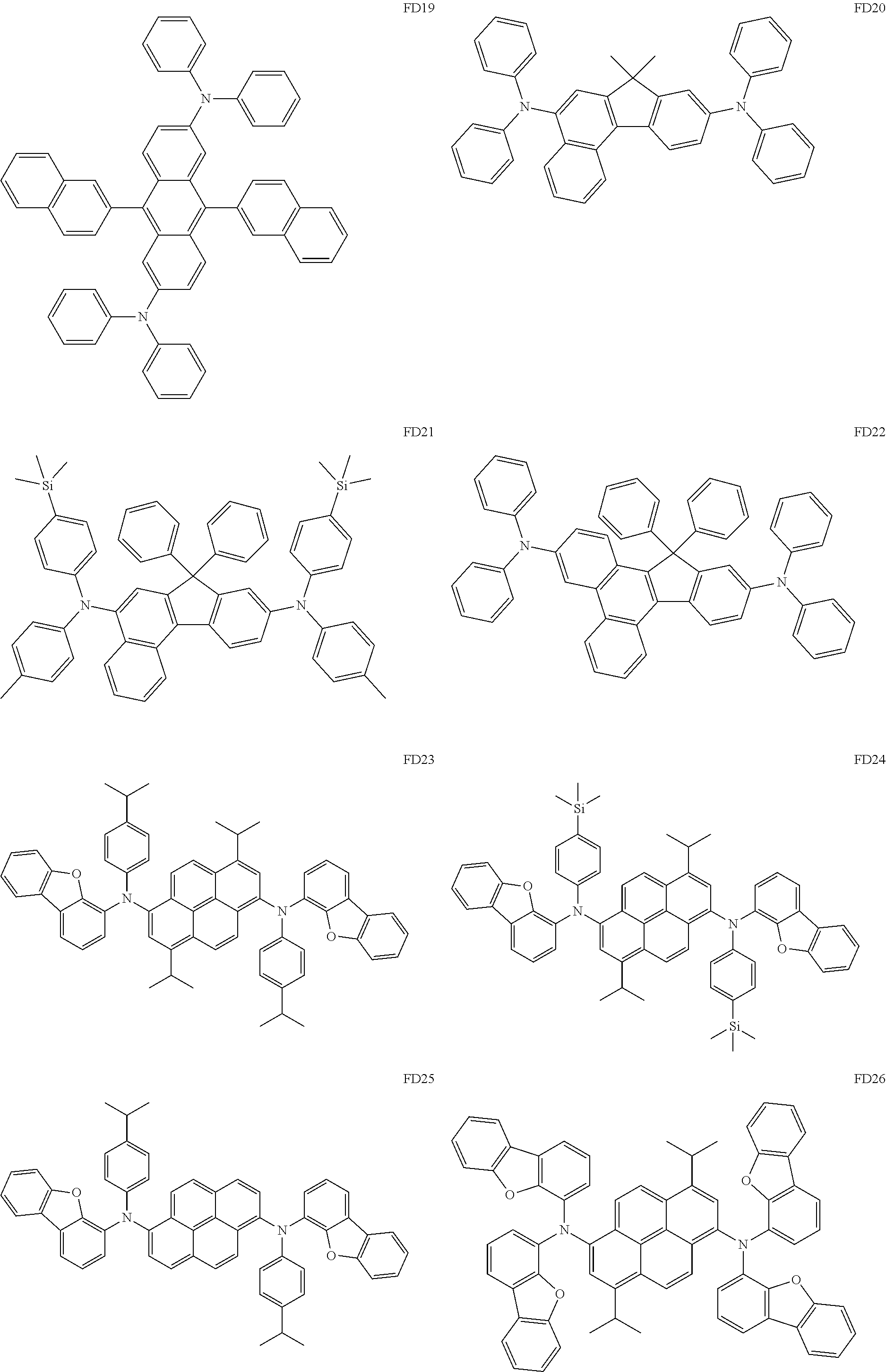

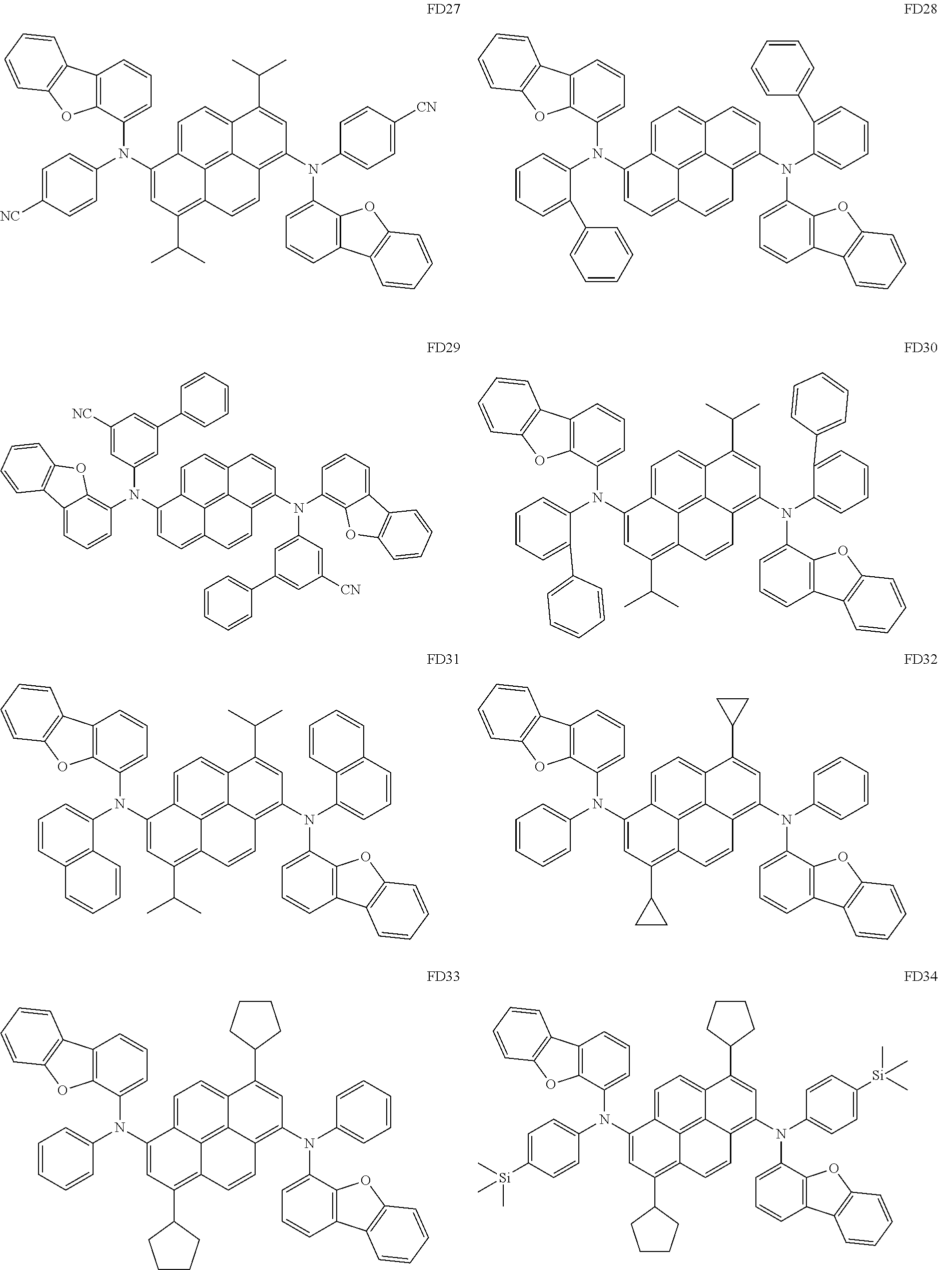

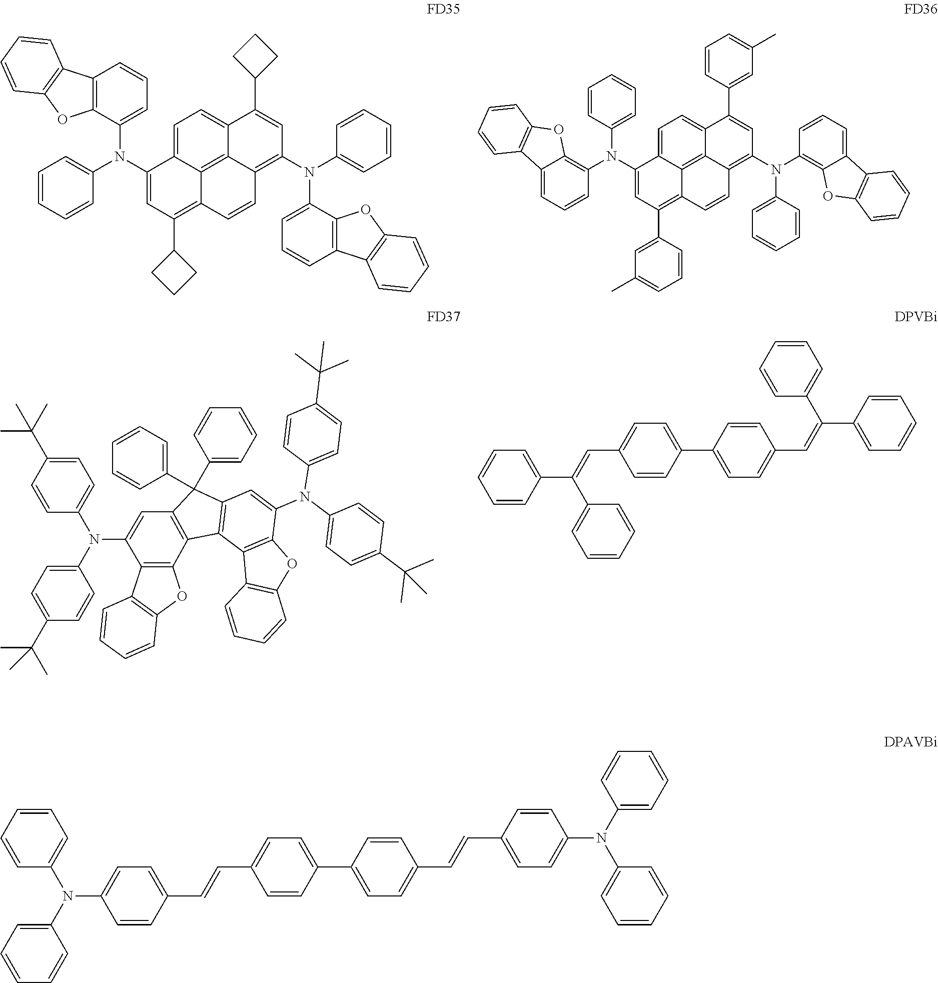

### Delayed Fluorescence Material

The emission layer may include a delayed fluorescence material.

Herein, the delayed fluorescence material may be selected from compounds capable of emitting delayed fluorescence based on a delayed fluorescence emission mechanism.

The delayed fluorescence material included in the emission layer may act as a host or a dopant depending on the type or kind of other materials included in the emission layer.

In one or more embodiments, a difference between a triplet energy level (eV) of the delayed fluorescence material and a singlet energy level (eV) of the delayed fluorescence material may be at least about 0 eV and not more than about 0.5 eV. When the difference between the triplet energy level (eV) of the delayed fluorescence material and the singlet energy level (eV) of the delayed fluorescence material is satisfied within the range described, up-conversion from the triplet state to the singlet state of the delayed fluorescence materials may effectively occur, and thus, the light-emitting device 10 may have improved luminescence efficiency.

In one or more embodiments, the delayed fluorescence material may include: i) a material including at least one electron donor (for example, a π electron-rich C₃-C₆₀ cyclic group such as a carbazole group) and at least one electron acceptor (for example, a sulfoxide group, a cyano group, a π electron-deficient nitrogen-containing C₁-C₆₀ cyclic group, and/or the like); and/or ii) a material including a C₈-C₆₀ polycyclic group including at least two cyclic groups that are condensed with each other while sharing boron (B).

Examples of the delayed fluorescence material may include at least one of Compounds DF1 to DF14 and/or D1:

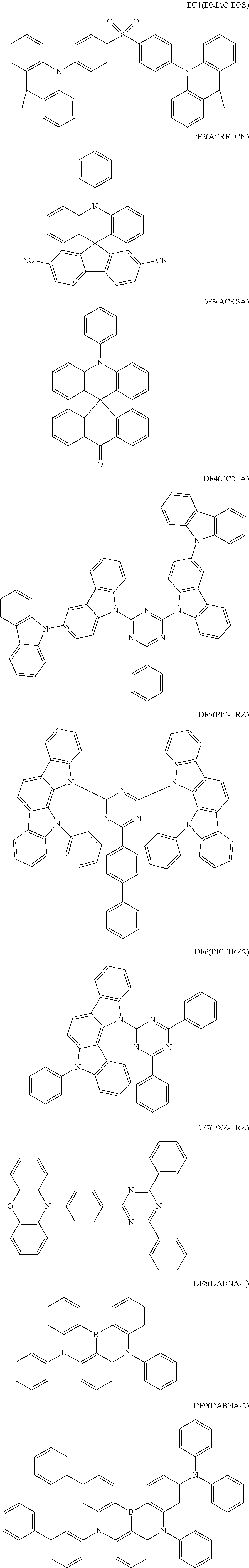

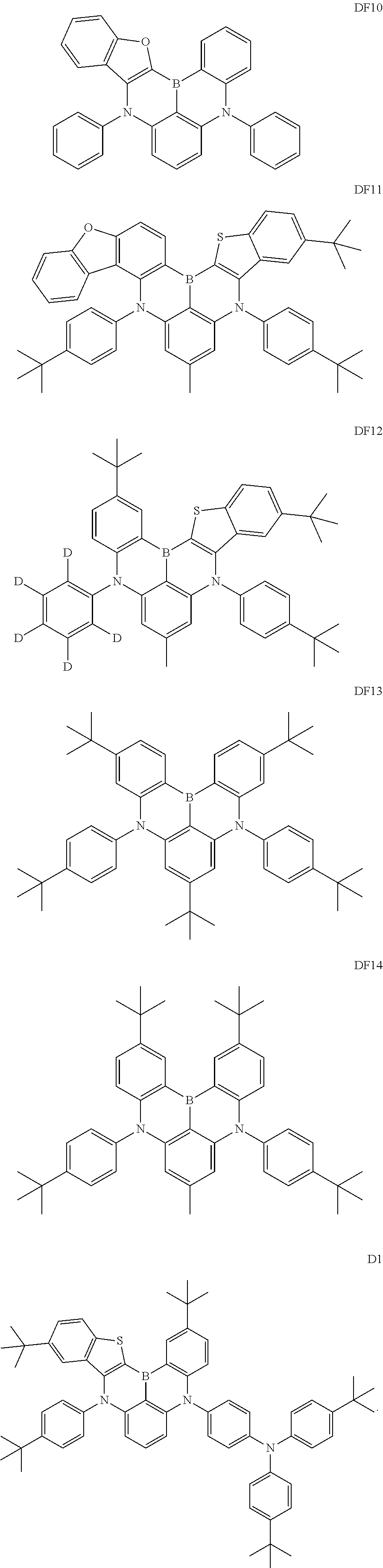

### Quantum Dot

The emission layer may include quantum dots.

Herein, quantum dots refer to crystals of a semiconductor compound. Quantum dots may be to emit (e.g., configured to emit) light of one or more suitable emission wavelengths depending on the size of crystals. Quantum dots may be to emit (e.g., configured to emit) light of one or more suitable emission wavelengths by adjusting a ratio of elements constituting the quantum dots.

The diameter of the quantum dots may be, for example, in a range of about 1 nanometer (nm) to about 10 nm.

The quantum dot may be synthesized by a wet chemical process, a metal organic chemical vapor deposition process, a molecular beam epitaxy process, or any process similar thereto.

The wet chemical process is a method including mixing a precursor material with an organic solvent and then growing quantum dot particle crystals. When the crystal grows, the organic solvent naturally acts as a dispersant coordinated on the surface of the quantum dot crystal and controls the growth of the crystal so that the growth of quantum dot particles can be controlled or selected through a process which costs lower, and is easier than vapor deposition methods, such as metal organic chemical vapor deposition (MOCVD) or molecular beam epitaxy (MBE).

The quantum dot may include: a Group II-VI semiconductor compound; a Group III-V semiconductor compound; a Group III-VI semiconductor compound; a Group I-III-VI semiconductor compound; a Group IV-VI semiconductor compound; a Group IV element or compound; or any combination thereof.

Examples of the Group II-VI semiconductor compound may include: a binary compound, such as CdS, CdSe, CdTe, ZnS, ZnSe, ZnTe, ZnO, HgS, HgSe, HgTe, MgSe, or MgS; a ternary compound, such as CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, or MgZnS; a quaternary compound, such as CdZnSeS, CdZnSeTe, CdZnSTe, CdHgSeS, CdHgSeTe, CdHgSTe, HgZnSeS, HgZnSeTe, or HgZnSTe; or any combination thereof.

Examples of the Group III-V semiconductor compound may include: a binary compound, such as GaN, GaP, GaAs, GaSb, AlN, AlP, AlAs, AlSb, InN, InP, InAs, or InSb; a ternary compound, such as GaNP, GaNAs, GaNSb, GaPAs, GaPSb, AlNP, AlNAs, AlNSb, AlPAs, AlPSb, InGaP, InNP, InAlP, InNAs, InNSb, InPAs, or InPSb; a quaternary compound, such as GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, or InAlPSb; or any combination thereof. In one or more embodiments, the Group III-V semiconductor compound may further include a Group II element. Examples of the Group III-V semiconductor compound further including the Group II element may include InZnP, InGaZnP, and/or InAlZnP.

Examples of the Group III-VI semiconductor compound may include: a binary compound, such as GaS, Ga₂Se₃, GaTe, InS, InSe, In₂S₃, In₂Se₃, InTe, and/or the like; a ternary compound, such as InGaS₃, InGaSe₃, etc; or any combination thereof.

Examples of the Group I-III-VI semiconductor compound may include: a ternary compound, such as AgInS, AgInS₂, AgInSe₂, AgGaS, AgGaS₂, AgGaSe₂, CuInS, CuInS₂, CuInSe₂, CuGaS₂, CuGaSe₂, CuGaO₂, AgGaO₂, AgAlO₂, and/or the like; a quaternary compound, such as AgInGaS₂, AgInGaSe₂, and/or the like; or any combination thereof.

Examples of the Group IV-VI semiconductor compound may include: a binary compound, such as SnS, SnSe, SnTe, PbS, PbSe, or PbTe; a ternary compound, such as SnSeS, SnSeTe, SnSTe, PbSeS, PbSeTe, PbSTe, SnPbS, SnPbSe, or SnPbTe; a quaternary compound, such as SnPbSSe, SnPbSeTe, or SnPbSTe; or any combination thereof.

Examples of the Group IV element or compound may include: a single element, such as Si, Ge, and/or the like; a binary compound, such as SiC, SiGe, and/or the like; or any combination thereof.

Each element included in a multi-element compound, such as the binary compound, the ternary compound, and the quaternary compound, may be present at a substantially uniform concentration or non-substantially uniform concentration in a particle. For example, the formulae refer to types (kinds) of elements included in the compound, wherein the element ratios in the compound may vary. For example, AgInGaS₂ may refer to AgInₓGa₁₋ₓS₂ (where x is a real number between 0 and 1).

In one or more embodiments, the quantum dot may have a single structure in which the concentration of each element in the quantum dot is substantially uniform, or may have a core-shell dual structure. In one or more embodiments, a material included in the core and a material included in the shell may be different from each other.

The shell of the quantum dot may act as a protective layer which prevents chemical denaturation of the core to maintain semiconductor characteristics, and/or as a charging layer which imparts electrophoretic characteristics to the quantum dot. The shell may be a single layer or a multilayer. The interface between the core and the shell may have a concentration gradient in which the concentration of an element existing in the shell decreases toward the center of the core.

Examples of the shell of the quantum dot may be an oxide of metal, or non-metal, a semiconductor compound, or any combination thereof. Examples of the oxide of metal or non-metal may include: a binary compound, such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO; a ternary compound, such as MgAl₂O₄, CoFe₂O₄, NiFe₂O₄, or CoMn₂O₄; or any combination thereof. Examples of the semiconductor compound may include, as described herein, a Group II-VI semiconductor compound; a Group III-V semiconductor compound; a Group III-VI semiconductor compound; a Group I-III-VI semiconductor compound; a Group IV-VI semiconductor compound; or any combination thereof. For example, the semiconductor compound may include CdS, CdSe, CdTe, ZnS, ZnSe, ZnTe, ZnSeS, ZnTeS, GaAs, GaP, GaS, GaSe, AgGaS, AgGaS₂, GaSb, HgS, HgSe, HgTe, InAs, InP, InGaP, InSb, AlAs, AlP, AlSb, or any combination thereof.

Each element included in the multi-element compound such as the binary compound and the ternary compound may be present in the particle at a substantially uniform or non-substantially uniform concentration. For example, the formulae refer to types (kinds) of elements included in the compound, wherein the element ratios in the compound may vary.

The quantum dot may have a full width at half maximum (FWHM) of the emission wavelength spectrum of about 45 nm or less, about 40 nm or less, or for example, about 30 nm or less, and in these ranges, color purity or color reproducibility may be increased. In some embodiments, because light emitted through the quantum dot is emitted in all directions, the wide viewing angle may be improved.

In some embodiments, the quantum dot may be in the form of spherical, pyramidal, multi-arm, or cubic nanoparticles, nanotubes, nanowires, nanofibers, or nanoplate particles.

Because the energy band gap may be controlled or selected by adjusting the size of the quantum dots or the ratio of elements in the quantum dot compound, light of one or more suitable wavelengths may be obtained from the quantum dot-containing emission layer. Therefore, by utilizing the aforementioned quantum dots (utilizing quantum dots of different sizes or having different element ratios in the quantum dot compound), a light-emitting device emitting light of one or more suitable wavelengths may be implemented. In more detail, the control of the size of the quantum dots or the ratio of elements in a quantum dot compound may be selected to emit red light, green light, and/or blue light. In some embodiments, the quantum dots may be configured to emit white light by combination of light of one or more suitable colors.

### Electron Transport Region 140

The electron transport region may have: i) a single-layered structure including (e.g., consisting of) a single layer including (e.g., consisting of) a single material; ii) a single-layered structure including (e.g., consisting of) a single layer including multiple different materials; or iii) a multilayer structure including multiple layers including multiple different materials.

The electron transport region may include a buffer layer, a hole blocking layer, an electron control layer, an electron transport layer, an electron injection layer, or any combination thereof.

In one or more embodiments, the electron transport region may have an electron transport layer/electron injection layer structure, a hole blocking layer/electron transport layer/electron injection layer structure, an electron control layer/electron transport layer/electron injection layer structure, or a buffer layer/electron transport layer/electron injection layer structure, wherein constituent layers of each structure are stacked in the stated order from the emission layer.

In one or more embodiments, the electron transport region (for example, a buffer layer, a hole blocking layer, an electron control layer, or an electron transport layer in the electron transport region) may include a metal-free compound including at least one π electron-deficient nitrogen-containing C₁-C₆₀ cyclic group.

In one or more embodiments, the electron transport region may include a compound represented by Formula 601.

[Ar₆₀₁]ₓₑ₁₁-[(L₆₀₁)ₓₑ₁-R₆₀₁]ₓₑ₂₁. Formula 601

In Formula 601,
Ar₆₀₁ and L₆₀₁ may each independently be a C₃-C₆₀ carbocyclic group that is unsubstituted or substituted with at least one R₁₀ₐ or a C₁-C₆₀ heterocyclic group that is unsubstituted or substituted with at least one R₁₀ₐ,
xe11 may be 1, 2, or 3,
xe1 may be 0, 1, 2, 3, 4, or 5,
R₆₀₁ may be a C₃-C₆₀ carbocyclic group that is unsubstituted or substituted with at least one R₁₀ₐ, a C₁-C₆₀ heterocyclic group that is unsubstituted or substituted with at least one R₁₀ₐ, —Si(Q₆₀₁)(Q₆₀₂)(Q₆₀₃), —C(═O)(Q₆₀₁), —S(═O)₂(Q₆₀₁), or —P(═O)(Q₆₀₁)(Q₆₀₂),
Q₆₀₁ to Q₆₀₃ are each as described in connection with Q₁ herein,
xe21 may be 1, 2, 3, 4, or 5, and
at least one of Ar₆₀₁, L₆₀₁, and R₆₀₁ may each independently be a π electron-deficient nitrogen-containing C₁-C₆₀ cyclic group that is unsubstituted or substituted with at least one R₁₀ₐ.

In one or more embodiments, when xe11 in Formula 601 is 2 or more, two or more of Ar₆₀₁ may be linked together via a single bond.

In one or more embodiments, Ar₆₀₁ in Formula 601 may be an anthracene group that is unsubstituted or substituted with at least one R₁₀ₐ.

In one or more embodiments, the electron transport region may include a compound represented by Formula 601-1:

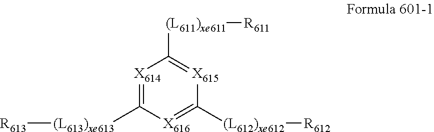

wherein, in Formula 601-1,
X₆₁₄ may be N or C(R₆₁₄), X₆₁₅ may be N or C(R₆₁₅), X₆₁₆ may be N or C(R₆₁₆), and at least one of X₆₁₄ to X₆₁₆ may be N,
L₆₁₁ to L₆₁₃ are each as described in connection with L₆₀₁,
xe611 to xe613 are each as described in connection with xe1,
R₆₁₁ to R₆₁₃ are each as described in connection with R₆₀₁, and
R₆₁₄ to R₆₁₆ may each independently be hydrogen, deuterium, —F, —Cl, —Br, —I, a hydroxyl group, a cyano group, a nitro group, a C₁-C₂₀ alkyl group, a C₁-C₂₀ alkoxy group, a C₃-C₆₀ carbocyclic group that is unsubstituted or substituted with at least one R₁₀ₐ, or a C₁-C₆₀ heterocyclic group that is unsubstituted or substituted with at least one R₁₀ₐ.

In one or more embodiments, xe1 and xe611 to xe613 in Formulae 601 and 601-1 may each independently be 0, 1, or 2.

The electron transport region may include at least one of Compounds ET1 to ET45, 2,9-dimethyl-4,7-diphenyl-1,10-phenanthroline (BCP), 4,7-diphenyl-1,10-phenanthroline (Bphen), Alq₃, BAlq, TAZ, NTAZ, or any combination thereof:

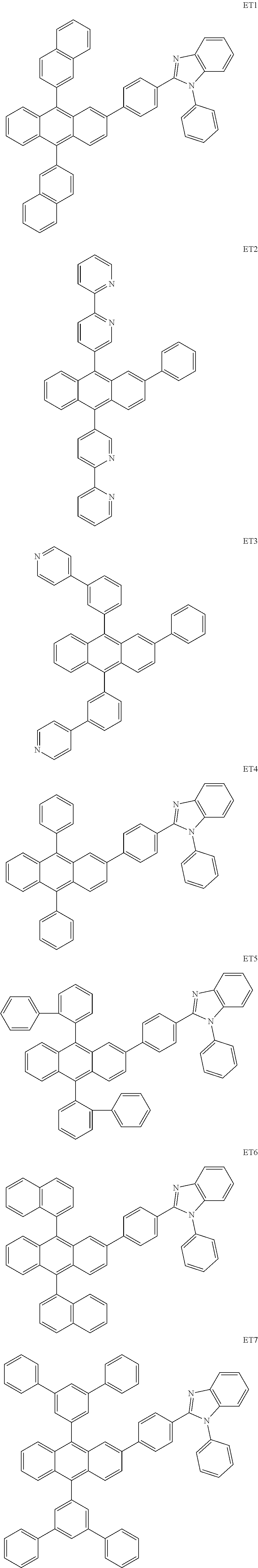

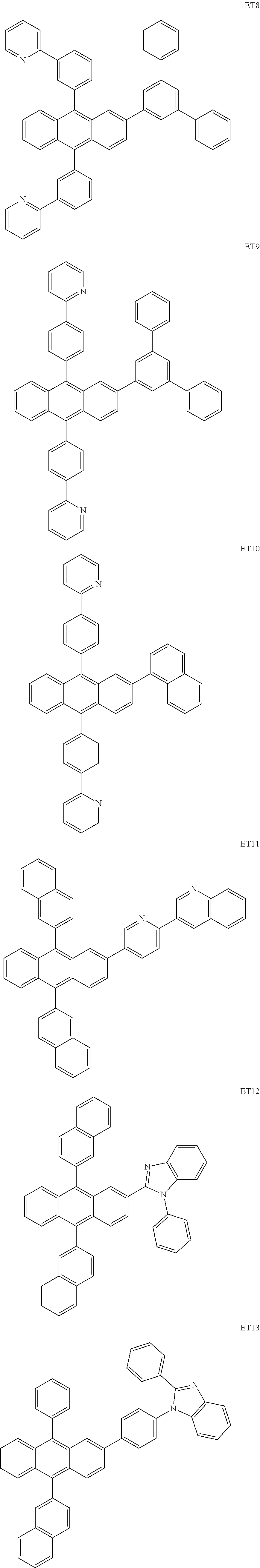

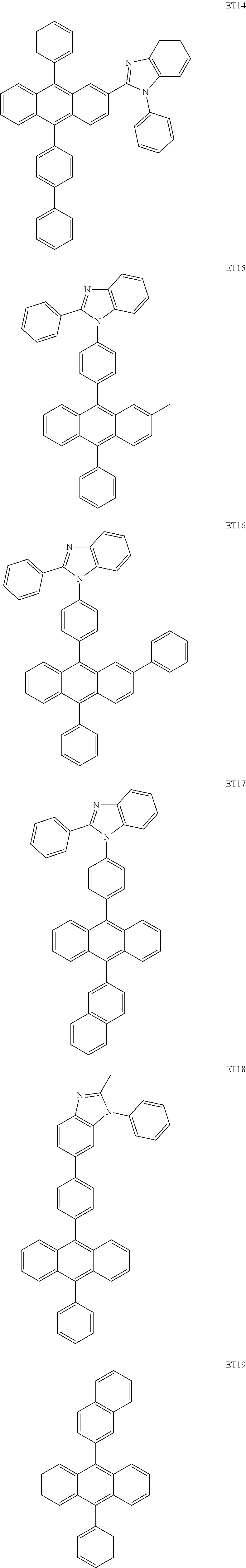

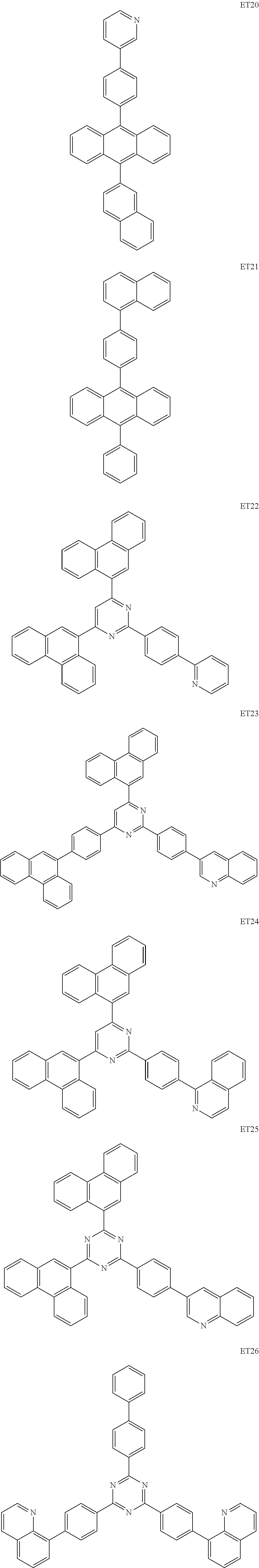

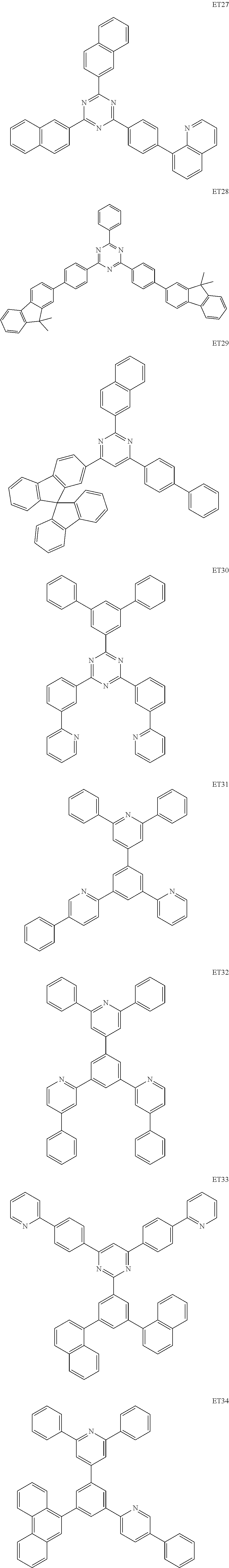

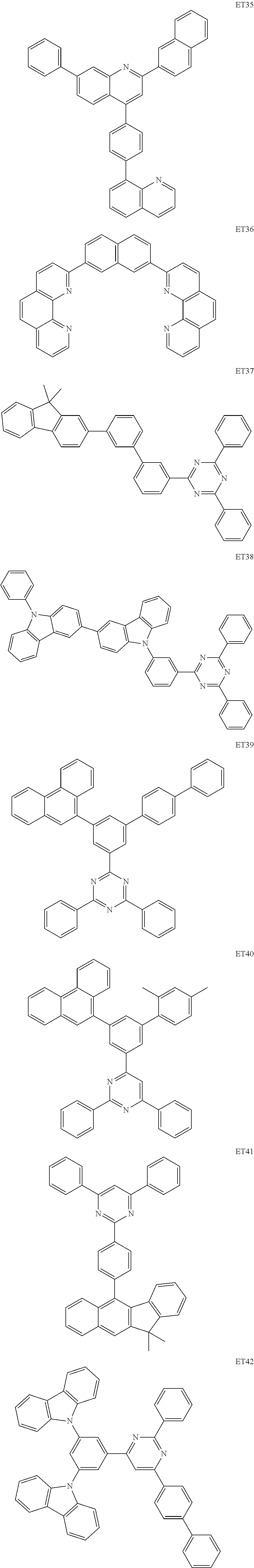

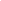

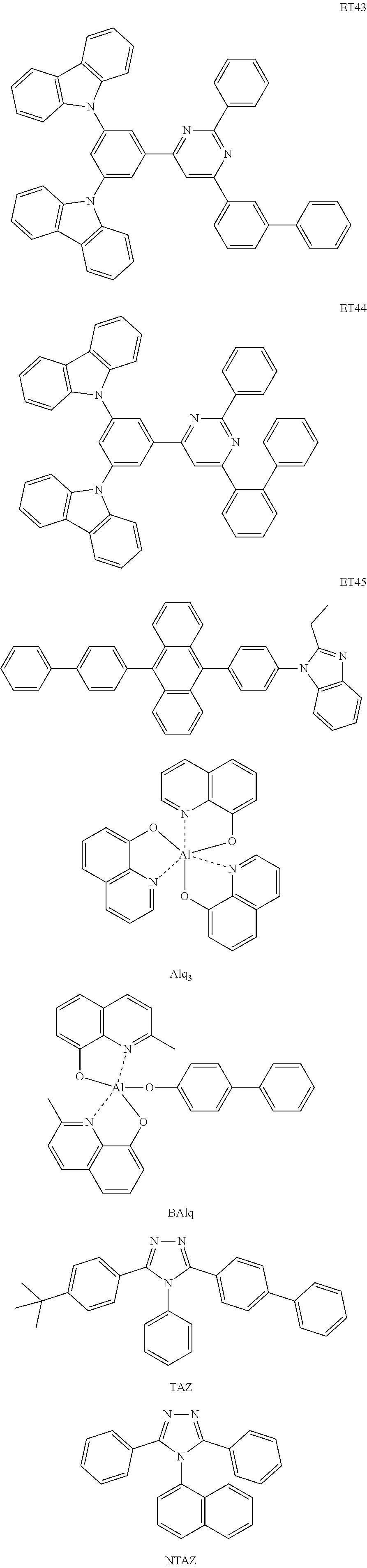

The thickness of the electron transport region may be about 100 Å to about 5,000 Å, for example, about 160 Å to about 4,000 Å. When the electron transport region includes a buffer layer, a hole blocking layer, an electron control layer, an electron transport layer, or any combination thereof, the thickness of the buffer layer, the hole blocking layer, or the electron control layer may be about 20 Å to about 1,000 Å, for example, about 30 Å to about 300 Å, and the thickness of the electron transport layer may be about 100 Å to about 1,000 Å, for example, about 150 Å to about 500 Å. When the thickness of the buffer layer, the hole blocking layer, the electron control layer, the electron transport layer, and/or the electron transport region are within these ranges, satisfactory electron transporting characteristics may be obtained without a substantial increase in driving voltage.

The electron transport region (for example, the electron transport layer in the electron transport region) may further include, in addition to the materials described, a metal-containing material.

The metal-containing material may include an alkali metal complex, an alkaline earth metal complex, or any combination thereof. A metal ion of the alkali metal complex may be a Li ion, a Na ion, a K ion, a Rb ion, or a Cs ion, and a metal ion of the alkaline earth metal complex may be a Be ion, a Mg ion, a Ca ion, a Sr ion, or a Ba ion. A ligand coordinated with the metal ion of the alkali metal complex or the alkaline earth-metal complex may include a hydroxyquinoline, a hydroxyisoquinoline, a hydroxybenzoquinoline, a hydroxyacridine, a hydroxyphenanthridine, a hydroxyphenyloxazole, a hydroxyphenylthiazole, a hydroxyphenyloxadiazole, a hydroxyphenylthiadiazole, a hydroxyphenylpyridine, a hydroxyphenylbenzimidazole, a hydroxyphenylbenzothiazole, a bipyridine, a phenanthroline, a cyclopentadiene, or any combination thereof.

In one or more embodiments, the metal-containing material may include a Li complex. The Li complex may include, for example, Compound ET-D1 (LiQ) and/or ET-D2:

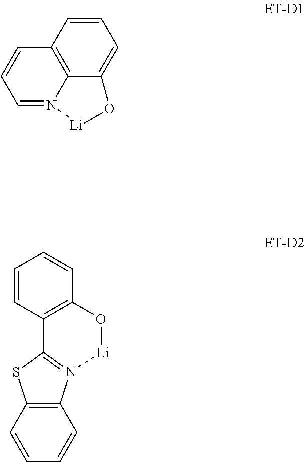

The electron transport region may include an electron injection layer that facilitates the injection of electrons from the second electrode 150. The electron injection layer may directly contact the second electrode 150.

The electron injection layer may have: i) a single-layered structure including (e.g., consisting of) a single layer including (e.g., consisting of) a single material; ii) a single-layered structure including (e.g., consisting of) a single layer including multiple different materials; or iii) a multilayer structure including multiple layers including multiple different materials.

The electron injection layer may include an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof.

The alkali metal may include Li, Na, K, Rb, Cs, or any combination thereof. The alkaline earth metal may include Mg, Ca, Sr, Ba, or any combination thereof. The rare earth metal may include Sc, Y, Ce, Tb, Yb, Gd, or any combination thereof.

The alkali metal-containing compound, the alkaline earth metal-containing compound, and the rare earth metal-containing compound may include oxides, halides (for example, fluorides, chlorides, bromides, iodides, and/or the like), or tellurides of the alkali metal, the alkaline earth metal, and the rare earth metal, or any combination thereof.

The alkali metal-containing compound may include: alkali metal oxides, such as Li₂O, Cs₂O, or K₂O; alkali metal halides, such as LiF, NaF, CsF, KF, LiI, NaI, CsI, or KI; or any combination thereof. The alkaline earth metal-containing compound may include an alkaline earth metal compound, such as BaO, SrO, CaO, BaₓSr₁₋ₓO (x is a real number satisfying 0<x<1), or BaₓCa₁₋ₓO (x is a real number satisfying 0<x<1). The rare earth metal-containing compound may include YbF₃, ScF₃, Sc₂O₃, Y₂O₃, Ce₂O₃, GdF₃, TbF₃, YbI₃, ScI₃, TbI₃, or any combination thereof. In one or more embodiments, the rare earth metal-containing compound may include lanthanide metal telluride. Examples of the lanthanide metal telluride may include LaTe, CeTe, PrTe, NdTe, PmTe, SmTe, EuTe, GdTe, TbTe, DyTe, HoTe, ErTe, TmTe, YbTe, LuTe, La₂Te₃, Ce₂Te₃, Pr₂Te₃, Nd₂Te₃, Pm₂Te₃, Sm₂Te₃, Eu₂Te₃, Gd₂Te₃, Tb₂Te₃, Dy₂Te₃, Ho₂Te₃, Er₂Te₃, Tm₂Te₃, Yb₂Te₃, and/or Lu₂Te₃.

The alkali metal complex, the alkaline earth-metal complex, and the rare earth metal complex may include i) at least one selected from among ions of the alkali metal, the alkaline earth metal, and/or the rare earth metal and ii) a ligand bonded to the metal ion (i.e., the selected metal ion), for example, hydroxyquinoline, hydroxyisoquinoline, hydroxybenzoquinoline, hydroxyacridine, hydroxyphenanthridine, hydroxyphenyloxazole, hydroxyphenylthiazole, hydroxyphenyloxadiazole, hydroxyphenylthiadiazole, hydroxyphenylpyridine, hydroxyphenyl benzimidazole, hydroxyphenylbenzothiazole, bipyridine, phenanthroline, cyclopentadiene, or any combination thereof.

The electron injection layer may include (e.g., consist of) an alkali metal, an alkaline earth metal, a rare earth metal, an alkali metal-containing compound, an alkaline earth metal-containing compound, a rare earth metal-containing compound, an alkali metal complex, an alkaline earth metal complex, a rare earth metal complex, or any combination thereof, as described. In one or more embodiments, the electron injection layer may further include an organic material (for example, a compound represented by Formula 601).

In one or more embodiments, the electron injection layer may include (e.g., consist of) i) an alkali metal-containing compound (for example, alkali metal halide), and/or ii) a) an alkali metal-containing compound (for example, alkali metal halide); and b) an alkali metal, an alkaline earth metal, a rare earth metal, or any combination thereof. In one or more embodiments, the electron injection layer may be a KI:Yb co-deposited layer, an RbI:Yb co-deposited layer, a LiF:Yb co-deposited layer, and/or the like.

When the electron injection layer further includes an organic material, the alkali metal, the alkaline earth metal, the rare earth metal, the alkali metal-containing compound, the alkaline earth metal-containing compound, the rare earth metal-containing compound, the alkali metal complex, the alkaline earth-metal complex, the rare earth metal complex, or any combination thereof may be uniformly or non-uniformly dispersed in a matrix including the organic material.

The thickness of the electron injection layer may be about 1 Å to about 100 Å, and, for example, about 3 Å to about 90 Å. When the thickness of the electron injection layer is within the range as described, satisfactory electron injection characteristics may be obtained without a substantial increase in driving voltage.

### Second Electrode 150

The second electrode 140 may be provided on the electron transport region 140. The second electrode 150 may be a cathode, which is an electron injection electrode, and as a material for forming the second electrode 150, a metal, an alloy, an electrically conductive compound, or any combination thereof, each having a low work-function, may be utilized. The term “low work-function material” as utilized herein refers to a substance (e.g., a metal or metal alloy) that requires a relatively small, or low, amount of energy to emit electrons from its surface.

The second electrode 150 may include lithium (Li), silver (Ag), magnesium (Mg), aluminum (Al), aluminum-lithium (Al—Li ), calcium (Ca), magnesium-indium (Mg—In), magnesium-silver (Mg—Ag), ytterbium (Yb), silver-ytterbium (Ag—Yb), ITO, IZO, or any combination thereof. The second electrode 150 may be a transmissive electrode, a semi-transmissive electrode, or a reflective electrode.

The second electrode 150 may have a single-layer structure or a multilayer structure including multiple layers.

### Capping Layer

The first capping layer may be arranged outside (and, e.g., on) the first electrode 110, and/or the second capping layer may be arranged outside (and, e.g., on) the second electrode 150. In more detail, the light-emitting device 10 may have a structure in which the first capping layer, the first electrode 110, the interlayer, and the second electrode 150 are stacked in the stated order, a structure in which the first electrode 110, the interlayer, the second electrode 150, and the second capping layer are stacked in the stated order, or a structure in which the first capping layer, the first electrode 110, the interlayer, the second electrode 150, and the second capping layer are stacked in the stated order.

Light generated in the emission layer 130 of the light-emitting device 10 may be extracted toward the outside through the first electrode 110 which is a semi-transmissive electrode or a transmissive electrode, and the first capping layer. Light generated in the emission layer 130 of the light-emitting device 10 may be extracted toward the outside through the second electrode 150 which is a semi-transmissive electrode or a transmissive electrode, and the second capping layer.

The first capping layer and the second capping layer may increase external emission efficiency according to the principle of constructive interference. Accordingly, the light extraction efficiency of the light-emitting device 10 is increased, such that the luminescence efficiency of the light-emitting device 10 may be increased.

Each of the first capping layer and the second capping layer may include a material having a refractive index of greater than or equal to 1.2 (at 460 nm).

The first capping layer and the second capping layer may each independently be an organic capping layer including an organic material, an inorganic capping layer including an inorganic material, or an organic-inorganic composite capping layer including an organic material and an inorganic material.

At least one of the first capping layer and/or the second capping layer may each independently include a carbocyclic compound, a heterocyclic compound, an amine group-containing compound, a porphine derivative, a phthalocyanine derivative, a naphthalocyanine derivative, an alkali metal complex, an alkaline earth metal complex, or any combination thereof. The carbocyclic compound, the heterocyclic compound, and the amine group-containing compound may be optionally substituted with a substituent including O, N, S, Se, Si, F, Cl, Br, I, or any combination thereof. In one or more embodiments, at least one of the first capping layer and the second capping layer may each independently include an amine group-containing compound.

In one or more embodiments, at least one of the first capping layer and/or the second capping layer may each independently include a compound represented by Formula 201, a compound represented by Formula 202, or any combination thereof.

In one or more embodiments, at least one of the first capping layer and the second capping layer may each independently include at least one of Compounds HT28 to HT33, at least one of Compounds CP1 to CP6, p-NPB, or any combination thereof:

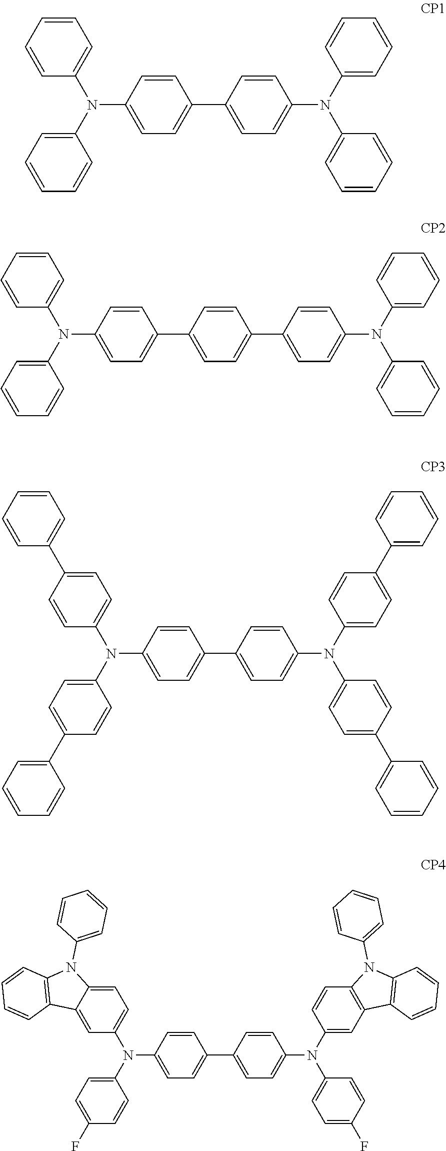 [SMILES: c1ccc(N(c2ccccc2)c2ccc(-c3ccc(N(c4ccccc4)c4ccccc4)cc3)cc2)cc1 ; c1ccc(N(c2ccccc2)c2ccc(-c3ccc(-c4ccc(N(c5ccccc5)c5ccccc5)cc4)cc3)cc2)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; Fc1ccc(N(c2ccc(-c3ccc(N(c4ccc(F)cc4)c4ccc5c(c4)c4ccccc4n5-c4ccccc4)cc3)cc2)c2ccc3c(c2)c2ccccc2n3-c2ccccc2)cc1]

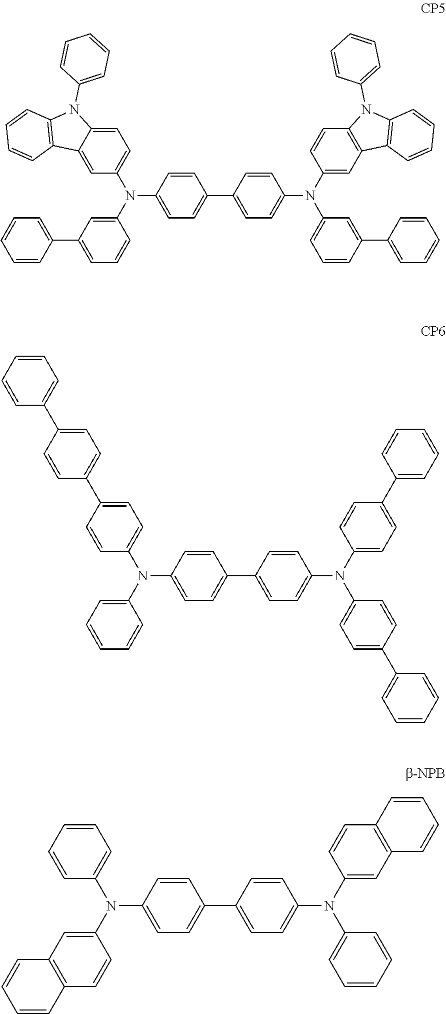 [SMILES: c1ccc(-c2cccc(N(c3ccc(-c4ccc(N(c5cccc(-c6ccccc6)c5)c5ccc6c(c5)c5ccccc5n6-c5ccccc5)cc4)cc3)c3ccc4c(c3)c3ccccc3n4-c3ccccc3)c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1]

### Film

The electronic apparatus may further include a film. The film may be, for example, an optical member (or a light control component) (for example, a color filter, a color conversion member, a capping layer, a light extraction efficiency enhancement layer, a selective light absorbing layer, a polarizing layer, a quantum dot-containing layer, and/or the like), a light-blocking member (for example, a light reflective layer, a light absorbing layer, and/or the like), and/or a protective member (for example, an insulating layer, a dielectric layer, and/or the like).

### Electronic Apparatus

The light-emitting device may be included in one or more suitable electronic apparatuses. For example, the electronic apparatus including the light-emitting device may be a display apparatus, an authentication apparatus, and/or the like.

The electronic apparatus (for example, a display apparatus) may further include, in addition to the light-emitting device, i) a color filter, ii) a color conversion layer, or iii) a color filter and a color conversion layer. The color filter and/or the color conversion layer may be arranged in at least one traveling direction of light emitted from the light-emitting device. For example, the light emitted from the light-emitting device may be blue light or white light. A detailed description of the light-emitting device is provided. In one or more embodiments, the color conversion layer may include quantum dots. The quantum dot may be, for example, a quantum dot as described herein.

The electronic apparatus may include a first substrate. The first substrate may include a plurality of subpixel areas, the color filter may include a plurality of color filter areas respectively corresponding to the subpixel areas, and the color conversion layer may include a plurality of color conversion areas respectively corresponding to the subpixel areas.

A pixel-defining film may be arranged among the subpixel areas to define each of the subpixel areas.

The color filter may further include a plurality of color filter areas and light-shielding patterns arranged among the color filter areas, and the color conversion layer may further include a plurality of color conversion areas and light-shielding patterns arranged among the color conversion areas.

The plurality of color filter areas (or the plurality of color conversion areas) may include a first area emitting first color light, a second area emitting second color light, and/or a third area emitting third color light, wherein the first color light, the second color light, and/or the third color light may have different maximum emission wavelengths. In one or more embodiments, the first color light may be red light, the second color light may be green light, and the third color light may be blue light. In one or more embodiments, the plurality of color filter areas (or the plurality of color conversion areas) may include quantum dots. In more detail, the first area may include red quantum dots, the second area may include green quantum dots, and the third area may not include (e.g., may exclude) quantum dots. A detailed description of the quantum dots is provided herein. The first area, the second area, and/or the third area may each further include a scatterer.

In one or more embodiments, the light-emitting device may be to emit (e.g., configured to emit) first light, the first area may be to absorb (e.g., configured to absorb) the first light to emit first-first color light, the second area may be to absorb (e.g., configured to absorb) the first light to emit second-first color light, and the third area may be to absorb (e.g., configured to absorb) the first light to emit third-first color light. In this case, the first-first color light, the second-first color light, and the third-first color light may have different maximum emission wavelengths. In more detail, the first light may be blue light, the first-first color light may be red light, the second-first color light may be green light, and the third-first color light may be blue light.

The electronic apparatus may further include a thin-film transistor, in addition to the light-emitting device as described. The thin-film transistor may include a source electrode, a drain electrode, and an activation layer, wherein any one of the source electrode or the drain electrode may be electrically connected to any one of the first electrode or the second electrode of the light-emitting device.

The thin-film transistor may further include a gate electrode, a gate insulating film, and/or the like.

The activation layer may include crystalline silicon, amorphous silicon, an organic semiconductor, an oxide semiconductor, and/or the like.

The electronic apparatus may further include a sealing portion for sealing the light-emitting device. The sealing portion may be arranged between the color filter and/or the color conversion layer and the light-emitting device. The sealing portion allows light from the light-emitting device to be extracted to the outside, and concurrently (e.g., simultaneously) prevents ambient air and moisture from penetrating into the light-emitting device. The sealing portion may be a sealing substrate including a transparent glass substrate or a plastic substrate. The sealing portion may be a thin-film encapsulation layer including at least one layer of an organic layer and/or an inorganic layer. When the sealing portion is a thin film encapsulation layer, the electronic apparatus may be flexible.

One or more suitable functional layers may be additionally provided on the sealing portion, in addition to the color filter and/or the color conversion layer, according to the utilize of the electronic apparatus. Examples of the functional layers may include a touch screen layer and a polarizing layer. The touch screen layer may be a pressure-sensitive touch screen layer, a capacitive touch screen layer, or an infrared touch screen layer. The authentication apparatus may be, for example, a biometric authentication apparatus that authenticates an individual by utilizing biometric information of a living body (for example, fingertips, pupils, and/or the like).

The authentication apparatus may further include, in addition to the light-emitting device as described, a biometric information collector.

The electronic apparatus may be applied to one or more suitable displays, light sources, lighting, personal computers (for example, a mobile personal computer), mobile phones, digital cameras, electronic organizers, electronic dictionaries, electronic game machines, medical instruments (for example, electronic thermometers, sphygmomanometers, blood glucose meters, pulse measurement devices, pulse wave measurement devices, electrocardiogram displays, ultrasonic diagnostic devices, or endoscope displays), fish finders, one or more suitable measuring instruments, meters (for example, meters for a vehicle, an aircraft, and a vessel), projectors, and/or the like.

### Electronic Equipment

The light-emitting device may be included in one or more suitable electronic equipment.

In one or more embodiments, the electronic equipment including the light-emitting device may be at least one selected from among a flat panel display, a curved display, a computer monitor, a medical monitor, a television, a billboard, a light for indoor or outdoor lighting and/or signaling, a head-up display, a fully or partially transparent display, a flexible display, a rollable display, a foldable display, a stretchable display, a laser printer, a telephone, a mobile phone, a tablet, a phablet, a personal digital assistant (PDA), a wearable device, a laptop computer, a digital camera, a camcorder, a viewfinder, a micro display, a 3D display, a virtual or augmented-reality display, a vehicle, a video wall including multiple displays tiled together, a theater or stadium screen, a phototherapy device, and a signboard.

Because the light-emitting device has effects of an improved driving voltage, excellent or suitable luminescence efficiency, and a long lifespan, the electronic equipment including the light-emitting device may have characteristics with high luminance, high resolution, and low power consumption.

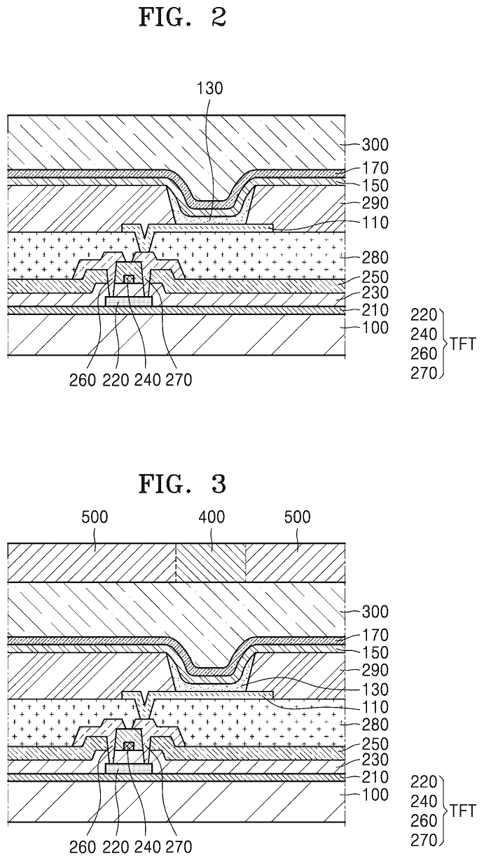

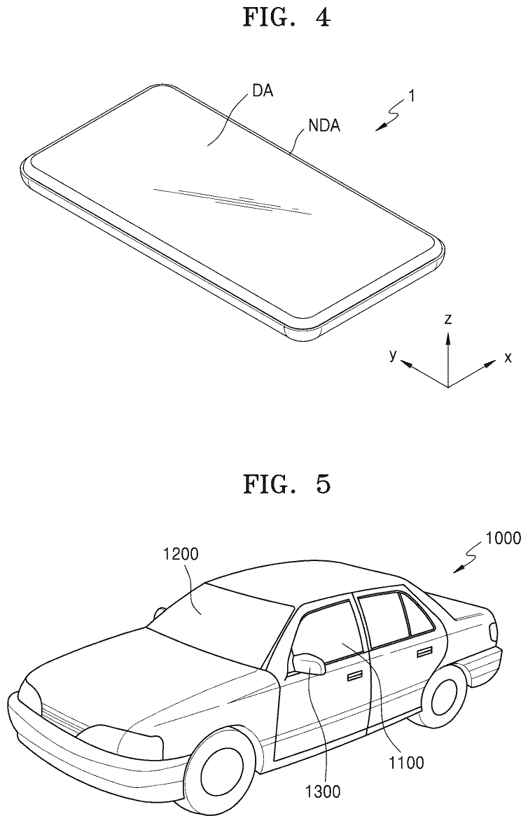

### Description of FIGS. 2 and 3

FIG. 2 is a cross-sectional view of an electronic apparatus according to one or more embodiments.

The electronic apparatus of FIG. 2 includes a substrate 100, a thin-film transistor (TFT), a light-emitting device, and an encapsulation portion 300.

The substrate 100 may be a flexible substrate, a glass substrate, or a metal substrate. A buffer layer 210 may be provided on the substrate 100. The buffer layer 210 may prevent or reduce penetration of impurities through the substrate 100 and may provide a flat surface on the substrate 100.

A TFT may be provided on the buffer layer 210. The TFT may include an activation layer 220, a gate electrode 240, a source electrode 260, and a drain electrode 270.

The activation layer 220 may include an inorganic semiconductor, such as silicon or polysilicon, an organic semiconductor, or an oxide semiconductor, and may include a source region, a drain region, and a channel region.

A gate insulating film 230 for insulating the activation layer 220 from the gate electrode 240 may be provided on the activation layer 220, and the gate electrode 240 may be provided on the gate insulating film 230.

An interlayer insulating film 250 may be provided on the gate electrode 240. The interlayer insulating film 250 may be arranged between the gate electrode 240 and the source electrode 260 and between the gate electrode 240 and the drain electrode 270, to insulate from one another.

The source electrode 260 and the drain electrode 270 may be provided on the interlayer insulating film 250. The interlayer insulating film 250 and the gate insulating film 230 may be formed to expose the source region and the drain region of the activation layer 220, and the source electrode 260 and the drain electrode 270 may be arranged to be in contact with the exposed portions of the source region and the drain region of the activation layer 220.

The TFT may be electrically connected to a light-emitting device to drive the light-emitting device, and may be covered and protected by a passivation layer 280. The passivation layer 280 may include an inorganic insulating film, an organic insulating film, or any combination thereof. A light-emitting device may be provided on the passivation layer 280. The light-emitting device includes the first electrode 110, the interlayer, and the second electrode 150.

The first electrode 110 may be provided on the passivation layer 280. The passivation layer 280 may be arranged to expose a certain region of the drain electrode 270, not fully covering the drain electrode 270, and the first electrode 110 may be arranged to be connected to the exposed region of the drain electrode 270.

A pixel-defining film 290 including an insulating material may be provided on the first electrode 110. The pixel-defining film 290 may expose a certain region of the first electrode 110, and the interlayer may be formed in the exposed region. The pixel-defining film 290 may be a polyimide-based organic film or a polyacrylic organic film. In one or more embodiments, at least some layers of the interlayer may extend beyond the upper portion of the pixel-defining film 290 to be arranged in the form of a common layer(s).

The second electrode 150 may be provided on the interlayer, and a capping layer 170 may be additionally formed on the second electrode 150. The capping layer 170 may be formed to cover the second electrode 150.

The encapsulation portion 300 may be located on the capping layer 170. The encapsulation portion 300 may be provided on a light-emitting device to protect the light-emitting device from moisture or oxygen. The encapsulation portion 300 may include: an inorganic film including silicon nitride (SiNₓ), silicon oxide (SiOₓ), indium tin oxide, indium zinc oxide, or any combination thereof; an organic film including polyethylene terephthalate, polyethylene naphthalate, polycarbonate, polyimide, polyethylene sulfonate, polyoxymethylene, polyarylate, hexamethyldisiloxane, an acrylic resin (for example, polymethyl methacrylate, polyacrylic acid, and/or the like), an epoxy-based resin (for example, aliphatic glycidyl ether (AGE), and/or the like), or any combination thereof; or a combination of the inorganic film and the organic film.

FIG. 3 is a cross-sectional view of an electronic apparatus according to another embodiment.

The electronic apparatus of FIG. 3 is the same as the electronic apparatus of FIG. 2, except that a light-shielding pattern 500 and a functional region 400 are additionally provided on the encapsulation portion 300. The functional region 400 may be i) a color filter area, ii) a color conversion area, or iii) a combination of the color filter area and the color conversion area. In one or more embodiments, the light-emitting device included in the electronic apparatus of FIG. 3 may be a tandem light-emitting device.

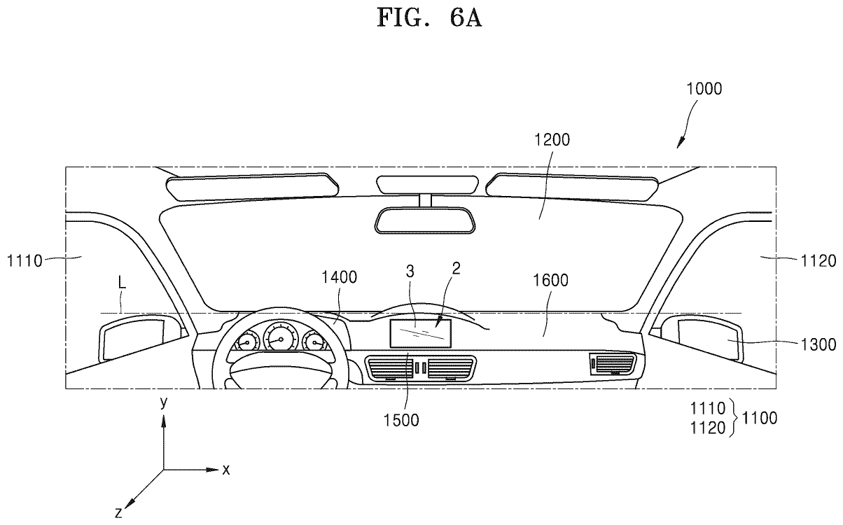

### Description of FIG. 4

FIG. 4 is a schematic perspective view of electronic equipment 1 including a light-emitting device according to one or more embodiments. The electronic equipment 1 may be, as an apparatus that displays a moving image or a still image, portable electronic equipment, such as a mobile phone, a smartphone, a tablet personal computer (PC), a mobile communication terminal, an electronic notebook, an electronic book, a portable multimedia player (PMP), a navigation, or a ultra-mobile PC (UMPC), as well as one or more suitable products, such as a television, a laptop, a monitor, a billboard, or an Internet of things (IoT). The electronic equipment 1 may be such a product or a part thereof. In some embodiments, the electronic equipment 1 may be a wearable device, such as a smart watch, a watch phone, a glasses-type or kind display, or a head mounted display (HMD), or a part of the wearable device. However, the disclosure is not limited thereto. In one or more embodiments, the electron equipment 1 may include a dashboard of a vehicle, a center fascia of a vehicle, a center information display arranged on a dashboard of a vehicle, a room mirror display replacing a side-view mirror of a vehicle, an entertainment display for the rear seat of a vehicle or a display arranged on the back of the front seat, or a head up display (HUD) installed in the front of a vehicle or projected on a front window glass, a computer generated hologram augmented reality head up display (CGH AR HUD). FIG. 4 illustrates a case in which the electronic equipment 1 is a smartphone for convenience of explanation.

The electronic equipment 1 may include a display area DA and a non-display area NDA outside the display area DA. The electronic equipment 1 may implement an image through an array of a plurality of pixels that are two-dimensionally arranged in the display area DA.

The non-display area NDA is an area that does not display an image, and may entirely surround the display area DA. On the non-display area NDA, a driver for providing electrical signals or power to display devices provided on the display area DA may be arranged. On the non-display area NDA, a pad, which is an area to which an electronic element or a printed circuit board, may be electrically connected may be arranged.

In the electronic equipment 1, the length in an x-axis direction and the length in a y-axis direction may be different from each other. In one or more embodiments, as shown in FIG. 4, the length in the x-axis direction may be less than the length in the y-axis direction. In one or more embodiments, the length in the x-axis direction may be the same as the length in the y-axis direction. In one or more embodiments, the length in the x-axis direction may be greater than the length in the y-axis direction.

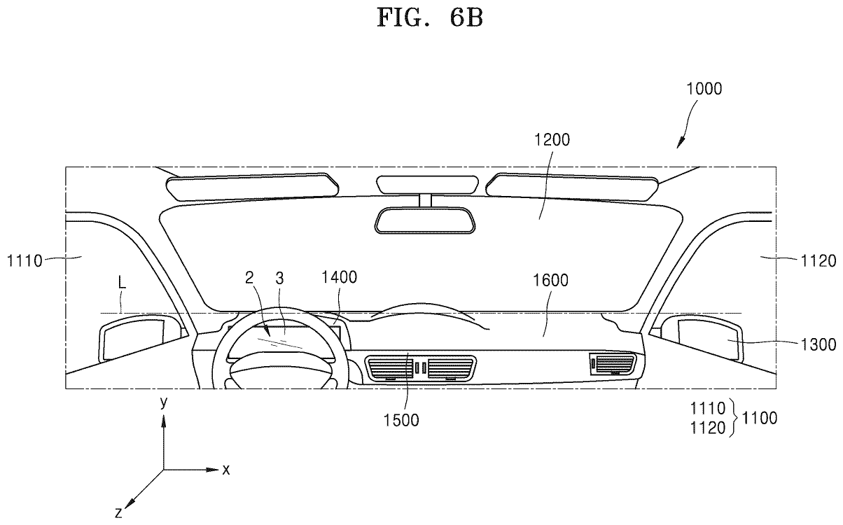

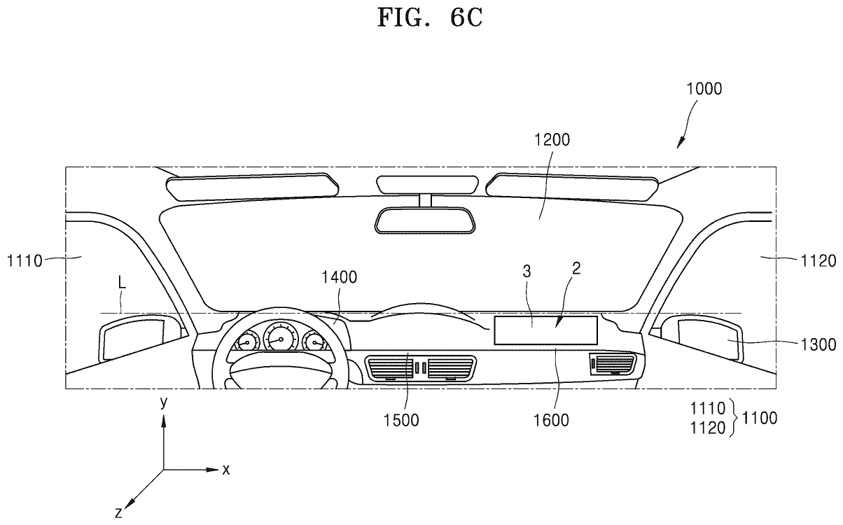

### Descriptions of FIGS. 5 and 6A to 6C

FIG. 5 is a schematic view of the exterior of a vehicle 1000 as electronic equipment including a light-emitting device according to one or more embodiments. FIGS. 6A to 6C are each a schematic view of the interior of the vehicle 1000 according to one or more embodiments.

Referring to FIGS. 5, 6A, 6B, and 6C, the vehicle 1000 may refer to one or more suitable apparatuses for moving a subject to be transported, such as a human, an object, or an animal, from a departure point to a destination point. The vehicle 1000 may include a vehicle (e.g., automobile) traveling on a road or track, a vessel moving over the sea or river, an airplane flying in the sky utilizing the action of air, and/or the like.

The vehicle 1000 may travel on a road or a track. The vehicle 1000 may move in a set, certain, or predetermined direction according to rotation of at least one wheel. In one or more embodiments, the vehicle 1000 may include a three-wheeled or four-wheeled vehicle, a construction machine, a two-wheeled vehicle, a prime mover device, a bicycle, and a train running on a track.

The vehicle 1000 may include a vehicle body having an interior and an exterior, and a chassis in which mechanical apparatuses necessary for driving are installed as other parts except for the vehicle body. The exterior of the vehicle body may include a front panel, a bonnet, a roof panel, a rear panel, a trunk, a pillar provided at a boundary between doors, and/or the like. The chassis of the vehicle 1000 may include a power generating device, a power transmitting device, a driving device, a steering device, a braking device, a suspension device, a transmission device, a fuel device, front and rear wheels, left and right wheels, and/or the like.

The vehicle 1000 may include a side window glass 1100, a front window glass 1200, a side-view mirror 1300, a cluster 1400, a center fascia 1500, a passenger seat dashboard 1600, and a display apparatus 2.

The side window glass 1100 and the front window glass 1200 may be partitioned by a fillar arranged between the side window glass 1100 and the front window glass 1200.

The side window glass 1100 may be installed on the side of the vehicle 1000. In one or more embodiments, the side window glass 1100 may be installed on a door of the vehicle 1000. A plurality of side window glasses 1100 may be provided and may face each other. In one or more embodiments, the side window glass 1100 may include a first side window glass 1110 and a second side window glass 1120. In one or more embodiments, the first side window glass 1110 may be arranged adjacent to the cluster 1400. The second side window glass 1120 may be arranged adjacent to the passenger seat dashboard 1600.

In one or more embodiments, the side window glasses 1100 may be spaced and/or apart from each other in an x-axis direction or a direction opposite to an x-axis direction. In one or more embodiments, the first side window glass 1110 and the second side window glass 1120 may be spaced and/or apart from each other in the x-axis direction or the direction opposite to an x-axis direction. In other words, an imaginary straight line L connecting the side window glasses 1100 may extend in the x-axis direction or the direction opposite to an x-axis direction. In one or more embodiments, an imaginary straight line L connecting the first side window glass 1110 and the second side window glass 1120 to each other may extend in the x-axis direction or the direction opposite to an x-axis direction.

The front window glass 1200 may be installed in front of the vehicle 1000. The front window glass 1200 may be arranged between the side window glasses 1100 facing each other.

The side-view mirror 1300 may provide a rear view of the vehicle 1000. The side-view mirror 1300 may be installed on the exterior of the vehicle body. In one or more embodiments, a plurality of side-view mirrors 1300 may be provided. Any one of the plurality of side-view mirrors 1300 may be arranged outside the first side window glass 1110. The other one of the plurality of side-view mirrors 1300 may be arranged outside the second side window glass 1120.

The cluster 1400 may be arranged in front of the steering wheel. The cluster 1400 may include a tachometer, a speedometer, a coolant thermometer, a fuel gauge turn indicator, a high beam indicator, a warning lamp, a seat belt warning lamp, an odometer, an automatic shift selector indicator lamp, a door open warning lamp, an engine oil warning lamp, and/or a low fuel warning light.

The center fascia 1500 may include a control panel on which a plurality of buttons for adjusting an audio device, an air conditioning device, and a heater of a seat are provided. The center fascia 1500 may be arranged on one side of the cluster 1400.

The passenger seat dashboard 1600 may be spaced and/or apart from the cluster 1400 with the center fascia 1500 arranged therebetween. In one or more embodiments, the cluster 1400 may be arranged to correspond to a driver seat, and the passenger seat dashboard 1600 may be arranged to correspond to a passenger seat. In one or more embodiments, the cluster 1400 may be adjacent to the first side window glass 1110, and the passenger seat dashboard 1600 may be adjacent to the second side window glass 1120.

In one or more embodiments, the display apparatus 2 may include a display panel 3, and the display panel 3 may display an image. The display apparatus 2 may be arranged inside the vehicle 1000. In one or more embodiments, the display apparatus 2 may be arranged between the side window glasses 1100 facing each other. The display apparatus 2 may be arranged on at least one selected from among the cluster 1400, the center fascia 1500, and the passenger seat dashboard 1600.

The display apparatus 2 may include an organic light-emitting display, an inorganic light-emitting display, a quantum dot display, and/or the like. Hereinafter, an organic light-emitting display apparatus including the light-emitting device according to the disclosure will be described as an example of the display apparatus 2 according to one or more embodiments, but one or more suitable types (kinds) of display apparatuses as described may be utilized in embodiments.

Referring to FIG. 6A, the display apparatus 2 may be arranged on the center fascia 1500. In one or more embodiments, the display apparatus 2 may display navigation information. In one or more embodiments, the display apparatus 2 may display audio, video, or information regarding vehicle settings.

Referring to FIG. 6B, the display apparatus 2 may be arranged on the cluster 1400. In this case, the cluster 1400 may display driving information and/or the like through the display apparatus 2. In other words, the cluster 1400 may be implemented digitally. The digital cluster 1400 may display vehicle information and driving information as images. In one or more embodiments, a needle and a gauge of a tachometer and one or more suitable warning light icons may be displayed by a digital signal.

Referring to FIG. 6C, the display apparatus 2 may be arranged on the passenger seat dashboard 1600. The display apparatus 2 may be embedded in the passenger seat dashboard 1600 or arranged on the passenger seat dashboard 1600. In one or more embodiments, the display apparatus 2 arranged on the passenger seat dashboard 1600 may display an image related to information displayed on the cluster 1400 and/or information displayed on the center fascia 1500. In one or more embodiments, the display apparatus 2 arranged on the passenger seat dashboard 1600 may display information different from information displayed on the cluster 1400 and/or information displayed on the center fascia 1500.

### Manufacturing Method

The layers included in the hole transport region 120, the emission layer 130, and the layers included in the electron transport region 140 may be formed in a certain region by utilizing one or more suitable methods such as vacuum deposition, spin coating, casting, Langmuir-Blodgett (LB) deposition, ink-jet printing, laser-printing, laser-induced thermal imaging (LITI), and/or the like.

When the layers included in the hole transport region 120, the emission layer 130, and the layers included in the electron transport region 140 are formed by vacuum deposition, the deposition may be performed at a deposition temperature of about 100° C. to about 500° C., a vacuum degree of about 10⁻⁸ torr to about 10⁻³ torr, and a deposition speed of about 0.01 Å/sec to about 100 Å/sec, depending on a material to be included in a layer to be formed and the structure of a layer to be formed.

### Definition of Terms

The term “C₃-C₆₀ carbocyclic group” as utilized herein refers to a cyclic group consisting of carbon only as a ring-forming atom and having three to sixty carbon atoms.

The term “C₁-C₆₀ heterocyclic group” as utilized herein refers to a cyclic group that has one to sixty carbon atoms and further has, in addition to carbon, a heteroatom as a ring-forming atom.

The C₃-C₆₀ carbocyclic group and the C₁-C₆₀ heterocyclic group may each be a monocyclic group consisting of one ring or a polycyclic group in which two or more rings are condensed with each other. In one or more embodiments, the number of ring-forming atoms of the C₁-C₆₀ heterocyclic group may be 3 to 61.

The “cyclic group” as utilized herein may include both (e.g., simultaneously) the C₃-C₆₀ carbocyclic group and the C₁-C₆₀ heterocyclic group.

The term “π electron-rich C₃-C₆₀ cyclic group” as utilized herein refers to a cyclic group that has three to sixty carbon atoms and does not include *—N=*′ as a ring-forming moiety.

The term “π electron-deficient nitrogen-containing C₁-C₆₀ cyclic group” as utilized herein refers to a heterocyclic group that has one to sixty carbon atoms and includes *—N=*′ as a ring-forming moiety.

In one or more embodiments
the C₃-C₆₀ carbocyclic group may be i) Group T₁ or ii) a condensed cyclic group in which two or more of Group T₁ are condensed with each other (for example, a cyclopentadiene group, an adamantane group, a norbornane group, a benzene group, a pentalene group, a naphthalene group, an azulene group, an indacene group, an acenaphthylene group, a phenalene group, a phenanthrene group, an anthracene group, a fluoranthene group, a triphenylene group, a pyrene group, a chrysene group, a perylene group, a pentaphene group, a heptalene group, a naphthacene group, a picene group, a hexacene group, a pentacene group, a rubicene group, a coronene group, an ovalene group, an indene group, a fluorene group, a spiro-bifluorene group, a benzofluorene group, an indenophenanthrene group, or an indenoanthracene group),
the C₁-C₆₀ heterocyclic group may be i) Group T₂, ii) a condensed cyclic group in which two or more of Group T₂ are condensed with each other, or iii) a condensed cyclic group in which at least one Group T₂ and at least one Group T₁ are condensed with each other (for example, a pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, a xanthene group, and/or the like),
the π electron-rich C₃-C₆₀ cyclic group may be i) Group T₁, ii) a condensed cyclic group in which two or more of Group T₁ are condensed with each other, iii) Group T₃, iv) a condensed cyclic group in which two or more of Group T₃ are condensed with each other, or v) a condensed cyclic group in which at least one Group T₃ and at least one Group T₁ are condensed with each other (for example, the C₃-C₆₀ carbocyclic group, a 1H-pyrrole group, a silole group, a borole group, a 2H-pyrrole group, a 3H-pyrrole group, a thiophene group, a furan group, an indole group, a benzoindole group, a naphthoindole group, an isoindole group, a benzoisoindole group, a naphthoisoindole group, a benzosilole group, a benzothiophene group, a benzofuran group, a carbazole group, a dibenzosilole group, a dibenzothiophene group, a dibenzofuran group, an indenocarbazole group, an indolocarbazole group, a benzofurocarbazole group, a benzothienocarbazole group, a benzosilolocarbazole group, a benzoindolocarbazole group, a benzocarbazole group, a benzonaphthofuran group, a benzonaphthothiophene group, a benzonaphthosilole group, a benzofurodibenzofuran group, a benzofurodibenzothiophene group, a benzothienodibenzothiophene group, and/or the like), and
the π electron-deficient nitrogen-containing C₁-C₆₀ cyclic group may be i) Group T₄, ii) a condensed cyclic group in which two or more of Group T₄ are condensed with each other, iii) a condensed cyclic group in which at least one Group T₄ and at least one Group T₁ are condensed with each other, iv) a condensed cyclic group in which at least one Group T₄ and at least one Group T₃ are condensed with each other, or v) a condensed cyclic group in which at least one Group T₄, at least one Group T₁, and at least one Group T₃ are condensed with one another (for example, a pyrazole group, an imidazole group, a triazole group, an oxazole group, an isoxazole group, an oxadiazole group, a thiazole group, an isothiazole group, a thiadiazole group, a benzopyrazole group, a benzimidazole group, a benzoxazole group, a benzoisoxazole group, a benzothiazole group, a benzoisothiazole group, a pyridine group, a pyrimidine group, a pyrazine group, a pyridazine group, a triazine group, a quinoline group, an isoquinoline group, a benzoquinoline group, a benzoisoquinoline group, a quinoxaline group, a benzoquinoxaline group, a quinazoline group, a benzoquinazoline group, a phenanthroline group, a cinnoline group, a phthalazine group, a naphthyridine group, an imidazopyridine group, an imidazopyrimidine group, an imidazotriazine group, an imidazopyrazine group, an imidazopyridazine group, an azacarbazole group, an azafluorene group, an azadibenzosilole group, an azadibenzothiophene group, an azadibenzofuran group, and/or the like).

Group T1 may be a cyclopropane group, a cyclobutane group, a cyclopentane group, a cyclohexane group, a cycloheptane group, a cyclooctane group, a cyclobutene group, a cyclopentene group, a cyclopentadiene group, a cyclohexene group, a cyclohexadiene group, a cycloheptene group, an adamantane group, a norbornane (or bicyclo[2.2.1]heptane) group, a norbornene group, a bicyclo[1.1.1]pentane group, a bicyclo[2.1.1]hexane group, a bicyclo[2.2.2]octane group, or a benzene group.

Group T2 may be a furan group, a thiophene group, a 1H-pyrrole group, a silole group, a borole group, a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, a tetrazine group, a pyrrolidine group, an imidazolidine group, a dihydropyrrole group, a piperidine group, a tetrahydropyridine group, a dihydropyridine group, a hexahydropyrimidine group, a tetrahydropyrimidine group, a dihydropyrimidine group, a piperazine group, a tetrahydropyrazine group, a dihydropyrazine group, a tetrahydropyridazine group, or a dihydropyridazine group.

Group T3 may be a furan group, a thiophene group, a 1H-pyrrole group, a silole group, or a borole group.

Group T4 may be a 2H-pyrrole group, a 3H-pyrrole group, an imidazole group, a pyrazole group, a triazole group, a tetrazole group, an oxazole group, an isoxazole group, an oxadiazole group, a thiazole group, an isothiazole group, a thiadiazole group, an azasilole group, an azaborole group, a pyridine group, a pyrimidine group, a pyrazine group, a pyridazine group, a triazine group, or a tetrazine group.

The terms “the cyclic group”, “the C₃-C₆₀ carbocyclic group”, “the C₁-C₆₀ heterocyclic group”, “the π electron-rich C₃-C₆₀ cyclic group” or “the π electron-deficient nitrogen-containing C₁-C₆₀ cyclic group” as used herein, refer to a monovalent or polyvalent group (for example, a divalent group, a trivalent group, a tetravalent group, or the like) that is condensed with (e.g., combined together with) a cyclic group, according to the structure of a formula for which the corresponding term is utilized.

In one or more embodiments, the “benzene group” may be a benzo group, a phenyl group, a phenylene group, and/or the like, which may be easily understood by those of ordinary skill in the art according to the structure of a formula including the “benzene group.”

Depending on context, a divalent group may refer or be a polyvalent group (e.g., trivalent, tetravalent, etc., and not just divalent) per, e.g., the structure of a formula in connection with which of the terms are utilized.

Examples of the monovalent C₃-C₆₀ carbocyclic group and monovalent C₁-C₆₀ heterocyclic group may include a C₃-C₁₀ cycloalkyl group, a C₁-C₁₀ heterocycloalkyl group, a C₃-C₁₀ cycloalkenyl group, a C₁-C₁₀ heterocycloalkenyl group, a C₆-C₆₀ aryl group, a C₁-C₆₀ heteroaryl group, a monovalent non-aromatic condensed polycyclic group, and a monovalent non-aromatic condensed heteropolycyclic group.

Examples of the divalent C₃-C₆a carbocyclic group and the divalent C₁-C₁₀ heterocyclic group may include a C₃-C₁₀ cycloalkylene group, a C₁-C₁₀ heterocycloalkylene group, a C₃-C₁₀ cycloalkenylene group, a C₁-C₁₀ heterocycloalkenylene group, a C₆-C₆₀ arylene group, a C₁-C₆₀ heteroarylene group, a divalent non-aromatic condensed polycyclic group, and a divalent non-aromatic condensed heteropolycyclic group.

The term “C₁-C₆₀ alkyl group” as utilized herein refers to a linear or branched aliphatic hydrocarbon monovalent group that has one to sixty carbon atoms, and examples thereof include a methyl group, an ethyl group, an n-propyl group, an isopropyl group, an n-butyl group, a sec-butyl group, an isobutyl group, a tert-butyl group, an n-pentyl group, a tert-pentyl group, a neopentyl group, an isopentyl group, a sec-pentyl group, a 3-pentyl group, a sec-isopentyl group, an n-hexyl group, an isohexyl group, a sec-hexyl group, a tert-hexyl group, an n-heptyl group, an isoheptyl group, a sec-heptyl group, a tert-heptyl group, an n-octyl group, an isooctyl group, a sec-octyl group, a tert-octyl group, an n-nonyl group, an isononyl group, a sec-nonyl group, a tert-nonyl group, an n-decyl group, an isodecyl group, a sec-decyl group, and a tert-decyl group.

The term “C₁-C₆₀ alkylene group” as utilized herein refers to a divalent group having the same structure as the C₁-C₆₀ alkyl group.

The term “C₂-C₆₀ alkenyl group” as utilized herein refers to a monovalent hydrocarbon group having at least one carbon-carbon double bond in the middle or at the terminus of the C₂-C₆₀ alkyl group, and examples thereof include an ethenyl group, a propenyl group, and a butenyl group.

The term “C₂-C₆₀ alkenylene group” as utilized herein refers to a divalent group having the same structure as the C₂-C₆₀ alkenyl group.

The term “C₂-C₆₀ alkynyl group” as utilized herein refers to a monovalent hydrocarbon group having at least one carbon-carbon triple bond in the middle or at the terminus of the C₂-C₆₀ alkyl group, and examples thereof include an ethynyl group and a propynyl group.

The term “C₂-C₆₀ alkynylene group” as utilized herein refers to a divalent group having the same structure as the C₂-C₆₀ alkynyl group.

The term “C₁-C₆₀ alkoxy group” as utilized herein refers to a monovalent group represented by —OA₁₀₁ (wherein A₁₀₁ is the C₁-C₆₀ alkyl group), and examples thereof include a methoxy group, an ethoxy group, and an isopropyloxy group.

The term “C₃-C₁₀ cycloalkyl group” as utilized herein refers to a monovalent saturated hydrocarbon cyclic group having three to ten carbon atoms, and examples thereof include a cyclopropyl group, a cyclobutyl group, a cyclopentyl group, a cyclohexyl group, a cycloheptyl group, a cyclooctyl group, an adamantanyl group, a norbornanyl group (or bicyclo[2.2.1]heptyl group), a bicyclo[1.1.1]pentyl group, a bicyclo[2.1.1]hexyl group, and a bicyclo[2.2.2]octyl group.

The term “C₃-C₁₀ cycloalkylene group” as utilized herein refers to a divalent group having the same structure as the C₃-C₁₀ cycloalkyl group.

The term “C₁-C₁₀ heterocycloalkyl group” as utilized herein refers to a monovalent cyclic group that has one to ten carbon atoms and further includes, in addition to the carbon atoms, at least one heteroatom as a ring-forming atom, and examples thereof include a 1,2,3,4-oxatriazolidinyl group, a tetrahydrofuranyl group, and a tetrahydrothiophenyl group.

The term “C₁-C₁₀ heterocycloalkylene group” as utilized herein refers to a divalent group having the same structure as the C₁-C₁₀ heterocycloalkyl group.

The term “C₃-C₁₀ cycloalkenyl group” as utilized herein refers to a monovalent cyclic group that has three to ten carbon atoms and at least one carbon-carbon double bond in the ring thereof and no aromaticity, and examples thereof include a cyclopentenyl group, a cyclohexenyl group, and a cycloheptenyl group.

The term “C₃-C₁₀ cycloalkenylene group” as utilized herein refers to a divalent group having the same structure as the C₃-C₁₀ cycloalkenyl group.

The term “C₁-C₁₀ heterocycloalkenyl group” as utilized herein refers to a monovalent cyclic group that has one to ten carbon atoms, further includes, in addition to the carbon atoms, at least one heteroatom as a ring-forming atom, and has at least one double bond in the ring thereof. Examples of the C₁-C₁₀ heterocycloalkenyl group include a 4,5-dihydro-1,2,3,4-oxatriazolyl group, a 2,3-dihydrofuranyl group, and a 2,3-dihydrothiophenyl group.

The term “C₁-C₁₀ heterocycloalkenylene group” as utilized herein refers to a divalent group having the same structure as the C₁-C₁₀ heterocycloalkenyl group.

The term “C₆-C₆₀ aryl group” as utilized herein refers to a monovalent group having a carbocyclic aromatic system of six to sixty carbon atoms.

The term “C₆-C₆₀ arylene group” as utilized herein refers to a divalent group having a carbocyclic aromatic system of six to sixty carbon atoms.

Examples of the C₆-C₆₀ aryl group include a phenyl group, a pentalenyl group, a naphthyl group, an azulenyl group, an indacenyl group, an acenaphthyl group, a phenalenyl group, a phenanthrenyl group, an anthracenyl group, a fluoranthenyl group, a triphenylenyl group, a pyrenyl group, a chrysenyl group, a perylenyl group, a pentaphenyl group, a heptalenyl group, a naphthacenyl group, a picenyl group, a hexacenyl group, a pentacenyl group, a rubicenyl group, a coronenyl group, and an ovalenyl group.

When the C₆-C₆₀ aryl group and the C₆-C₆₀ arylene group each include two or more rings, the two or more rings may be condensed with each other.

The term “C₁-C₆₀ heteroaryl group” as utilized herein refers to a monovalent group having a heterocyclic aromatic system of one to sixty carbon atoms, further including, in addition to carbon atoms, at least one heteroatom, as ring-forming atoms.

The term “C₁-C₆₀ heteroarylene group” as utilized herein refers to a divalent group having a heterocyclic aromatic system of one to sixty carbon atoms, further including, in addition to carbon atoms, at least one heteroatom, as ring-forming atoms.

Examples of the C₁-C₆₀ heteroaryl group include a pyridinyl group, a pyrimidinyl group, a pyrazinyl group, a pyridazinyl group, a triazinyl group, a quinolinyl group, a benzoquinolinyl group, an isoquinolinyl group, a benzoisoquinolinyl group, a quinoxalinyl group, a benzoquinoxalinyl group, a quinazolinyl group, a benzoquinazolinyl group, a cinnolinyl group, a phenanthrolinyl group, a phthalazinyl group, and a naphthyridinyl group.

When the C₁-C₆₀ heteroaryl group and the C₁-C₆₀ heteroarylene group each include two or more rings, the two or more rings may be condensed with each other.

The term “monovalent non-aromatic condensed polycyclic group” as utilized herein refers to a monovalent group having two or more rings condensed with each other, only carbon atoms (for example, eight to sixty carbon atoms) as ring-forming atoms, and no aromaticity in its molecular structure when considered as a whole. Examples of the monovalent non-aromatic condensed polycyclic group include an indenyl group, a fluorenyl group, a spiro-bifluorenyl group, a benzofluorenyl group, an indenophenanthrenyl group, and an indeno anthracenyl group.

The term “divalent non-aromatic condensed polycyclic group” as utilized herein refers to a divalent group having the same structure as the monovalent non-aromatic condensed polycyclic group.

The term “monovalent non-aromatic condensed heteropolycyclic group” as utilized herein refers to a monovalent group that has two or more rings condensed with each other, further includes, in addition to carbon atoms (for example, one to sixty carbon atoms), at least one heteroatom as a ring-forming atom, and has no aromaticity in its molecular structure when considered as a whole. Examples of the monovalent non-aromatic condensed heteropolycyclic group may include a pyrrolyl group, a thiophenyl group, a furanyl group, an indolyl group, a benzoindolyl group, a naphthoindolyl group, an isoindolyl group, a benzoisoindolyl group, a naphthoisoindolyl group, a benzosilolyl group, a benzothiophenyl group, a benzofuranyl group, a carbazolyl group, a dibenzosilolyl group, a dibenzothiophenyl group, a dibenzofuranyl group, an azacarbazolyl group, an azafluorenyl group, an azadibenzosilolyl group, an azadibenzothiophenyl group, an azadibenzofuranyl group, a pyrazolyl group, an imidazolyl group, a triazolyl group, a tetrazolyl group, an oxazolyl group, an isoxazolyl group, a thiazolyl group, an isothiazolyl group, an oxadiazolyl group, a thiadiazolyl group, a benzopyrazolyl group, a benzimidazolyl group, a benzoxazolyl group, a benzothiazolyl group, a benzoxadiazolyl group, a benzothiadiazolyl group, an imidazopyridinyl group, an imidazopyrimidinyl group, an imidazotriazinyl group, an imidazopyrazinyl group, an imidazopyridazinyl group, an indeno carbazolyl group, an indolocarbazolyl group, a benzofurocarbazolyl group, a benzothienocarbazolyl group, a benzosilolocarbazolyl group, a benzoindolocarbazolyl group, a benzocarbazolyl group, a benzonaphthofuranyl group, a benzonaphthothiophenyl group, a benzonaphthosilolyl group, a benzofurodibenzofuranyl group, a benzofurodibenzothiophenyl group, and a benzothienodibenzothiophenyl group.

The term “divalent non-aromatic condensed heteropolycyclic group” as utilized herein refers to a divalent group having the same structure as the monovalent non-aromatic condensed heteropolycyclic group.

The term “C₆-C₆₀ aryloxy group” as utilized herein indicates —OA₁₀₂ (wherein A₁₀₂ is the C₆-C₆₀ aryl group).

The term “C₆-C₆₀ arylthio group” as utilized herein indicates —SA₁₀₃ (wherein A₁₀₃ is the C₆-C₆₀ aryl group).

The term “C₇-C₆₀ arylalkyl group” as utilized herein refers to -A₁₀₄A₁₀₅ (wherein A₁₀₄ is a C₁-C₅₄ alkylene group, and A₁₀₅ is a C₆-C₅₉ aryl group).

The term “C₂-C₆₀ heteroarylalkyl group” as utilized herein refers to -A₁₀₆A₁₀₇ (wherein A₁₀₆ is a C₁-C₅₉ alkylene group, and A₁₀₇ is a C₁-C₅₉ heteroaryl group), and the term “C₁-C₆₀ alkylthio group” as used herein indicates —SA₁₀₈ (wherein A₁₀₈ is the C₁-C₆₀ alkyl group).

The term “R₁₀ₐ” as utilized herein may be:
deuterium, —F, —Cl, —Br, —I, a hydroxyl group, a cyano group, or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or any combination thereof;
a C₃-C₁₀ carbocyclic group, a C₁-C₆₀ heterocyclic group, a C₆-C₆₀ aryloxy group, a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or any combination thereof; or
—Si(Q₃₁)(Q₃₂)(Q₃₃), —N(Q₃₁)(Q₃₂), —B(Q₃₁)(Q₃₂), —C(═O)(Q₃₁), —S(═O)₂(Q₃₁), or —P(═O)(Q₃₁)(Q₃₂).

Herein, Q₁ to Q₃, Q₁₁ to Q₁₃, Q₂₁ to Q₂₃, and Q₃₁ to Q₃₃ may each independently be: hydrogen; deuterium; —F; —C₁; —Br; —I; a hydroxyl group; a cyano group; a nitro group; or a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or any combination thereof.

The term “heteroatom” as utilized herein refers to any atom other than a carbon atom. Examples of the heteroatom include O, S, N, P, Si, B, Ge, Se, or any combination thereof.

The term “third-row transition metal” as utilized herein includes hafnium (Hf), tantalum (Ta), tungsten (W), rhenium (Re), osmium (Os), iridium (Ir), platinum (Pt), and gold (Au).

Herein, “D” may refer to deuterium, “Ph” may refer to a phenyl group, “Me” may refer to a methyl group, “Et” may refer to an ethyl group, “tert-Bu”, “tBu”, or “But” may each refer to a tert-butyl group, and “OMe” may refer to a methoxy group.

The term “biphenyl group” as utilized herein refers to “a phenyl group that is substituted with a phenyl group.” In other words, the “biphenyl group” may be a substituted phenyl group having a C₆-C₆₀ aryl group as a substituent.

The term “terphenyl group” as utilized herein refers to “a phenyl group substituted with a biphenyl group.” The “terphenyl group” may belong to i) “a substituted phenyl group” which is “a C₆-C₆₀ aryl group in which a substituent is substituted with a C₆-C₆₀ aryl group”, or ii) “a substituted phenyl group” having two substituents, each of which is “a C₆-C₆₀ aryl group.”

* and *′ as utilized herein, unless defined otherwise, each refer to a binding site to a neighboring atom in a corresponding formula or moiety.

Terms such as “substantially,” “about,” and “approximately” are used as relative terms and not as terms of degree, and are intended to account for the inherent deviations in measured or calculated values that would be recognized by those of ordinary skill in the art. They may be inclusive of the stated value and an acceptable range of deviation as determined by one of ordinary skill in the art, considering the limitations and error associated with measurement of that quantity. For example, “about” may refer to one or more standard deviations, or ±30%, 20%, 10%, 5% of the stated value.

Numerical ranges disclosed herein include and are intended to disclose all subsumed sub-ranges of the same numerical precision. For example, a range of “1.0 to 10.0” includes all subranges having a minimum value equal to or greater than 1.0 and a maximum value equal to or less than 10.0, such as, for example, 2.4 to 7.6. Applicant therefore reserves the right to amend this specification, including the claims, to expressly recite any sub-range subsumed within the ranges expressly recited herein.

The light emitting device and/or any other relevant devices or components according to embodiments of the present disclosure described herein may be implemented utilizing any suitable hardware, firmware (e.g., an application-specific integrated circuit), software, or a combination of software, firmware, and hardware. For example, the various components of the light emitting device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the embodiments of the present disclosure.

In the present disclosure, when dots or particles are spherical, “diameter” indicates an average particle diameter, and when the dots or particles are non-spherical, the “diameter” indicates a major axis length. The diameter (or size) of the particles may be measured by particle size analysis, dynamic light scattering, scanning electron microscopy, and/or transmission electron microscope photography. When the size of the particles is measured utilizing a particle size analyzer, the average particle diameter (or size) may be referred to as D50. The term “D50” as utilized herein refers to the average diameter (or size) of particles whose cumulative volume corresponds to 50 vol % in the particle size distribution (e.g., cumulative distribution), and refers to the value of the particle size corresponding to 50% from the smallest particle when the total number of particles is 100% in the distribution curve accumulated in the order of the smallest particle size to the largest particle size. Particle size analysis may be performed with a HORIBA LA-950 laser particle size analyzer.

Herein, the x-axis, y-axis, and z-axis are not limited to three axes in an orthogonal coordinate system, and may be interpreted in a broad sense including these axes. For example, the x-axis, y-axis, and z-axis may refer to those orthogonal to each other, or may refer to those in different directions that are not orthogonal to each other.

Hereinafter, compounds according to embodiments and light-emitting devices according to embodiments will be described in more detail with reference to the following synthesis examples and examples. The wording “B was utilized instead of A” utilized in describing Synthesis Examples refers to that an identical molar equivalent of B was utilized in place of A.

### EXAMPLES

### Synthesis Example 1-1 (Synthesis of Compound HD1)

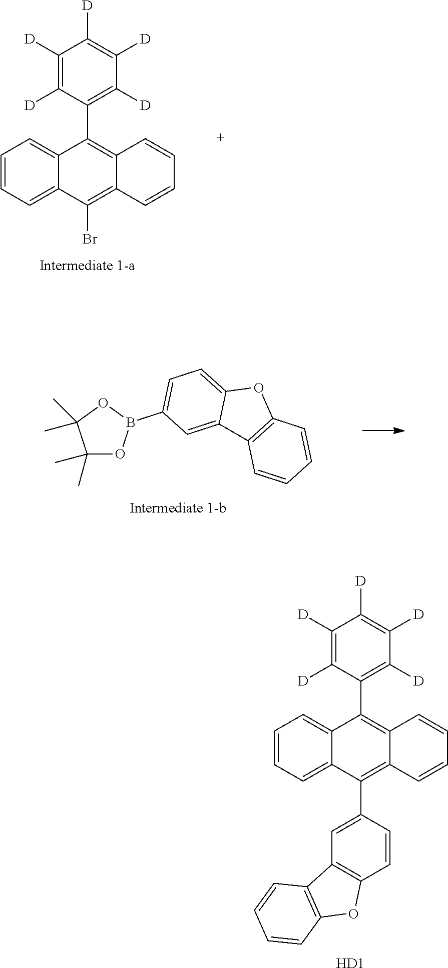

Intermediate 1-a (3.4 g, 10 mmol), Intermediate 1-b (2.9 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD1 (3.0 g, 70%).

### Synthesis Example 1-2 (Synthesis of Compound HD2)

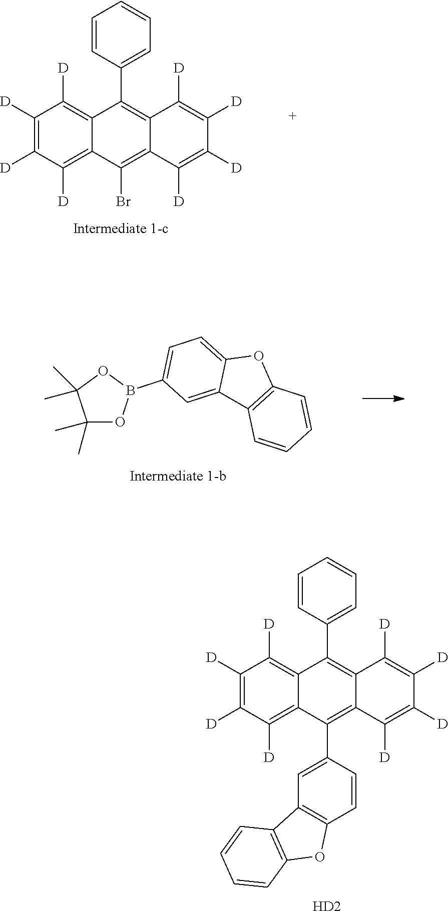

Intermediate 1-c (3.4 g, 10 mmol), Intermediate 1-b (2.9 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD2 (2.5 g, 58%).

### Synthesis Example 1-3 (Synthesis of Compound HD3)

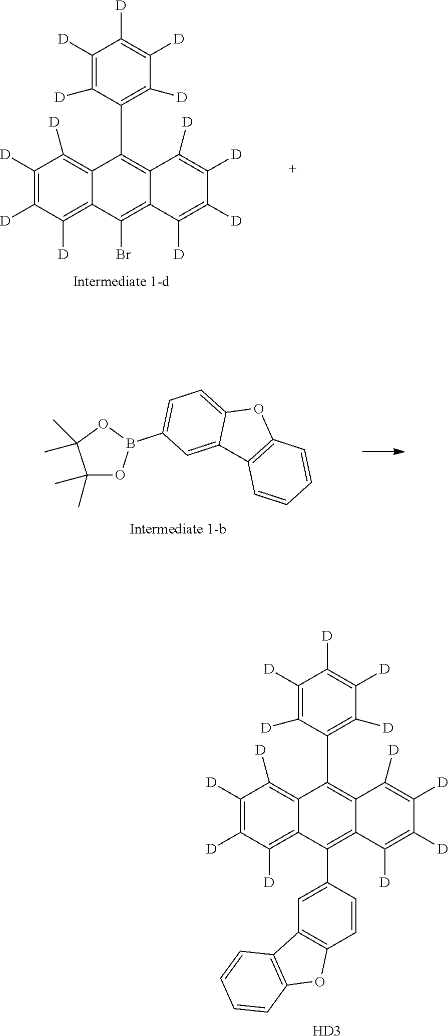

Intermediate 1-d (3.5 g, 10 mmol), Intermediate 1-b (2.9 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD3 (2.3 g, 52%).

### Synthesis Example 1-4 (Synthesis of Compound HD4)

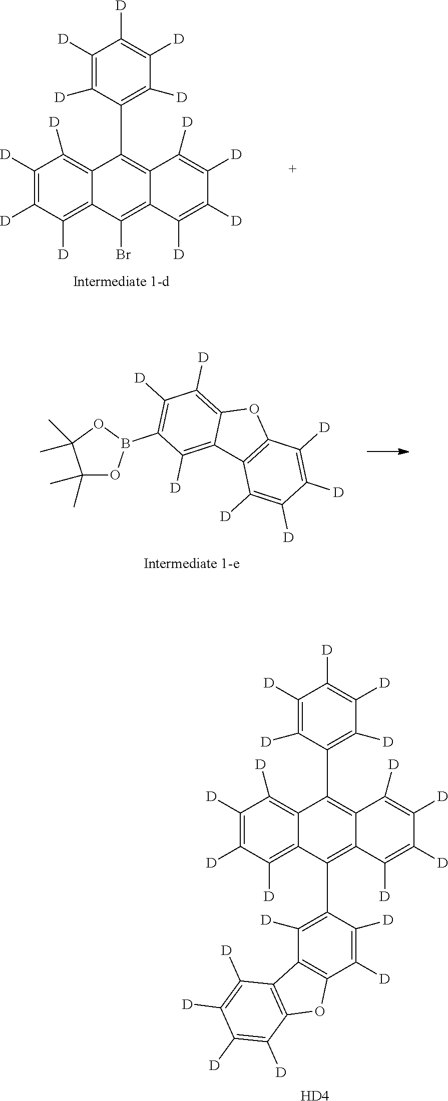

Intermediate 1-d (3.5 g, 10 mmol), Intermediate 1-e (3.0 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD4 (2.0 g, 44%).

### Synthesis Example 2-1 (Synthesis of Compound HD30)

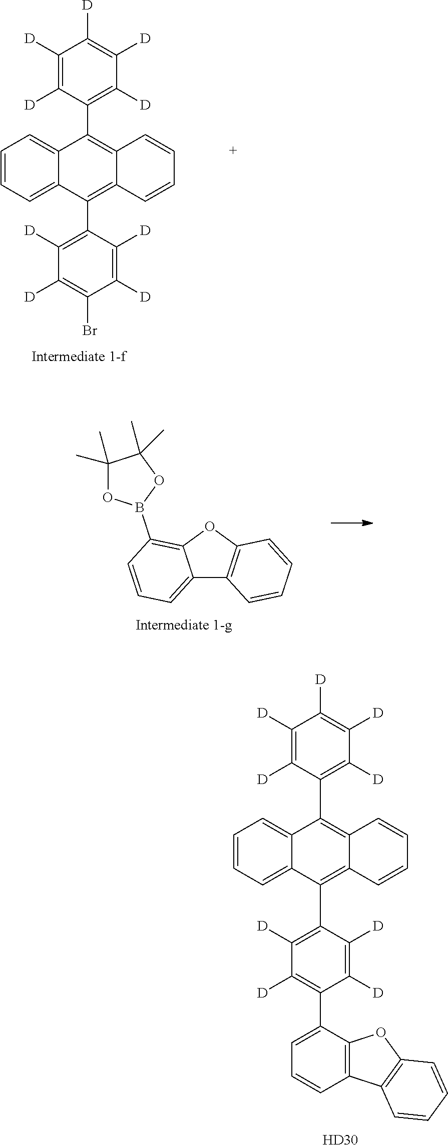

Intermediate 1-f (4.2 g, 10 mmol), Intermediate 1-g (2.9 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD30 (3.0 g, 59%).

### Synthesis Example 2-2 (Synthesis of Compound HD31)

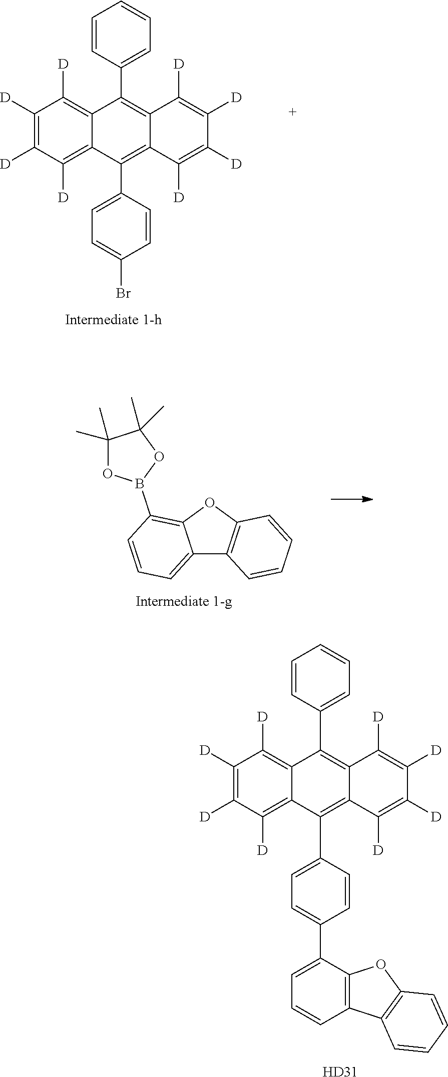

Intermediate 1-h (4.2 g, 10 mmol), Intermediate 1-g (2.9 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD31 (2.9 g, 59%).

### Synthesis Example 2-3 (Synthesis of Compound HD33)

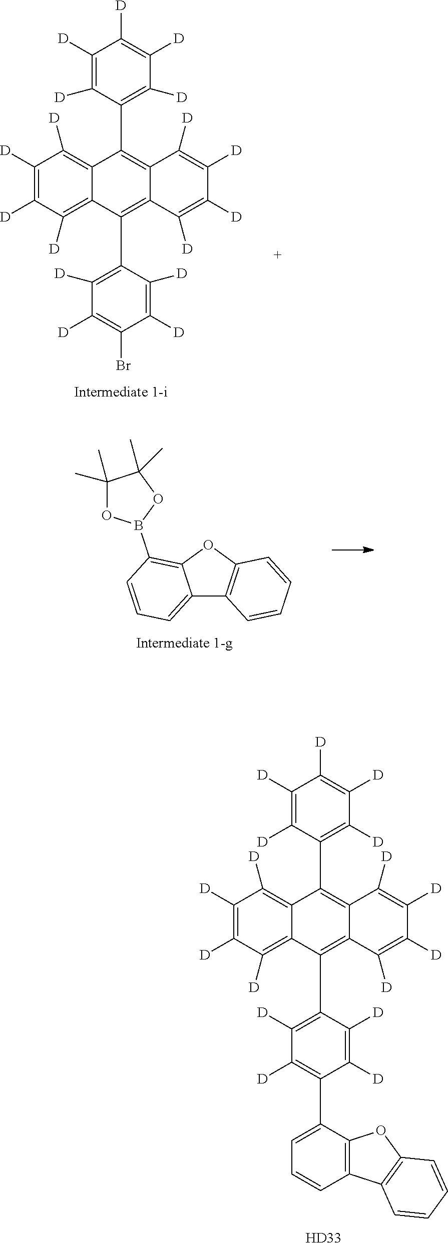

Intermediate 1-i (4.3 g, 10 mmol), Intermediate 1-g (2.9 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD33 (2.2 g, 43%).

### Synthesis Example 2-4 (Synthesis of Compound HD35)

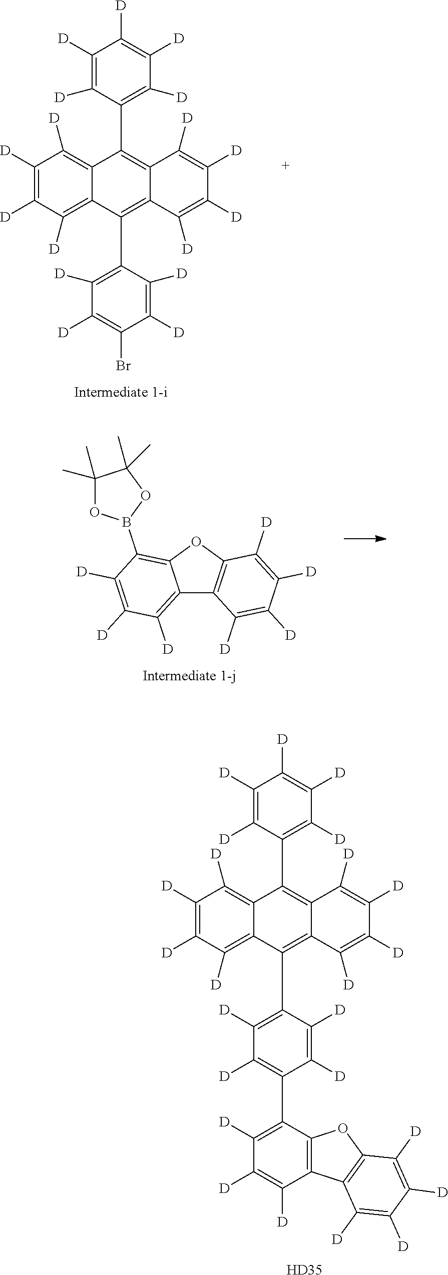

Intermediate 1-i (4.3 g, 10 mmol), Intermediate 1-j (3.0 g, 10 mmol), Pd(PPh₃)₄ (0.46 g, 3 mmol), and K₂CO₃ (4.2 g, 30 mmol) were put into a 250 mL round-bottom flask reactor, and 30 mL of toluene, 30 mL of 1,4-dioxane, and 10 mL of H₂O were put thereinto. The temperature of the reactor was raised to 90° C., and stirring was made overnight. After the reaction was completed, the temperature of the reactor was lowered to room temperature, an extraction process was performed by utilizing ethyl acetate, and an organic layer was separated. After concentration under reduced pressure, the organic layer was separated by column chromatography. Recrystallization was made from toluene and acetone to obtain Compound HD35 (2.3 g, 44%).

### Synthesis Example 3-1 (Synthesis of Compound EB1)

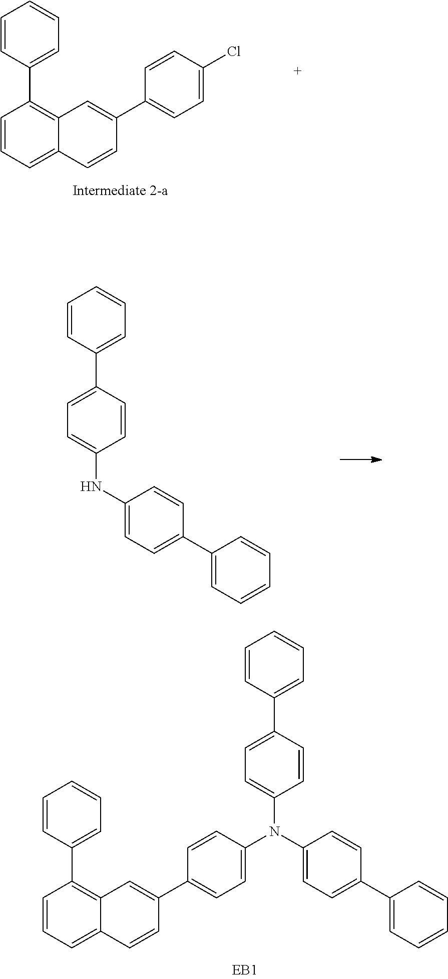

Under Ar atmosphere, Intermediate 2-a (3.1 g, 10 mmol), Pd(dba)₂ (0.22 g, 0.3 mmol), NaOtBu (1.9 g, 20 mmol), 100 mL of toluene, bis(4-biphenyl)amine (3.2 g, 10 mmol), and ^{t}Bu₃P (0.20 g, 1.0 mmol) were added to a 300 mL three-neck flask in the stated order, the temperature of the reactor was raised to 90° C., and stirring was made overnight. After the reaction was completed, the temperature of the reactor was lowered to room temperature, and water was added to the reaction solvent to separate an organic layer. Toluene was added to the water layer to further extract an organic layer, and the organic layers were combined, washed with distilled water, and then dried over MgSO₄. After concentration under reduced pressure, separation was made by column chromatography by utilizing a mixed solvent of hexane and toluene to obtain Compound EB1 (4.8 g, 80%).

### Synthesis Example 3-2 (Synthesis of Compound EB41)

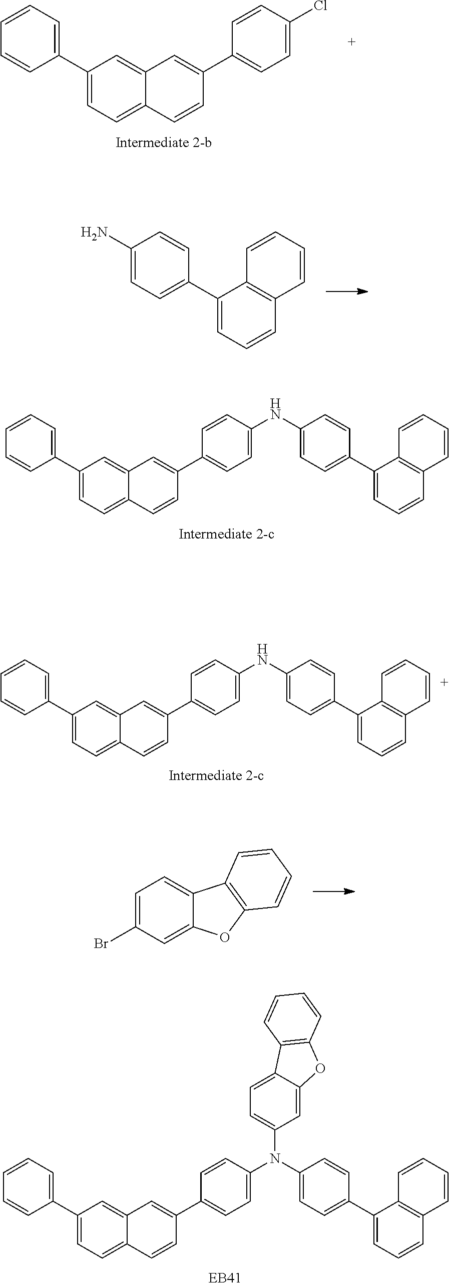 [SMILES: Clc1ccc(-c2ccc3ccc(-c4ccccc4)cc3c2)cc1 ; Nc1ccc(-c2cccc3ccccc23)cc1 ; c1ccc(-c2ccc3ccc(-c4ccc(Nc5ccc(-c6cccc7ccccc67)cc5)cc4)cc3c2)cc1 ; c1ccc(-c2ccc3ccc(-c4ccc(Nc5ccc(-c6cccc7ccccc67)cc5)cc4)cc3c2)cc1 ; Brc1ccc2c(c1)oc1ccccc12 ; c1ccc(-c2ccc3ccc(-c4ccc(N(c5ccc(-c6cccc7ccccc67)cc5)c5ccc6c(c5)oc5ccccc56)cc4)cc3c2)cc1]

### Synthesis of Intermediate 2-c

Under Ar atmosphere, Intermediate 2-b (3.1 g, 10 mmol), Pd(dba)₂ (0.22 g, 0.3 mmol), NaOtBu (1.9 g, 20 mmol), 100 mL of toluene, 4-(naphthalen-1-yl)aniline (2.2 g, 10 mmol), and ¹Bu₃P (0.20 g, 1.0 mmol) were added to a 300 mL three-neck flask in the stated order, the temperature of the reactor was raised to 90° C., and stirring was made overnight. After the reaction was completed, the temperature of the reactor was lowered to room temperature, and water was added to the reaction solvent to separate an organic layer. Toluene was added to the water layer to further extract an organic layer, and the organic layers were combined, washed with distilled water, and then dried over MgSO₄. After concentration under reduced pressure, separation was made by column chromatography by utilizing a mixed solvent of hexane and toluene to obtain Intermediate 2-c (3.8 g, 75%).

### Synthesis of Compound EB1

Under Ar atmosphere, Intermediate 2-c (5.0 g, 10 mmol), Pd(dba)₂ (0.22 g, 0.3 mmol), NaOtBu (1.9 g, 20 mmol), 100 mL of toluene, 3-bromo-dibenzofuran (2.5 g, 10 mmol), and 0.39 g (0.1 equiv, 1.9 mmol) of ¹Bu₃P were added to 300 mL three-neck flask in the stated order, the temperature of the reactor was raised to 90° C., and stirring was made overnight. After the reaction was completed, the temperature of the reactor was lowered to room temperature, and water was added to the reaction solvent to separate an organic layer. The organic layer was washed with distilled water and then dried over MgSO₄. After concentration under reduced pressure, separation was made by column chromatography by utilizing a mixed solvent of hexane and toluene to obtain Compound EB41 (4.0 g, 60%).

### Synthesis Example 4-3 (Synthesis of Compound EB42)

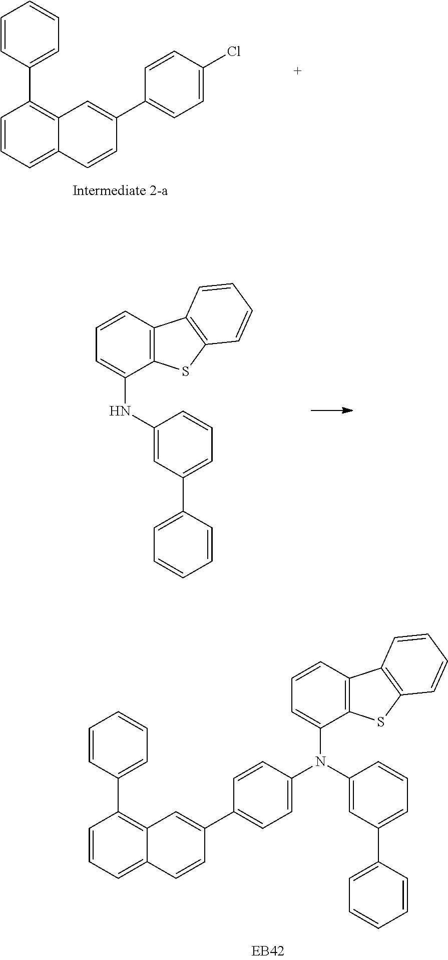 [SMILES: Clc1ccc(-c2ccc3cccc(-c4ccccc4)c3c2)cc1 ; c1ccc(-c2cccc(Nc3cccc4c3sc3ccccc34)c2)cc1 ; c1ccc(-c2cccc(N(c3ccc(-c4ccc5cccc(-c6ccccc6)c5c4)cc3)c3cccc4c3sc3ccccc34)c2)cc1]

Under Ar atmosphere, Intermediate 2-a (3.1 g, 10 mmol), Pd(dba)₂ (0.22 g, 0.3 mmol), NaOtBu (1.9 g, 20 mmol), 100 mL of toluene, N-([1,1′-biphenyl]-3-yl)dibenzothiophen-4-amine (3.5 g, 10 mmol), and ¹Bu₃P (0.20 g, 1.0 mmol) were added to a 300 mL three-neck flask in the stated order, the temperature of the reactor was raised to 90° C., and stirring was made overnight. After the reaction was completed, the temperature of the reactor was lowered to room temperature, and water was added to the reaction solvent to separate an organic layer. Toluene was added to the aqueous layer to further extract an organic layer, and the organic layers were combined, washed with distilled water, and then dried over MgSO₄. After concentration under reduced pressure, separation was made by column chromatography by utilizing a mixed solvent of hexane and toluene to obtain Compound EB42 (3.9 g, 62%).

### Synthesis Example 4-4 (Synthesis of Compound EB43)

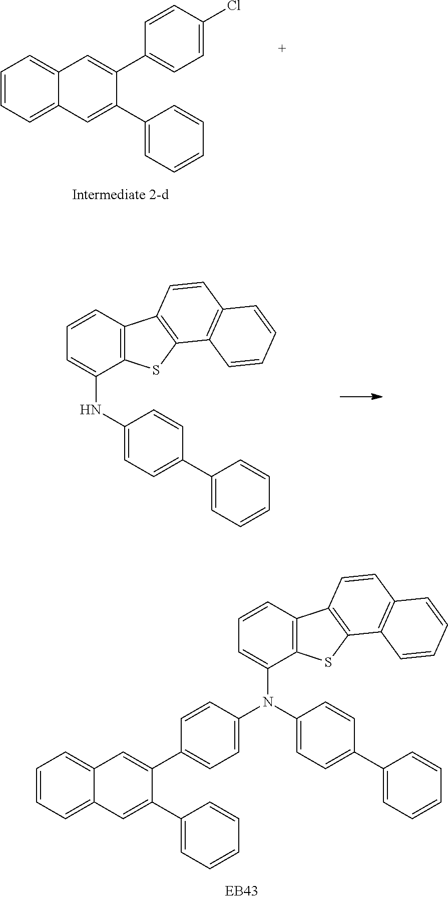 [SMILES: Clc1ccc(-c2cc3ccccc3cc2-c2ccccc2)cc1 ; c1ccc(-c2ccc(Nc3cccc4c3sc3c5ccccc5ccc43)cc2)cc1 ; c1ccc(-c2ccc(N(c3ccc(-c4cc5ccccc5cc4-c4ccccc4)cc3)c3cccc4c3sc3c5ccccc5ccc43)cc2)cc1]

Under Ar atmosphere, Intermediate 2-d (3.1 g, 10 mmol), Pd(dba)₂ (0.22 g, 0.3 mmol), NaOtBu (1.9 g, 20 mmol), 150 mL of toluene, N-([1,1′-biphenyl]-4-yl)benzo[b]naphtho[2,1-d]thiophen-10-amine (4.0 g, 10 mmol), and tBu₃P (0.20 g, 1.0 mmol) were added to a 300 mL three-neck flask in the stated order, the temperature of the reactor was raised to 90° C., and stirring was made overnight. After the reaction was completed, the temperature of the reactor was lowered to room temperature, and water was added to the reaction solvent to separate an organic layer. Toluene was added to the aqueous layer to further extract an organic layer, and the organic layers were combined, washed with distilled water, and then dried over MgSO₄. After concentration under reduced pressure, separation was made by column chromatography by utilizing a mixed solvent of hexane and toluene to obtain Compound EB43 (3.1 g, 45%).

### Comparative Example 1

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound CE1 was vacuum-deposited on the hole transport layer to form an electron blocking layer having a thickness of 50 Å.

Compound HH1 as a host and Compound D1 as a dopant were co-vacuum-deposited on the electron blocking layer to form an emission layer having a thickness of 200 Å. A weight ratio of host:dopant was 99:1.

T2T was vacuum-deposited on the emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Comparative Examples 2 to 5

Light-emitting devices were manufactured in substantially the same manner as in Comparative Example 1, except that, in forming the electron blocking layer or the emission layer, a material for the electron blocking layer and/or the host were changed to those shown in Table 1.

### Comparative Example 6

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound EB1 was vacuum-deposited on the hole transport layer to form an electron blocking layer having a thickness of 50 Å.

Compounds HH1 and HH6 as a host and Compound D1 as a dopant were co-vacuum-deposited on the electron blocking layer to form an emission layer having a thickness of 200 Å. A weight ratio of Compound HH1:Compound HH6 was the same as shown in Table 1, and a weight ratio of host:dopant was 99:1.

T2T was vacuum-deposited on the emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Comparative Examples 7 to 9

Light-emitting devices were manufactured in substantially the same manner as in Comparative Example 6, except that, in forming the electron blocking layer, Compound EB1 was changed to that shown in Tables 3 to 5.

### Examples 1 to 6

Light-emitting devices were manufactured in substantially the same manner as in Comparative Example 6, except that, in forming the emission layer, the type or kind of the mixed host and the weight ratio of the mixed host were changed to those shown in Table 2.

### Example 7

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound EB1 was vacuum-deposited on the hole transport layer to form an electron blocking layer having a thickness of 50 Å.

Compound HD4 as a host and Compound D1 as a dopant were co-vacuum-deposited on the electron blocking layer to form a first emission layer having a thickness of 100 Å. A weight ratio of host:dopant in the first emission layer was 99:1.

Compound HD35 as a host and Compound D1 as a dopant were co-vacuum-deposited on the first emission layer to form a second emission layer having a thickness of 100 Å. A weight ratio of host:dopant in the second emission layer was 99:1.

T2T was vacuum-deposited on the second emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Examples 8 to 13

Light-emitting devices were manufactured in substantially the same manner as in Comparative Example 7, except that, in (each) forming (of) the emission layer, the type or kind of the mixed host and the weight ratio of the mixed host were changed to those shown in Table 3.

### Example 14

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound EB41 was vacuum-deposited on the hole transport layer to form an electron blocking layer having a thickness of 50 Å.

Compound HD4 as a host and Compound D1 as a dopant were co-vacuum-deposited on the electron blocking layer to form a first emission layer having a thickness of 100 Å. A weight ratio of host:dopant in the first emission layer was 99:1.

Compound HD35 as a host and Compound D1 as a dopant were co-vacuum-deposited on the first emission layer to form a second emission layer having a thickness of 100 Å. A weight ratio of host:dopant in the second emission layer was 99:1.

T2T was vacuum-deposited on the second emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Examples 15 to 20

Light-emitting devices were manufactured in substantially the same manner as in Comparative Example 8, except that, in forming the emission layer, the type or kind of the mixed host and the weight ratio of the mixed host were changed to those shown in Table 4.

### Example 21

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound EB42 was vacuum-deposited on the hole transport layer to form an electron blocking layer having a thickness of 50 Å.

Compound HD4 as a host and Compound D1 as a dopant were co-vacuum-deposited on the electron blocking layer to form a first emission layer having a thickness of 100 Å. A weight ratio of host:dopant in the first emission layer was 99:1.

Compound HD35 as a host and Compound D1 as a dopant were co-vacuum-deposited on the first emission layer to form a second emission layer having a thickness of 100 Å. A weight ratio of host:dopant in the second emission layer was 99:1.

T2T was vacuum-deposited on the second emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Examples 22 to 27

Light-emitting devices were manufactured in substantially the same manner as in Comparative Example 9, except that, in forming the emission layer, the type or kind of the mixed host and the weight ratio of the mixed host were changed to those shown in Table 5.

### Example 28

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound EB43 was vacuum-deposited on the hole transport layer to form an electron blocking layer having a thickness of 50 Å.

Compound HD4 as a host and Compound D1 as a dopant were co-vacuum-deposited on the electron blocking layer to form a first emission layer having a thickness of 100 Å. A weight ratio of host:dopant in the first emission layer was 99:1.

Compound HD35 as a host and Compound D1 as a dopant were co-vacuum-deposited on the first emission layer to form a second emission layer having a thickness of 100 Å. A weight ratio of host:dopant in the second emission layer was 99:1.

T2T was vacuum-deposited on the emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Example 29

As an anode, an ITO glass substrate was cut to a size of 50 mm×50 mm×0.5 mm, ultrasonically cleaned with isopropyl alcohol and distilled water each for 10 minutes, and then cleaned by irradiation of ultraviolet rays and exposure to ozone thereto for 10 minutes. Then, the ITO glass substrate was loaded onto a vacuum deposition apparatus.

1,4,5,8,9,11-hexaazatriphenylene-hexacarbonitrile (HAT-CN) was vacuum-deposited on the anode to form a hole injection layer having a thickness of 100 Å. 4,4′-bis[N-(1-naphthyl)-N-phenyl amino]biphenyl (hereinafter, NPB) was vacuum-deposited on the hole injection layer to form a hole transport layer having a thickness of 850 Å. Compound EB43 was vacuum-deposited on the hole transport layer to form an electron blocking layer having a thickness of 50 Å.

Compound HD35 as a host and Compound D1 as a dopant were co-vacuum-deposited on the electron blocking layer to form a first emission layer having a thickness of 50 Å. A weight ratio of host:dopant in the first emission layer was 99:1.

Compound HD4 as a host and Compound D1 as a dopant were co-vacuum-deposited on the first emission layer to form a second emission layer having a thickness of 100 Å. A weight ratio of host:dopant in the second emission layer was 99:1.

Compound HD35 as a host and Compound D1 as a dopant were co-vacuum-deposited on the second emission layer to form a third emission layer having a thickness of 50 Å. A weight ratio of host:dopant in the third emission layer was 99:1.

T2T was vacuum-deposited on the third emission layer to form a hole blocking layer having a thickness of 50 Å. TPBi and LiQ were co-vacuum-deposited at a weight ratio of 1:1 on the hole blocking layer to form an electron transport layer having a thickness of 310 Å. LiF was vacuum-deposited on the electron transport layer to form an electron injection layer having a thickness of 10 Å, and Al was vacuum-deposited on the electron injection layer to form a cathode having a thickness of 800 Å, thereby completing the manufacture of a light-emitting device.

### Evaluation Example 1

To evaluate characteristics of the light-emitting devices manufactured in Comparative Examples 1 to 9 and Examples 1 to 29, driving voltages at 1,000 candela per square meter (cd/m²), luminescence efficiencies (candela per ampere (cd/A)), and lifespans (T₉₀, hr) were measured by utilizing a Keithley MU 236 and a luminance meter PR650,

TABLE 1

| | Electron | | | Driving | Luminescence | Lifespan |
|---|---|---|---|---|---|---|
| | blocking | | | voltage | efficiency | T₉₇ |
| | layer | Host | Dopant | (V) | (cd/A) | (hr) |
| Comparative | CE1 | HH1 | D1 | 3.9 | 7.5 | 75 |
| Example 1 | | | | | | |
| Comparative | CE1 | HH6 | D1 | 3.6 | 6.8 | 98 |
| Example 2 | | | | | | |
| Comparative | CE1 | HD4 | D1 | 3.9 | 7.5 | 102 |
| Example 3 | | | | | | |
| Comparative | CE1 | HD35 | D1 | 3.6 | 6.8 | 125 |
| Example 4 | | | | | | |
| Comparative | CE2 | HD4 | D1 | 3.5 | 7.3 | 108 |
| Example 5 | | | | | | |

TABLE 2

| | Electron | | | Driving | Luminescence | Lifespan |
|---|---|---|---|---|---|---|
| | blocking | | | voltage | efficiency | T₉₇ |
| | layer | Host | Dopant | (V) | (cd/A) | (hr) |
| Comparative | EB1 | HH1:HH6 | D1 | 3.5 | 7.3 | 82 |
| Example 6 | | (5:5) | | | | |
| Example 1 | EB1 | HD1:HD30 | D1 | 3.5 | 7.3 | 88 |
| | | (5:5) | | | | |
| Example 2 | EB1 | HD2:HD31 | D1 | 3.5 | 7.3 | 95 |
| | | (5:5) | | | | |
| Example 3 | EB1 | HD3:HD33 | D1 | 3.5 | 7.3 | 103 |
| | | (5:5) | | | | |
| Example 4 | EB1 | HD4:HD35 | D1 | 3.5 | 7.3 | 120 |
| | | (5:5) | | | | |
| Example 5 | EB1 | HD4:HD35 | D1 | 3.3 | 7.1 | 129 |
| | | (3:7) | | | | |
| Example 6 | EB1 | HD4:HD35 | D1 | 3.6 | 7.4 | 115 |
| | | (7:3) | | | | |
| Example 7 | EB1 | HD4/ | D1 | 3.7 | 7.5 | 116 |
| | | HD35 | | | | |
| | | (first | | | | |
| | | emission | | | | |
| | | layer/ | | | | |
| | | second | | | | |
| | | emission | | | | |
| | | layer) | | | | |

TABLE 3

| | Electron | | | Driving | Luminescence | Lifespan |
|---|---|---|---|---|---|---|
| | blocking | | | voltage | efficiency | T₉₇ |
| | layer | Host | Dopant | (V) | (cd/A) | (hr) |
| Comparative | EB41 | HH1:HH6 | D1 | 3.7 | 7.5 | 80 |
| Example 7 | | (5:5) | | | | |
| Example 8 | EB41 | HD1:HD30 | D1 | 3.7 | 7.5 | 85 |
| | | (5:5) | | | | |
| Example 9 | EB41 | HD2:HD31 | D1 | 3.7 | 7.5 | 90 |
| | | (5:5) | | | | |
| Example 10 | EB41 | HD3:HD33 | D1 | 3.7 | 7.5 | 99 |
| | | (5:5) | | | | |
| Example 11 | EB41 | HD4:HD35 | D1 | 3.7 | 7.5 | 113 |
| | | (5:5) | | | | |
| Example 12 | EB41 | HD4:HD35 | D1 | 3.5 | 7.3 | 120 |
| | | (3:7) | | | | |
| Example 13 | EB41 | HD4:HD35 | D1 | 3.8 | 7.6 | 110 |
| | | (7:3) | | | | |
| Example 14 | EB41 | HD4/ | D1 | 3.9 | 7.7 | 108 |
| | | HD35 | | | | |
| | | (first | | | | |
| | | emission | | | | |
| | | layer/ | | | | |
| | | second | | | | |
| | | emission | | | | |
| | | layer) | | | | |

TABLE 4

| | Electron | | | Driving | Luminescence | Lifespan |
|---|---|---|---|---|---|---|
| | blocking | | | voltage | efficiency | T₉₇ |
| | layer | Host | Dopant | (V) | (cd/A) | (hr) |
| Comparative | EB42 | HH1:HH6 | D1 | 3.5 | 7.2 | 90 |
| Example 8 | | (5:5) | | | | |
| Example 15 | EB42 | HD1:HD30 | D1 | 3.5 | 7.2 | 92 |
| | | (5:5) | | | | |
| Example 16 | EB42 | HD2:HD31 | D1 | 3.5 | 7.2 | 106 |
| | | (5:5) | | | | |
| Example 17 | EB42 | HD3:HD33 | D1 | 3.5 | 7.2 | 111 |
| | | (5:5) | | | | |
| Example 18 | EB42 | HD4:HD35 | D1 | 3.5 | 7.2 | 129 |
| | | (5:5) | | | | |
| Example 19 | EB42 | HD4:HD35 | D1 | 3.4 | 7.0 | 135 |
| | | (3:7) | | | | |
| Example 20 | EB42 | HD4:HD35 | D1 | 3.6 | 7.3 | 122 |
| | | (7:3) | | | | |
| Example 21 | EB42 | HD4/ | D1 | 3.7 | 7.4 | 115 |
| | | HD35 | | | | |
| | | (first | | | | |
| | | emission | | | | |
| | | layer/ | | | | |
| | | second | | | | |
| | | emission | | | | |
| | | layer) | | | | |

TABLE 5

| | Electron | | | Driving | Luminescence | Lifespan |
|---|---|---|---|---|---|---|
| | blocking | | | voltage | efficiency | T₉₇ |
| | layer | Host | Dopant | (V) | (cd/A) | (hr) |
| Comparative | EB43 | HH1:HH6 | D1 | 3.6 | 7.4 | 98 |
| Example 9 | | (5:5) | | | | |
| Example 22 | EB43 | HD1:HD30 | D1 | 3.6 | 7.4 | 102 |
| | | (5:5) | | | | |
| Example 23 | EB43 | HD2:HD31 | D1 | 3.6 | 7.4 | 116 |
| | | (5:5) | | | | |
| Example 24 | EB43 | HD3:HD33 | D1 | 3.6 | 7.4 | 121 |
| | | (5:5) | | | | |
| Example 25 | EB43 | HD4:HD35 | D1 | 3.6 | 7.4 | 138 |
| | | (5:5) | | | | |
| Example 26 | EB43 | HD4:HD35 | D1 | 3.4 | 7.2 | 145 |
| | | (3:7) | | | | |
| Example 27 | EB43 | HD4:HD35 | D1 | 3.7 | 7.5 | 128 |
| | | (7:3) | | | | |
| Example 28 | EB43 | HD4/ | D1 | 3.8 | 7.6 | 120 |
| | | HD35 | | | | |
| | | (first | | | | |
| | | emission | | | | |
| | | layer/ | | | | |
| | | second | | | | |
| | | emission | | | | |
| | | layer) | | | | |
| Example 29 | EB43 | HD35/ | D1 | 3.6 | 7.5 | 133 |
| | | HD4/ | | | | |
| | | HD35 | | | | |
| | | (first | | | | |
| | | emission | | | | |
| | | layer/ | | | | |
| | | second | | | | |
| | | emission | | | | |
| | | layer/third | | | | |
| | | emission | | | | |
| | | layer) | | | | |

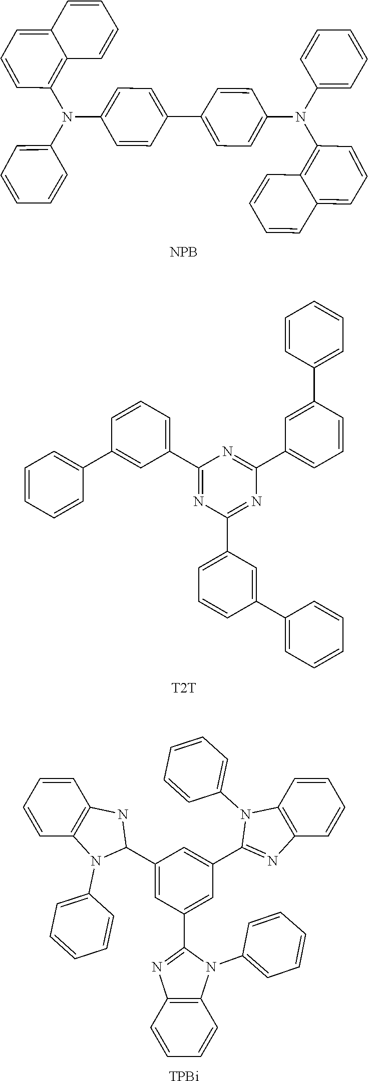

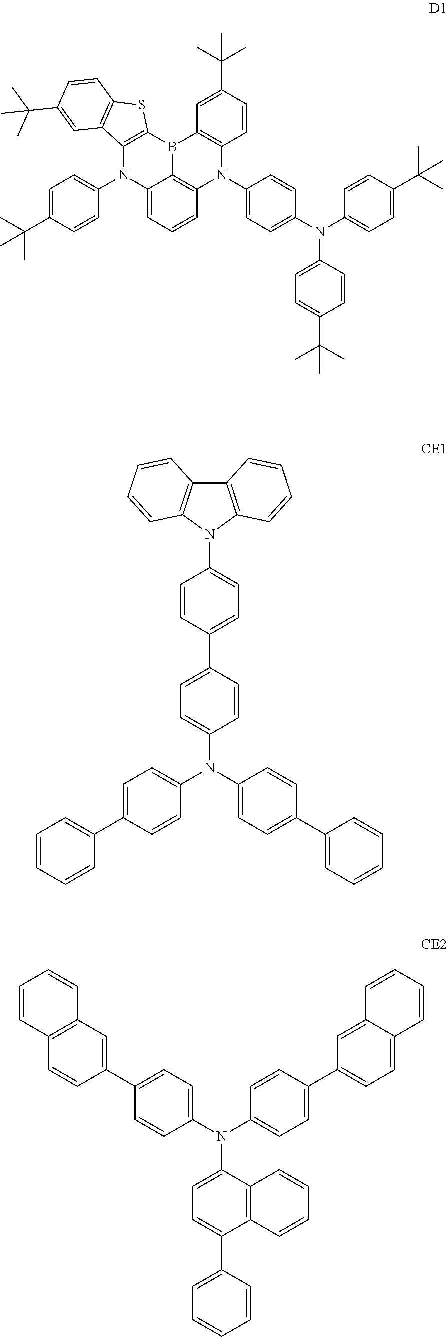 [SMILES: CC(C)(C)c1ccc(N(c2ccc(N3c4ccc(C(C)(C)C)cc4B4c5sc6ccc(C(C)(C)C)cc6c5N(c5ccc(C(C)(C)C)cc5)c5cccc3c54)cc2)c2ccc(C(C)(C)C)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccc5ccccc5c4)cc3)c3ccc(-c4ccc5ccccc5c4)cc3)c3ccccc23)cc1]

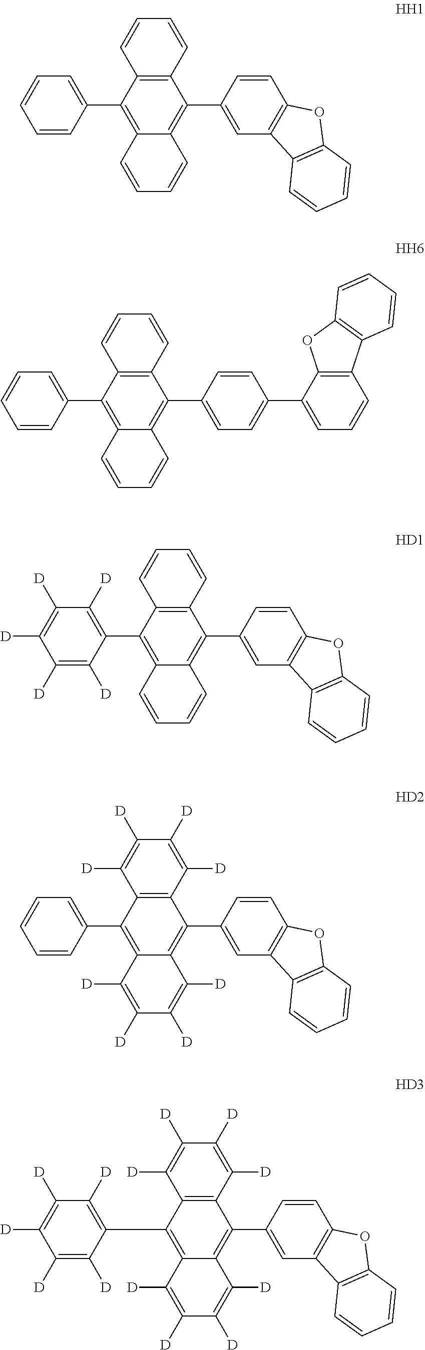 [SMILES: c1ccc(-c2c3ccccc3c(-c3ccc4oc5ccccc5c4c3)c3ccccc23)cc1 ; c1ccc(-c2c3ccccc3c(-c3ccc(-c4cccc5c4oc4ccccc45)cc3)c3ccccc23)cc1 ; [2H]c1c([2H])c([2H])c(-c2c3ccccc3c(-c3ccc4oc5ccccc5c4c3)c3ccccc23)c([2H])c1[2H] ; [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c(-c2c3c([2H])c([2H])c([2H])c([2H])c3c(-c3ccc4oc5ccccc5c4c3)c3c([2H])c([2H])c([2H])c([2H])c23)c([2H])c1[2H]]

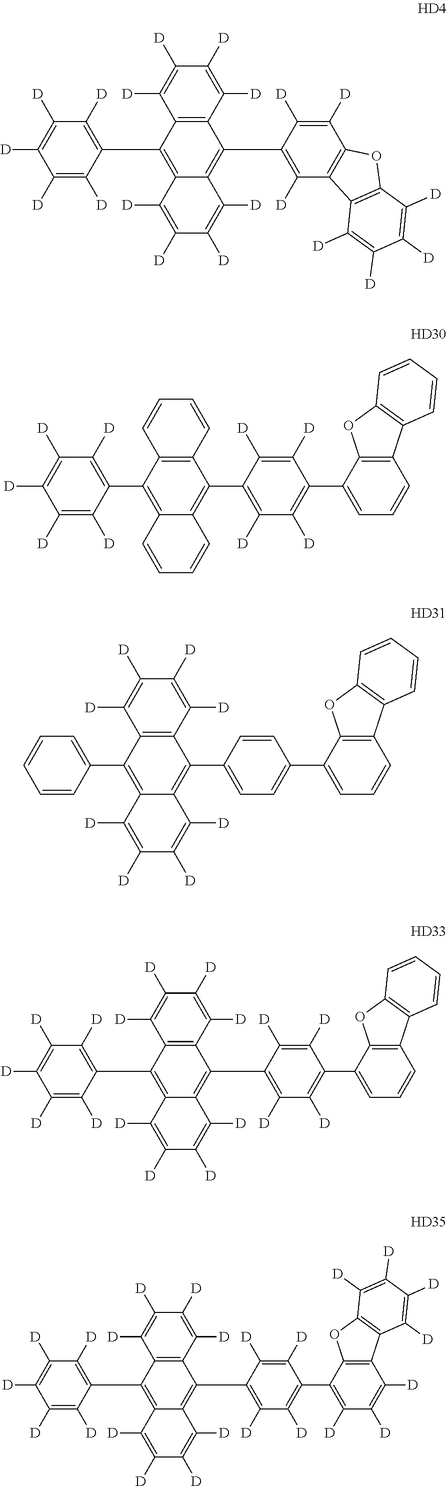

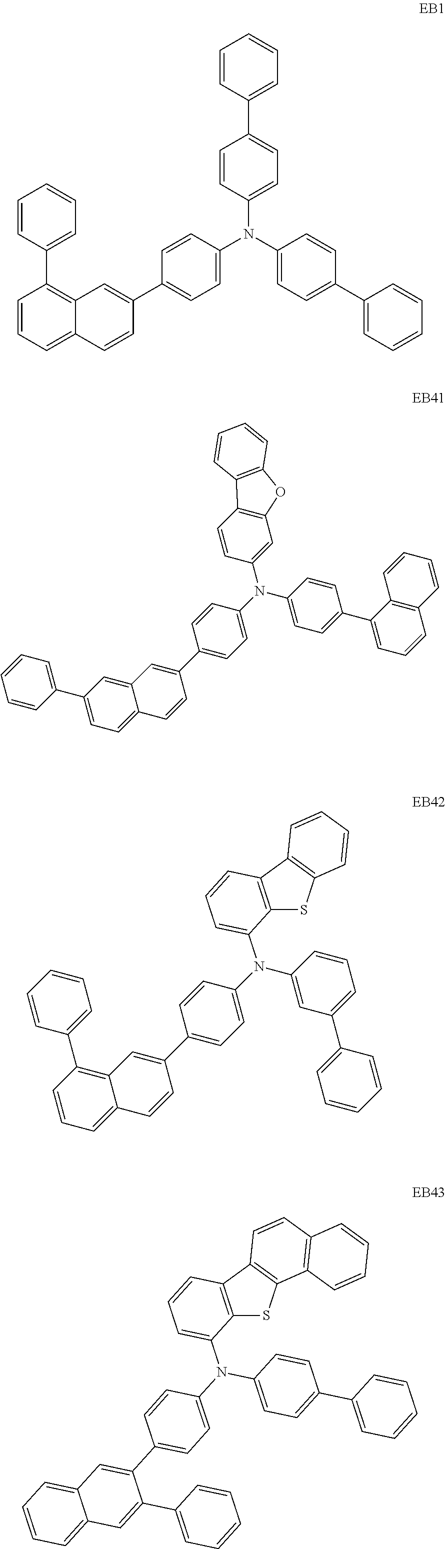 [SMILES: c1ccc(-c2ccc(N(c3ccc(-c4ccccc4)cc3)c3ccc(-c4ccc5cccc(-c6ccccc6)c5c4)cc3)cc2)cc1 ; c1ccc(-c2ccc3ccc(-c4ccc(N(c5ccc(-c6cccc7ccccc67)cc5)c5ccc6c(c5)oc5ccccc56)cc4)cc3c2)cc1 ; c1ccc(-c2cccc(N(c3ccc(-c4ccc5cccc(-c6ccccc6)c5c4)cc3)c3cccc4c3sc3ccccc34)c2)cc1 ; c1ccc(-c2ccc(N(c3ccc(-c4cc5ccccc5cc4-c4ccccc4)cc3)c3cccc4c3sc3c5ccccc5ccc43)cc2)cc1]

From Tables 1 to 5, it is confirmed that because each of the light-emitting devices of Examples 1 to 29 included the first compound and the second compound, which are represented by Formula 1, each include deuterium, and are different from each other, and the third compound represented by Formula 2, the light-emitting devices of Examples 1 to 29 had better driving voltages, better luminescence efficiencies, and/or better lifespans than those of the light-emitting devices of Comparative Examples 1 to 9.

A light-emitting device may have improved driving voltage, improved luminescence efficiency, and/or improved lifespan by including the first compound and the second compound, which are represented by Formula 1, each include deuterium, and are different from each other, and the third compound represented by Formula 2.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the drawings, it will be understood by those of ordinary skill in the art that one or more suitable changes in form and details may be made therein without departing from the spirit and scope as defined by the following claims and equivalents thereof.

What is claimed is:

1. A light-emitting device comprising:
a first electrode;
a second electrode facing the first electrode; and
an interlayer between the first electrode and the second electrode and comprising an emission layer,
wherein the interlayer comprises a first compound, a second compound, and a third compound,
the first compound is represented by Formula 1 and comprises deuterium,
the second compound is represented by Formula 1 and comprises deuterium,
the first compound and the second compound are different from each other, and
the third compound is represented by Formula 2: 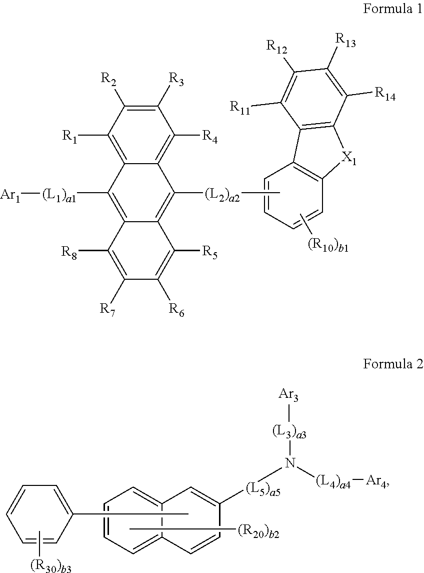
and
wherein, in Formulae 1 and 2,
L₁ to L₅ are each independently a C₃-C₆₀ carbocyclic group that is unsubstituted or substituted with at least one R₁₀ₐ or a C₁-C₆₀ heterocyclic group that is unsubstituted or substituted with at least one R₁₀ₐ,
a1 to a5 are each independently an integer selected from 0 to 2,
Ar₁, Ar₃, and Ar₄ are each independently a C₃-C₆₀ carbocyclic group that is unsubstituted or substituted with at least one R₁₀ₐ or a C₁-C₆₀ heterocyclic group that is unsubstituted or substituted with at least one R₁₀ₐ,
X₁ is C(R₁₅)(R₁₆), N(R₁₅), O, or S,
R₁ to R₈, R₁₀ to R₁₆, R₂₀, and R₃₀ are each independently hydrogen, deuterium, —F, —Cl, —Br, —I, a hydroxyl group, a cyano group, a nitro group, a C₁-C₆₀ alkyl group that is unsubstituted or substituted with at least one R₁₀ₐ, a C₂-C₆₀ alkenyl group that is unsubstituted or substituted with at least one R₁₀ₐ, a C₂-C₆₀ alkynyl group that is unsubstituted or substituted with at least one R₁₀ₐ, a C₁-C₆₀ alkoxy group that is unsubstituted or substituted with at least one R₁₀ₐ, a C₁-C₆₀ alkylthio group that is unsubstituted or substituted with at least one R₁₀ₐ, a C₃-C₅ carbocyclic group that is unsubstituted or substituted with at least one R₁₀ₐ, a C₁-C₆₀ heterocyclic group that is unsubstituted or substituted with at least one R₁₀ₐ, a C₆-C₆₀ aryloxy group that is unsubstituted or substituted with at least one R₁₀ₐ, a C₆-C₆₀ arylthio group that is unsubstituted or substituted with at least one R₁₀ₐ, —Si(Q₁)(Q₂)(Q₃), —N(Q₁)(Q₂), —B(Q₁)(Q₂), —C(═O)(Q₁), —S(═O)₂(Q₁), or —P(═O)(Q₁)(Q₂),
b1 is an integer selected from 0 to 3,
b2 is an integer selected from 0 to 6,
b3 is an integer selected from 0 to 5,
R₁₀ₐ is:
deuterium; —F; —C₁; —Br; —I; a hydroxyl group; a cyano group; or a nitro group;
a C₁-C₆₀ alkyl group, a C₂-C₆₀ alkenyl group, a C₂-C₆₀ alkynyl group, or a C₁-C₆₀ alkoxy group, each unsubstituted or substituted with deuterium, —F, —Cl, —Br, —I, a hydroxyl group, a cyano group, a nitro group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₁₁)(Q₁₂)(Q₁₃), —N(Q₁₁)(Q₁₂), —B(Q₁₁)(Q₁₂), —C(═O)(Q₁₁), —S(═O)₂(Q₁₁), —P(═O)(Q₁₁)(Q₁₂), or a combination thereof;
a C₃-C₆₀ carbocyclic group, a C₁-C₆₀ heterocyclic group, a C₆-C₆₀ aryloxy group, or a C₆-C₆₀ arylthio group, a C₇-C₆₀ arylalkyl group, or a C₂-C₆₀ heteroarylalkyl group, each unsubstituted or substituted with deuterium, —F, —Cl, —Br, —I, a hydroxyl group, a cyano group, a nitro group, a C₁-C₆₀ alkyl group, a C₂-C₆₀ alkenyl group, a C₂-C₆₀ alkynyl group, a C₁-C₆₀ alkoxy group, a C₃-C₆₀ carbocyclic group, a C₁-C₆₀ heterocyclic group, a C₆-C₆₀ aryloxy group, a C₆-C₆₀ arylthio group, a C₇-C₆₀ arylalkyl group, a C₂-C₆₀ heteroarylalkyl group, —Si(Q₂₁)(Q₂₂)(Q₂₃), —N(Q₂₁)(Q₂₂), —B(Q₂₁)(Q₂₂), —C(═O)(Q₂₁), —S(═O)₂(Q₂₁), —P(═O)(Q₂₁)(Q₂₂), or a combination thereof; or
—Si(Q₃₁)(Q₃₂)(Q₃₃); —N(Q₃₁)(Q₃₂); —B(Q₃₁)(Q₃₂); —C(═O)(Q₃₁); —S(═O)₂(Q₃₁); or —P(═O)(Q₃₁)(Q₃₂), and
Q₁ to Q₃, Q₁₁ to Q₁₃, Q₂₁ to Q₂₃, and Q₃₁ to Q₃₃ are each independently:
hydrogen; deuterium; —F; —C₁; —Br; —I; a hydroxyl group; a cyano group; a nitro group; a C₁-C₆₀ alkyl group; a C₂-C₆₀ alkenyl group; a C₂-C₆₀ alkynyl group; or a C₁-C₆₀ alkoxy group; or
a C₃-C₆₀ carbocyclic group or a C₁-C₆₀ heterocyclic group, a C₇-C₆₀ arylalkyl group, or a C₂-C₆₀ heteroarylalkyl group, each unsubstituted or substituted with deuterium, —F, a cyano group, a C₁-C₆₀ alkyl group, a C₁-C₆₀ alkoxy group, a phenyl group, a biphenyl group, or a combination thereof.

2. The light-emitting device of claim 1, wherein the emission layer comprises the first compound and the second compound.

3. The light-emitting device of claim 1, wherein the interlayer further comprises a hole transport region between the first electrode and the emission layer, and
the hole transport region comprises the third compound.

4. The light-emitting device of claim 3, wherein the hole transport region comprises:
an electron blocking layer; and
at least one selected from among a hole injection layer, a hole transport layer, an emission auxiliary layer, and combinations thereof, and the electron blocking layer comprises the third compound.

5. The light-emitting device of claim 4, wherein the electron blocking layer is on the emission layer.

6. The light-emitting device of claim 1, wherein the emission layer comprises a first emission layer and a second emission layer between the first emission layer and the second electrode,
the first emission layer comprises the first compound, and
the second emission layer comprises the second compound.

7. The light-emitting device of claim 1, wherein the interlayer further comprises a fourth compound containing boron.

8. The light-emitting device of claim 1, wherein the emission layer is configured to emit blue light.

9. The light-emitting device of claim 1, wherein the first compound and the second compound are each independently represented by at least one of Formulae 1-1 to 1-4: 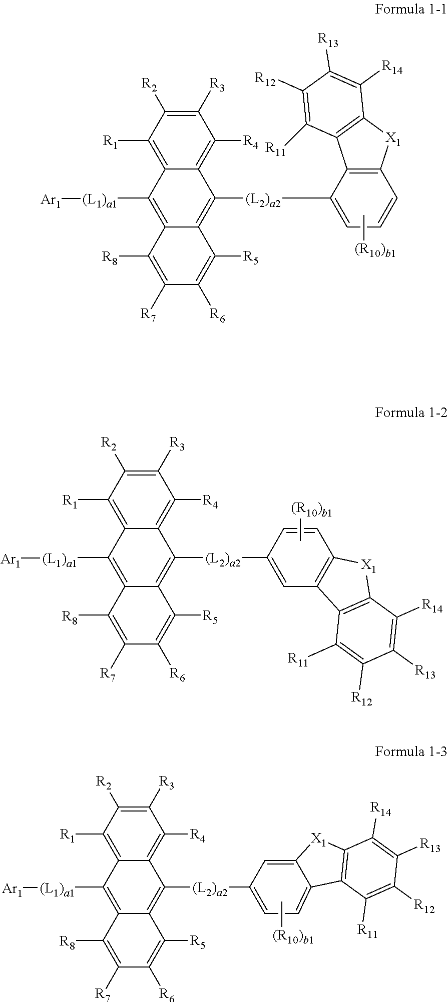 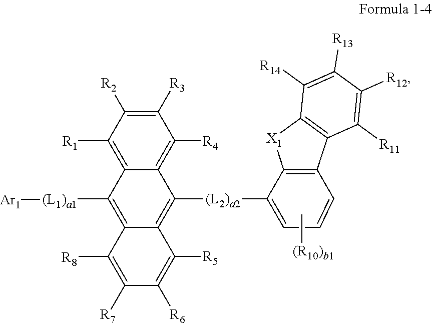
wherein, in Formulae 1-1 to 1-4, L₁, L₂, a1, a2, Ar₁, X₁, R₁ to R₈, R₁₀ to R₁₄, and b1 are each as described in Formula 1.

10. The light-emitting device of claim 1, wherein, Ar₁ is a C₆-C₆₀ aryl group that is unsubstituted or substituted with at least one R₁₀ₐ.

11. The light-emitting device of claim 1, wherein, L₁ and L₂ are each a C₆-C₆₀ arylene group that is unsubstituted or substituted with at least one R₁₀ₐ.

12. The light-emitting device of claim 1, wherein, L₁ and L₂ are each independently a benzene group, a naphthalene group, an anthracene group, a phenanthrene group, a perylene group, a phenalene group, a pyrene group, or a chrysene group, each unsubstituted or substituted with at least one R₁₀ₐ.

13. The light-emitting device of claim 1, wherein, L₁ and L₂ are each independently represented by at least one of Formulae LK1 to LK13: 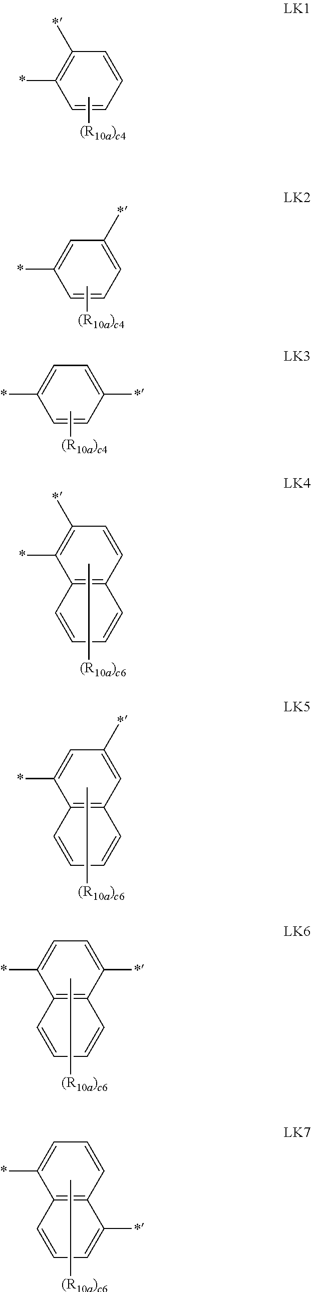 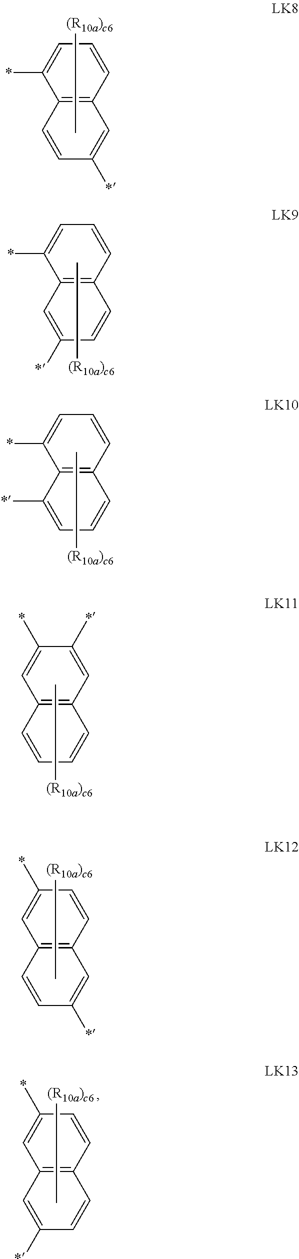
wherein, in Formulae LK1 to LK13,
c4 is an integer selected from 0 to 4,
c6 is an integer selected from 0 to 6, and
* and *′ each indicate a binding site to a neighboring atom.

14. The light-emitting device of claim 1, wherein at least one of the first compound or the second compound satisfies at least one of Conditions 1 to 6:
Condition 1
Ar is a C₃-C₆₀ carbocyclic group that is substituted with at least one deuterium or a C₁-C₆₀ heterocyclic group that is unsubstituted or substituted with at least one deuterium;
Condition 2
L₁ is a C₃-C₆₀ carbocyclic group that is substituted with at least one deuterium or a C₁-C₆₀ heterocyclic group that is unsubstituted or substituted with at least one deuterium;
Condition 3
at least one of R₁ to R₈ is deuterium;
Condition 4
L₂ is a C₃-C₆₀ carbocyclic group that is substituted with at least one deuterium or a C₁-C₆₀ heterocyclic group that is unsubstituted or substituted with at least one deuterium;
Condition 5
at least one R₁₀ is deuterium; and/or
Condition 6
at least one of R₁₁ to R₁₄ is deuterium.

15. The light-emitting device of claim 1, wherein the first compound and the second compound are each independently at least one compound selected from Compounds HH1 to HH11 in which at least one hydrogen is substituted with deuterium:

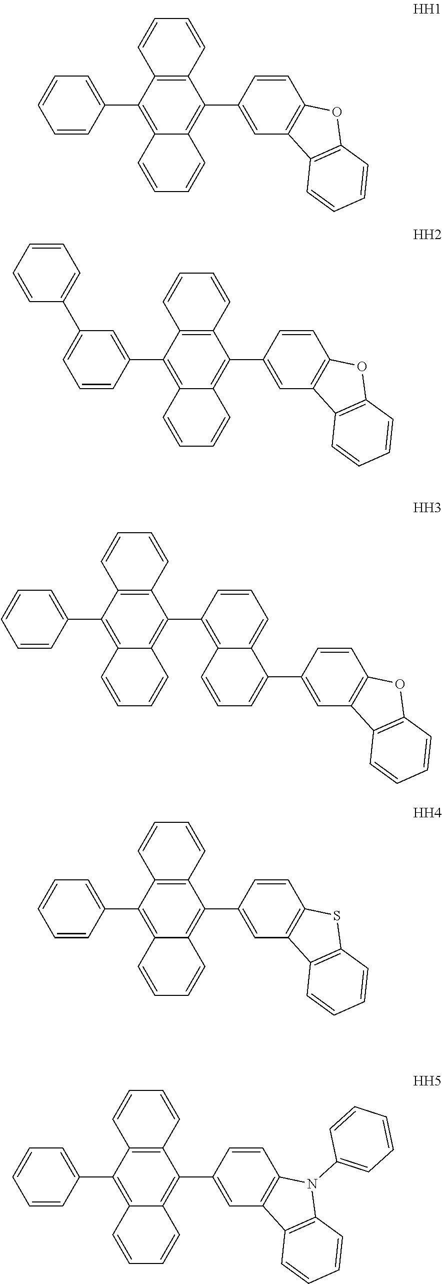 [SMILES: c1ccc(-c2c3ccccc3c(-c3ccc4oc5ccccc5c4c3)c3ccccc23)cc1 ; c1ccc(-c2cccc(-c3c4ccccc4c(-c4ccc5oc6ccccc6c5c4)c4ccccc34)c2)cc1 ; c1ccc(-c2c3ccccc3c(-c3cccc4c(-c5ccc6oc7ccccc7c6c5)cccc34)c3ccccc23)cc1 ; c1ccc(-c2c3ccccc3c(-c3ccc4sc5ccccc5c4c3)c3ccccc23)cc1 ; c1ccc(-c2c3ccccc3c(-c3ccc4c(c3)c3ccccc3n4-c3ccccc3)c3ccccc23)cc1]

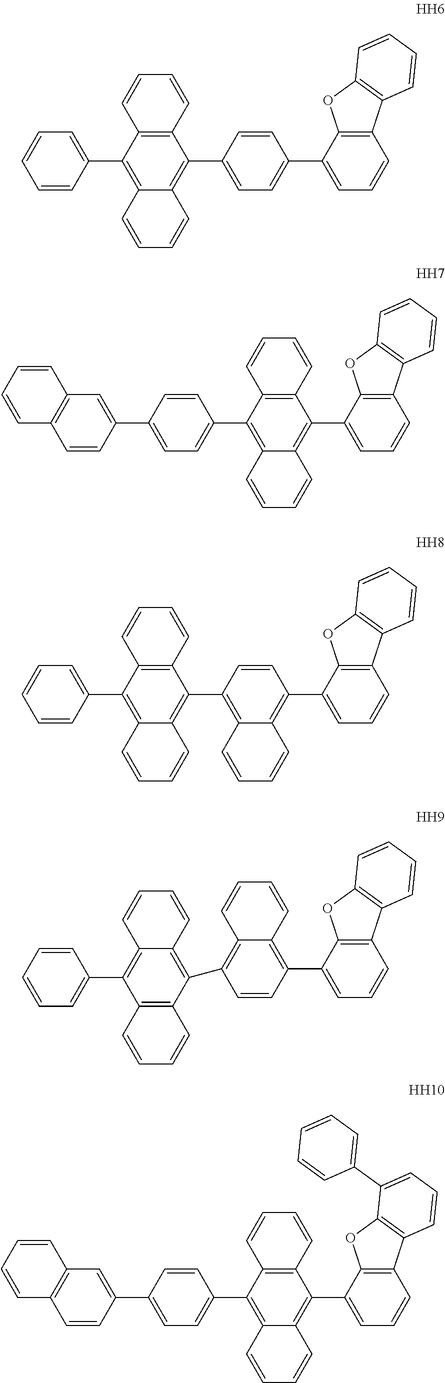 [SMILES: c1ccc(-c2c3ccccc3c(-c3ccc(-c4cccc5c4oc4ccccc45)cc3)c3ccccc23)cc1 ; c1ccc2cc(-c3ccc(-c4c5ccccc5c(-c5cccc6c5oc5ccccc56)c5ccccc45)cc3)ccc2c1 ; c1ccc(-c2c3ccccc3c(-c3ccc(-c4cccc5c4oc4ccccc45)c4ccccc34)c3ccccc23)cc1 ; c1ccc(-c2c3ccccc3c(-c3ccc(-c4cccc5c4oc4ccccc45)c4ccccc34)c3ccccc23)cc1 ; c1ccc(-c2cccc3c2oc2c(-c4c5ccccc5c(-c5ccc(-c6ccc7ccccc7c6)cc5)c5ccccc45)cccc23)cc1]

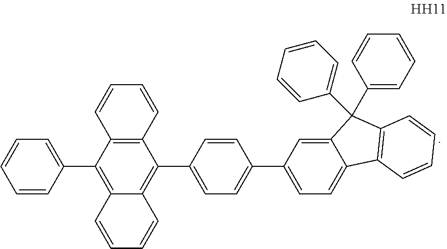 [SMILES: c1ccc(-c2c3ccccc3c(-c3ccc(-c4ccc5c(c4)C(c4ccccc4)(c4ccccc4)c4ccccc4-5)cc3)c3ccccc23)cc1]

16. The light-emitting device of claim 1, wherein, each of R₂₀ and R₃₀ excludes —N(Q₁)(Q₂).

17. The light-emitting device of claim 1, wherein the third compound is represented by at least one of Formulae 2-1 to 2-7: 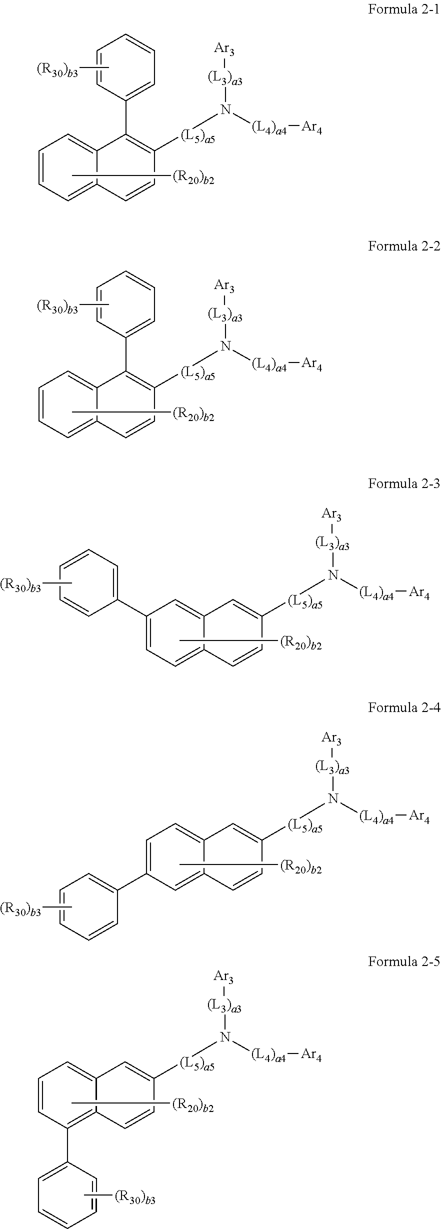 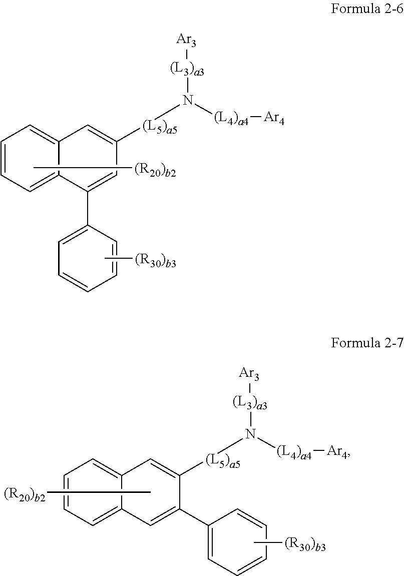
wherein, in Formulae 2-1 to 2-7, L₃ to L₅, a3 to a5, Ar₃, Ar₄, R₂₀, R₃₀, b2, and b3 are each as described in Formula 2.

18. The light-emitting device of claim 1, wherein the third compound is at least one of Compounds EB1 to EB43:

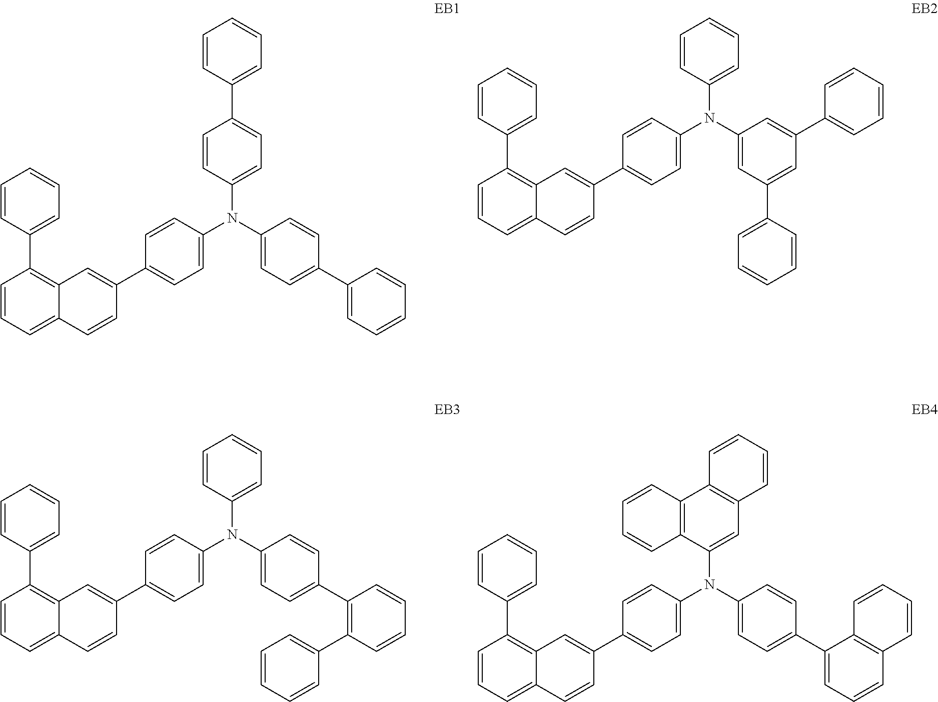 [SMILES: c1ccc(-c2ccc(N(c3ccc(-c4ccccc4)cc3)c3ccc(-c4ccc5cccc(-c6ccccc6)c5c4)cc3)cc2)cc1 ; c1ccc(-c2cc(-c3ccccc3)cc(N(c3ccccc3)c3ccc(-c4ccc5cccc(-c6ccccc6)c5c4)cc3)c2)cc1 ; c1ccc(-c2ccccc2-c2ccc(N(c3ccccc3)c3ccc(-c4ccc5cccc(-c6ccccc6)c5c4)cc3)cc2)cc1 ; c1ccc(-c2cccc3ccc(-c4ccc(N(c5ccc(-c6cccc7ccccc67)cc5)c5cc6ccccc6c6ccccc56)cc4)cc23)cc1]

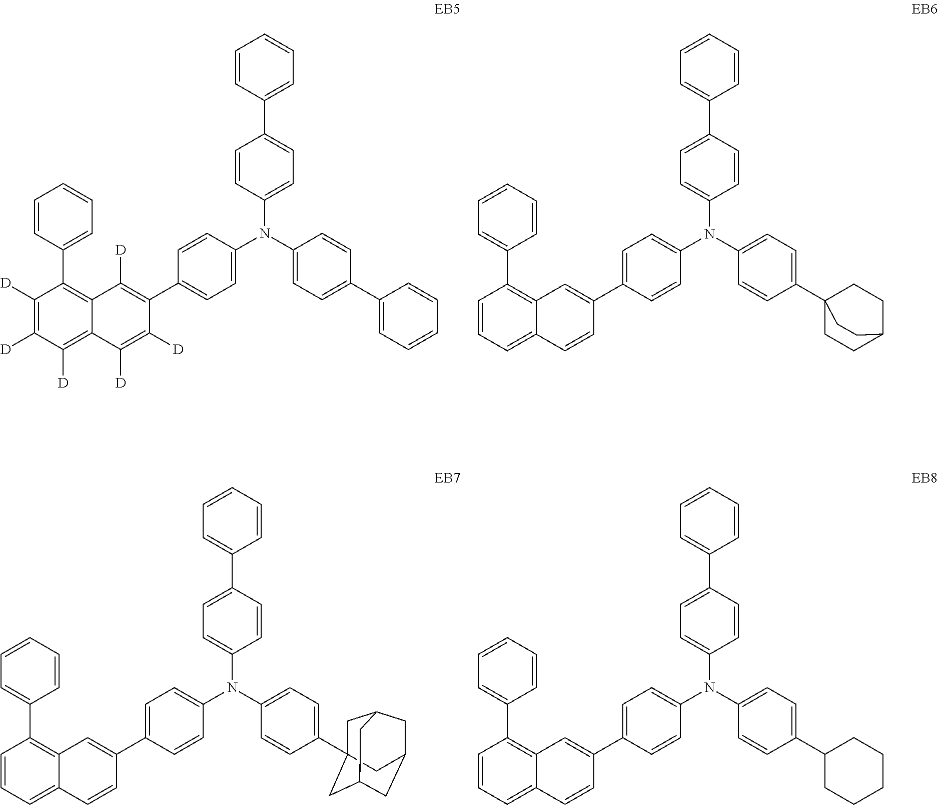

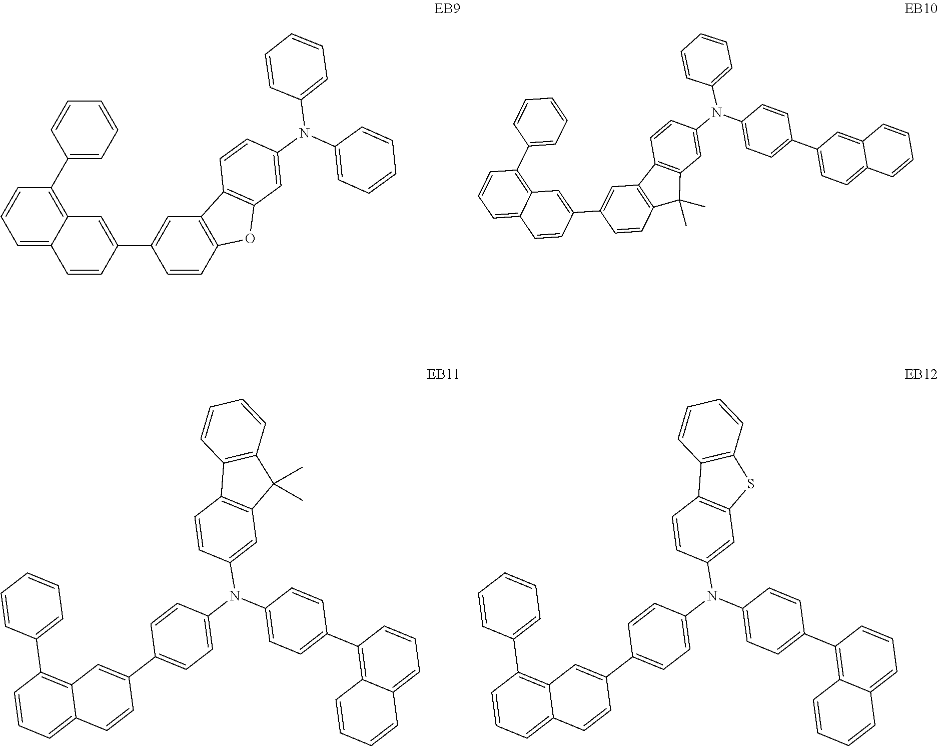 [SMILES: c1ccc(-c2cccc3ccc(-c4ccc5oc6cc(N(c7ccccc7)c7ccccc7)ccc6c5c4)cc23)cc1 ; CC1(C)c2ccc(-c3ccc4cccc(-c5ccccc5)c4c3)cc2-c2ccc(N(c3ccccc3)c3ccc(-c4ccc5ccccc5c4)cc3)cc21 ; CC1(C)c2ccccc2-c2ccc(N(c3ccc(-c4ccc5cccc(-c6ccccc6)c5c4)cc3)c3ccc(-c4cccc5ccccc45)cc3)cc21 ; c1ccc(-c2cccc3ccc(-c4ccc(N(c5ccc(-c6cccc7ccccc67)cc5)c5ccc6c(c5)sc5ccccc56)cc4)cc23)cc1]

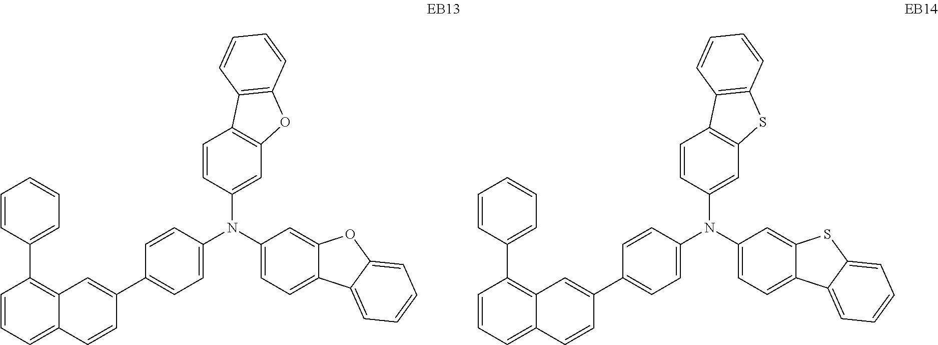 [SMILES: c1ccc(-c2cccc3ccc(-c4ccc(N(c5ccc6c(c5)oc5ccccc56)c5ccc6c(c5)oc5ccccc56)cc4)cc23)cc1 ; c1ccc(-c2cccc3ccc(-c4ccc(N(c5ccc6c(c5)sc5ccccc56)c5ccc6c(c5)sc5ccccc56)cc4)cc23)cc1]

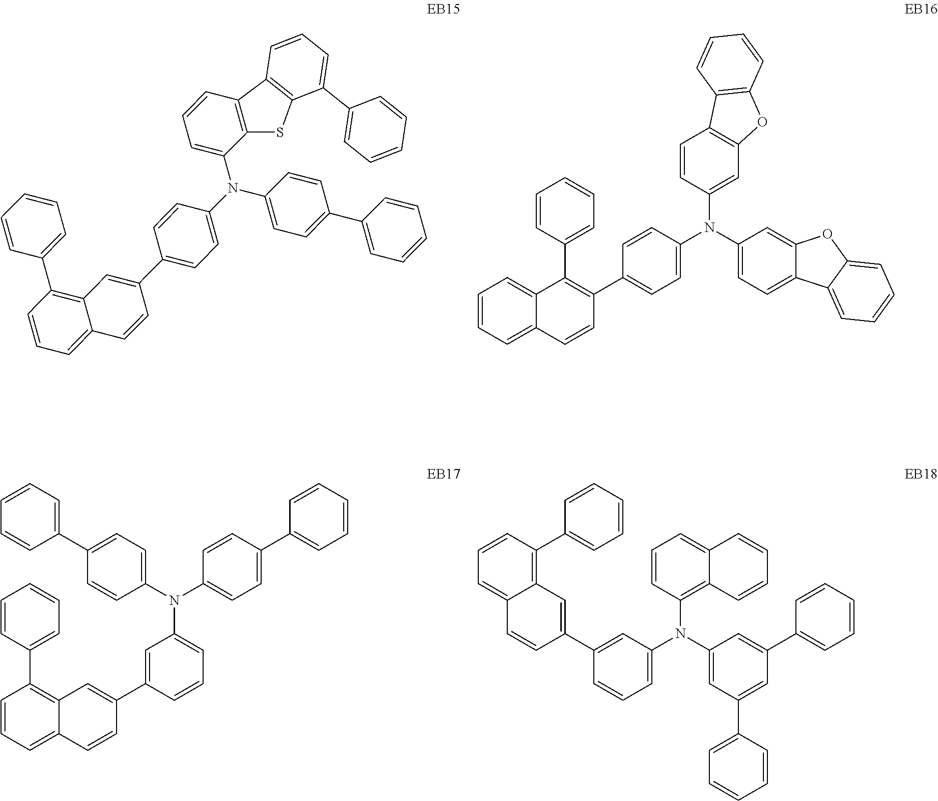 [SMILES: c1ccc(-c2ccc(N(c3ccc(-c4ccc5cccc(-c6ccccc6)c5c4)cc3)c3cccc4c3sc3c(-c5ccccc5)cccc34)cc2)cc1 ; c1ccc(-c2c(-c3ccc(N(c4ccc5c(c4)oc4ccccc45)c4ccc5c(c4)oc4ccccc45)cc3)ccc3ccccc23)cc1 ; c1ccc(-c2ccc(N(c3ccc(-c4ccccc4)cc3)c3cccc(-c4ccc5cccc(-c6ccccc6)c5c4)c3)cc2)cc1 ; c1ccc(-c2cc(-c3ccccc3)cc(N(c3cccc(-c4ccc5cccc(-c6ccccc6)c5c4)c3)c3cccc4ccccc34)c2)cc1]

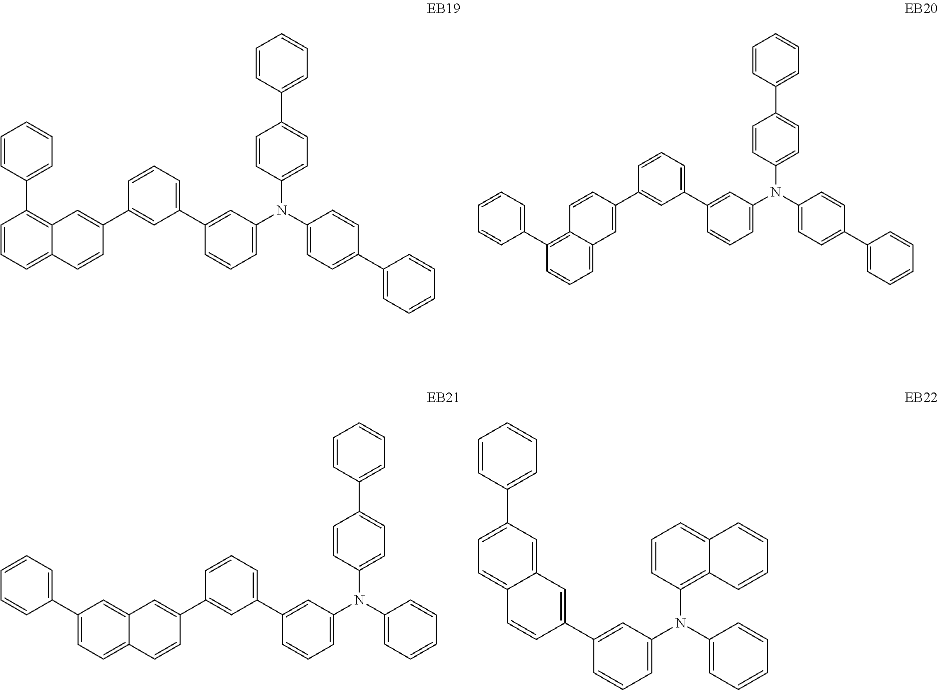 [SMILES: c1ccc(-c2ccc(N(c3ccc(-c4ccccc4)cc3)c3cccc(-c4cccc(-c5ccc6cccc(-c7ccccc7)c6c5)c4)c3)cc2)cc1 ; c1ccc(-c2ccc(N(c3ccc(-c4ccccc4)cc3)c3cccc(-c4cccc(-c5ccc6c(-c7ccccc7)cccc6c5)c4)c3)cc2)cc1 ; c1ccc(-c2ccc(N(c3ccccc3)c3cccc(-c4cccc(-c5ccc6ccc(-c7ccccc7)cc6c5)c4)c3)cc2)cc1 ; c1ccc(-c2ccc3ccc(-c4cccc(N(c5ccccc5)c5cccc6ccccc56)c4)cc3c2)cc1]

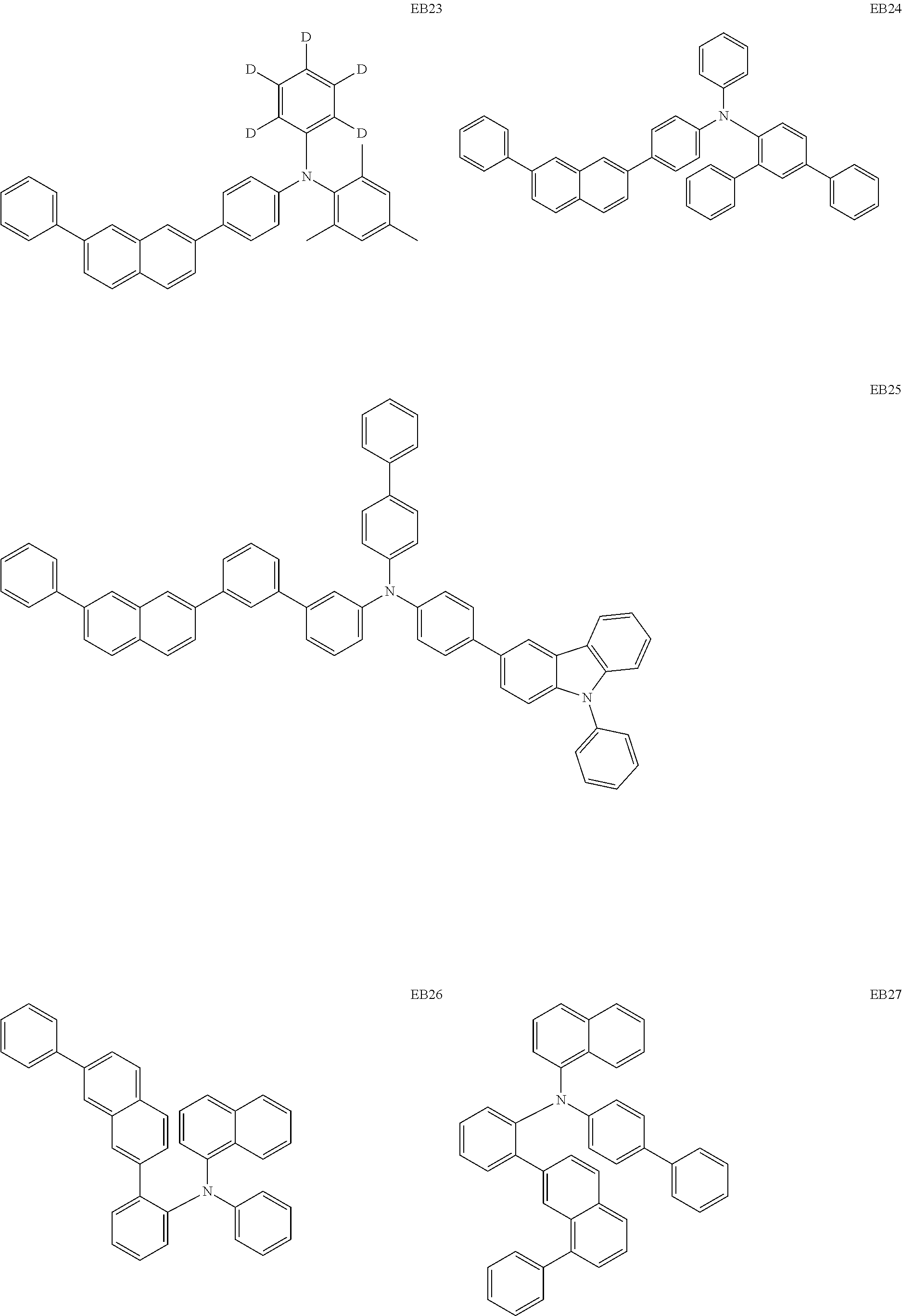 [SMILES: [2H]c1c([2H])c([2H])c(N(c2ccc(-c3ccc4ccc(-c5ccccc5)cc4c3)cc2)c2c(C)cc(C)cc2C)c([2H])c1[2H] ; c1ccc(-c2ccc(N(c3ccccc3)c3ccc(-c4ccc5ccc(-c6ccccc6)cc5c4)cc3)c(-c3ccccc3)c2)cc1 ; c1ccc(-c2ccc(N(c3ccc(-c4ccc5c(c4)c4ccccc4n5-c4ccccc4)cc3)c3cccc(-c4cccc(-c5ccc6ccc(-c7ccccc7)cc6c5)c4)c3)cc2)cc1 ; c1ccc(-c2ccc3ccc(-c4ccccc4N(c4ccccc4)c4cccc5ccccc45)cc3c2)cc1 ; c1ccc(-c2ccc(N(c3ccccc3-c3ccc4cccc(-c5ccccc5)c4c3)c3cccc4ccccc34)cc2)cc1]

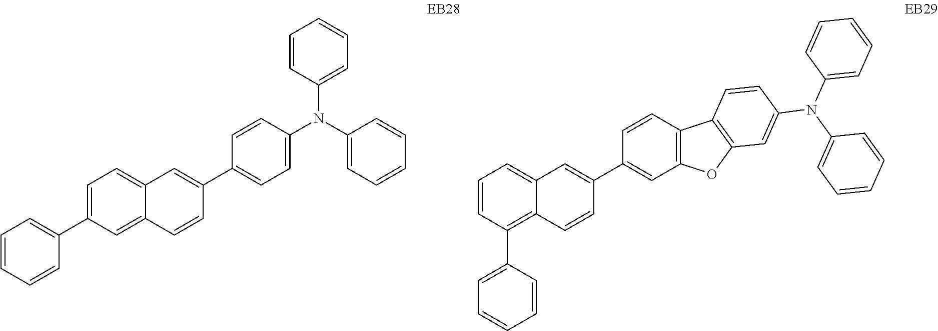 [SMILES: c1ccc(-c2ccc3cc(-c4ccc(N(c5ccccc5)c5ccccc5)cc4)ccc3c2)cc1 ; c1ccc(-c2cccc3cc(-c4ccc5c(c4)oc4cc(N(c6ccccc6)c6ccccc6)ccc45)ccc23)cc1]

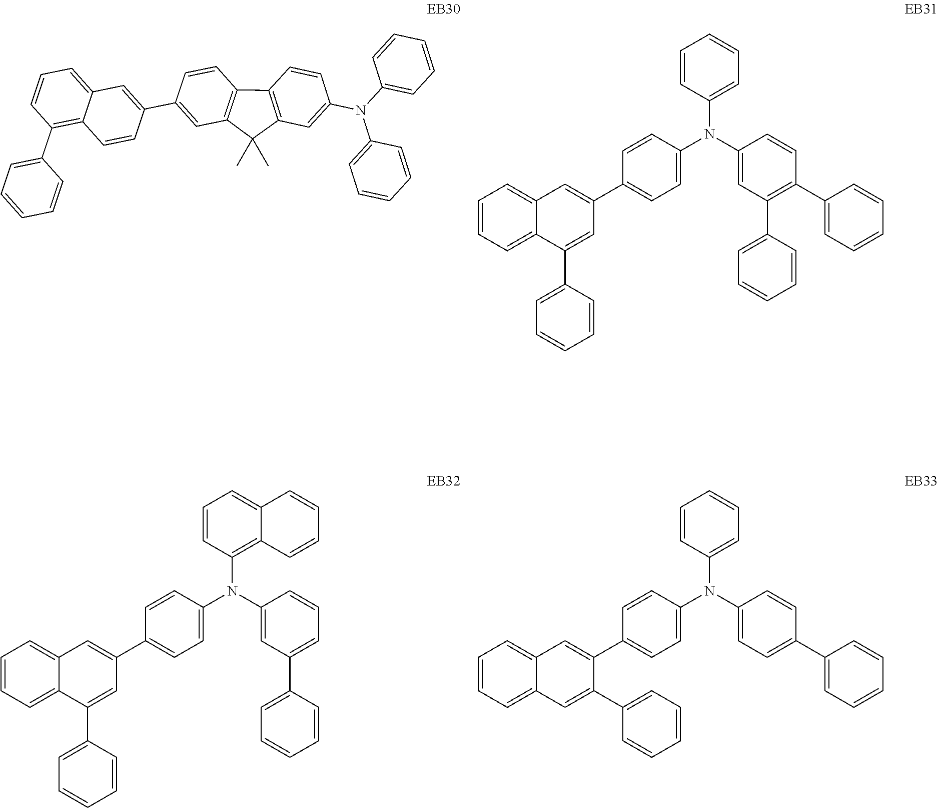 [SMILES: CC1(C)c2cc(-c3ccc4c(-c5ccccc5)cccc4c3)ccc2-c2ccc(N(c3ccccc3)c3ccccc3)cc21 ; c1ccc(-c2ccc(N(c3ccccc3)c3ccc(-c4cc(-c5ccccc5)c5ccccc5c4)cc3)cc2-c2ccccc2)cc1 ; c1ccc(-c2cccc(N(c3ccc(-c4cc(-c5ccccc5)c5ccccc5c4)cc3)c3cccc4ccccc34)c2)cc1 ; c1ccc(-c2ccc(N(c3ccccc3)c3ccc(-c4cc5ccccc5cc4-c4ccccc4)cc3)cc2)cc1]

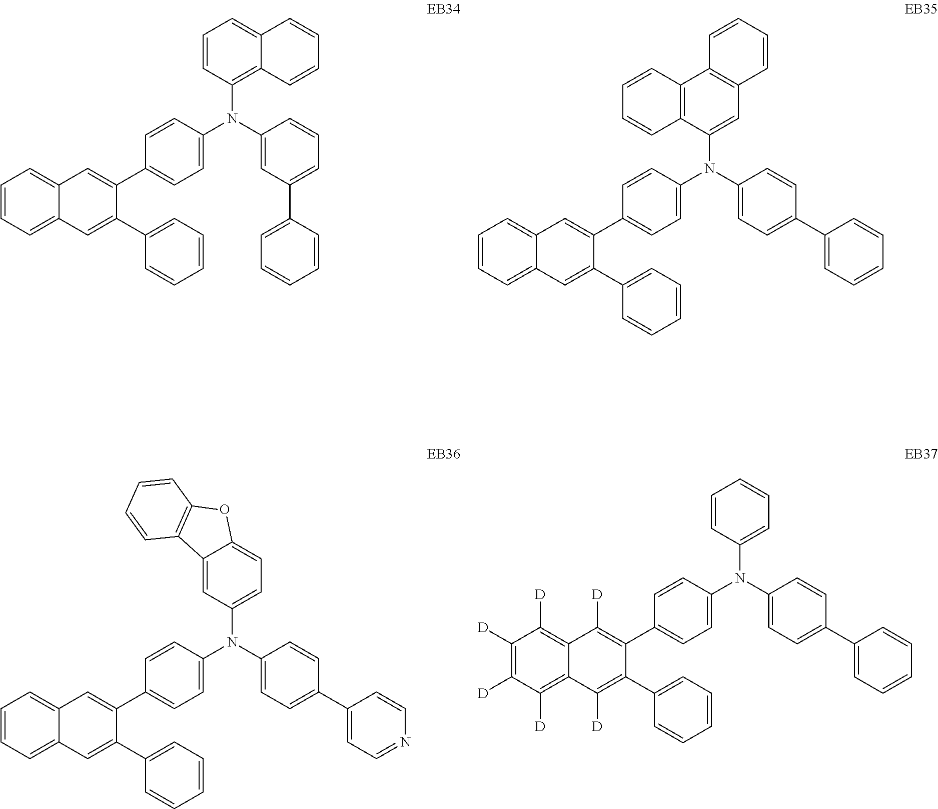 [SMILES: c1ccc(-c2cccc(N(c3ccc(-c4cc5ccccc5cc4-c4ccccc4)cc3)c3cccc4ccccc34)c2)cc1 ; c1ccc(-c2ccc(N(c3ccc(-c4cc5ccccc5cc4-c4ccccc4)cc3)c3cc4ccccc4c4ccccc34)cc2)cc1 ; c1ccc(-c2cc3ccccc3cc2-c2ccc(N(c3ccc(-c4ccncc4)cc3)c3ccc4oc5ccccc5c4c3)cc2)cc1 ; [2H]c1c([2H])c([2H])c2c([2H])c(-c3ccc(N(c4ccccc4)c4ccc(-c5ccccc5)cc4)cc3)c(-c3ccccc3)c([2H])c2c1[2H]]

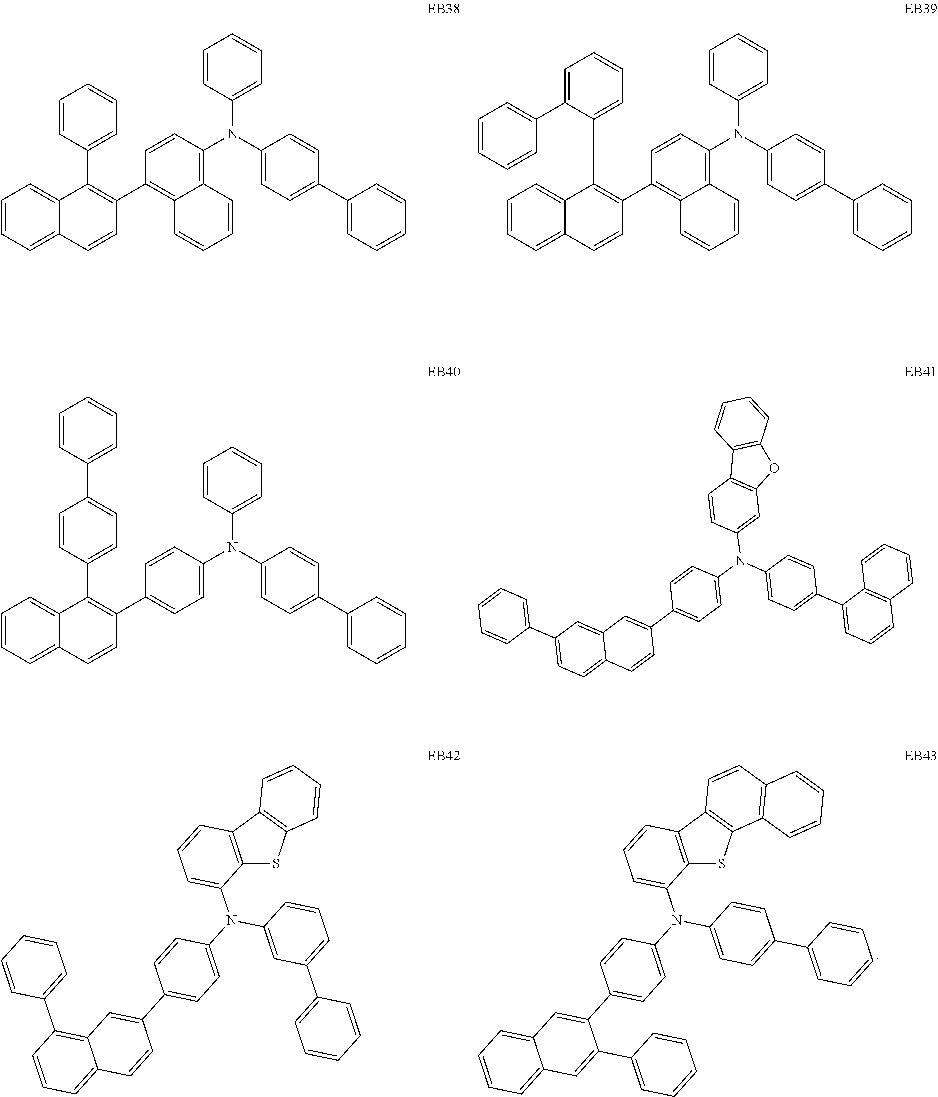 [SMILES: c1ccc(-c2ccc(N(c3ccccc3)c3ccc(-c4ccc5ccccc5c4-c4ccccc4)c4ccccc34)cc2)cc1 ; c1ccc(-c2ccc(N(c3ccccc3)c3ccc(-c4ccc5ccccc5c4-c4ccccc4-c4ccccc4)c4ccccc34)cc2)cc1 ; c1ccc(-c2ccc(-c3c(-c4ccc(N(c5ccccc5)c5ccc(-c6ccccc6)cc5)cc4)ccc4ccccc34)cc2)cc1 ; c1ccc(-c2ccc3ccc(-c4ccc(N(c5ccc(-c6cccc7ccccc67)cc5)c5ccc6c(c5)oc5ccccc56)cc4)cc3c2)cc1 ; c1ccc(-c2cccc(N(c3ccc(-c4ccc5cccc(-c6ccccc6)c5c4)cc3)c3cccc4c3sc3ccccc34)c2)cc1 ; c1ccc(-c2ccc(N(c3ccc(-c4cc5ccccc5cc4-c4ccccc4)cc3)c3cccc4c3sc3c5ccccc5ccc43)cc2)cc1]

19. An electronic apparatus comprising:
the light-emitting device of claim 1; and
a thin-film transistor electrically connected to the light-emitting device.

20. An electronic equipment comprising the light-emitting device of claim 1, wherein the electronic equipment is at least one selected from among a flat panel display, a curved display, a computer monitor, a medical monitor, a television, a billboard, an indoor or outdoor light and/or light for signal, a head-up display, a fully or partially transparent display, a flexible display, a rollable display, a foldable display, a stretchable display, a laser printer, a telephone, a portable phone, a tablet personal computer, a phablet, a personal digital assistant (PDA), a wearable device, a laptop computer, a digital camera, a camcorder, a viewfinder, a micro display, a three-dimensional (3D) display, a virtual reality or augmented reality display, a vehicle, a video wall with multiple displays tiled together, a theater or stadium screen, a phototherapy device, a signboard, and combinations thereof.
